(19) United States
(12) Patent Application Publication
(10) Pub. No.: US 2023/0086868 A1
(43) Pub. Date: Mar. 23, 2023

(54) METHODS AND COMPOSITIONS FOR TREATING RETINAL DISEASES AND CONDITIONS

(71) Applicants: Lineage Cell Therapeutics, Inc., Carlsbad, CA (US); Hadasit Medical Research Services and Development Ltd., Jerusalem (IL)

(72) Inventors: Francois Binette, Napa, CA (US); Gary Hogge, San Francisco, CA (US); Rami Skaliter, Ness Ziona (IL); Avi Ben Shabat, Tel-Aviv (IL); Jordi Monés, Barcelona (ES); Eyal Banin, Jerusalem (IL); Tina Guanting Qiu, Carlsbad, CA (US)

(21) Appl. No.: 18/058,425

(22) Filed: Nov. 23, 2022

Related U.S. Application Data
(63) Continuation of application No. PCT/US2021/034114, filed on May 25, 2021.
(60) Provisional application No. 63/029,669, filed on May 25, 2020.
(60) Provisional application No. 63/036,327, filed on Jun. 8, 2020.
(60) Provisional application No. 63/106,339, filed on Oct. 27, 2020.
(60) Provisional application No. 63/182,684, filed on Apr. 30, 2021.

(51) Int. Cl. A61K 35/30; A61K 9/00; A61P 27/02; A61P 9/10

(52) U.S. Cl. CPC A61K 35/30; A61K 9/0048; A61P 27/02; A61P 9/10

(57) ABSTRACT

Provided herein are methods, compositions of matter, and devices for treating diseases and illnesses of the eye, including retinal conditions such as macular degeneration.

### CROSS-REFERENCES TO RELATED APPLICATIONS

This application is a continuation of PCT International Application No. PCT/US2021/034114 with an international filing date of May 25, 2021, which claims the benefit of U.S. Provisional Application No. 63/029,669, filed May 25, 2020; U.S. Provisional Application No. 63/036,327, filed Jun. 8, 2020; U.S. Provisional Application No. 63/106,339, filed Oct. 27, 2020; and U.S. Provisional Application No. 63/182,684, filed Apr. 30, 2021, which are incorporated herein by reference in their entireties and for all purposes.

### BACKGROUND

The present disclosure pertains generally to the field of treating retinal diseases, and more particularly to treating retinal diseases using human embryonic stem cell derived retinal pigment epithelial (RPE) cell compositions.

Dysfunction, degeneration and loss of RPE cells are prominent features of retinal diseases such as AMD. Best Disease and subtypes of Retinitis Pigmentosa (RP). AMD is the leading cause of visual disability in the Western world. Among people over 75 years of age. 25-30% are affected by Age-Related Macular Degeneration (AMD), with progressive central visual loss that leads to blindness in 6-8% of the patients. AMD involves multiple etiological risk factors such as aging, smoke and complement polymorphism, its pathophysiological root causes can be summarized as RPE aging, oxidative stress, para inflammation, Bruch's membrane aging and choroidal ischemia, which individually or collectively trigger the metabolic deterioration of the retinal health. The retinal degeneration primarily involves the macula, the central part of the retina responsible for fine visual detail, color perception, facial recognition, reading, and driving. There are two forms of AMD: wet AMD and dry AMD. Dry AMD is the more common of the two types, accounting for approximately 85-90% of cases. Wet AMD is the less common of the two types, accounting for approximately 10-15% of cases. The dry form of AMD is initiated by hyperplasia of the RPE and formation of drusen deposits underneath the RPE or within the Bruch's membrane consisting of metabolic end products. The disease may gradually progress into the advanced stage of geographic atrophy (GA) with degeneration of RPE cells and photoreceptors over large areas of the macula, causing central visual loss. In addition, degeneration RPE affects the blood-retinal barrier (BRB), which is composed of an inner and an outer barrier. The outer BRB refers to the barrier formed at the retinal pigment epithelial cell layer along with the Bruch's membrane, which regulates the solutes and nutrients from the choroid to the subretinal space. The outer BRB plays essential role in maintaining the anatomic and functional integrities of photoreceptors, especially within the macular region where the highest oxygen metabolic activities in the body are undertaken. The primary goal of hRPE cell therapy is to replace the loss or damaged host RPE and deliver functional, active and viable RPE to support the photoreceptor.

The pathogenesis of the disease involves abnormalities in four functionally interrelated tissues. i.e., retinal pigment epithelium (RPE). Bruch's membrane, Choriocapillaris, and photoreceptors. However, impairment of RPE cell function is an early and crucial event in the molecular pathways leading to clinically relevant AMD changes.

Dry age-related macular degeneration (AMD) is a leading cause of adult blindness in the developed world. Nearly all cases of wet AMD begin as dry AMD. Dry AMD typically affects both eyes. There are currently no U.S. Food and Drug Administration (FDA) or European Medicines Agency (EMA) approved treatment options available for patients with dry AMD. Prophylactic measures include vitamin/mineral supplements. These reduce the risk of developing wet AMD but do not affect the development of progression of geographic atrophy.

### SUMMARY

Embodiments herein generally relate to methods, compositions of matter, and devices for treating diseases and illnesses of the eye, including retinal conditions such as macular degeneration.

In an aspect, the present disclosure provides a method of treating or slowing the progression of a retinal disease or disorder, comprising administering a cell therapeutic agent to a subject in need thereof, wherein the cell therapeutic agent comprises retinal pigment epithelium (RPE) cells, and wherein the RPE cells restore the anatomy or functionality of a retina of the subject.

In some embodiments, the RPE cells are derived from pluripotent cells. In some embodiments, the RPE cells are human RPE cells. In some embodiments, the RPE cells are derived from a human embryonic (hESC) cell line.

In some embodiments, the RPE cells were derived under low oxygen (5%) culture supplemented with high concentration of Activin A, a transforming growth factor beta (TGF-b) family member, and nicotinamide before switching to normal oxygen (20%) culture to enrich the RPE population.

In some embodiments, the RPE cells secrete PEDF at a concentration of about 2000 ng/ml/day to about 4000 ng/ml/day.

In some embodiments, the cell therapeutic agent is administered to a region of the atrophic retina or adjacent to a region of the atrophic retina of the patient.

In some embodiments, the cell therapeutic agent is administered at a dose of about 50,000 cells to about 1,000,000 cells. In some embodiments, the cell therapeutic agent is administered at a dose of about 100,000 cells to about 750.000 cells. In some embodiments, the cell therapeutic agent is administered at a dose of about 200,000 cells to about 500,000 cells.

In some embodiments, the administration of the cell therapeutic agent decreases the atrophy area in an atrophic retina of the subject.

In some embodiments, the administration of the cell therapeutic agent restores one or more retinal layers of the retina.

In some embodiments, the administration of the cell therapeutic agent restores the functionality of photoreceptors in the retina.

In some embodiments, the administration of the cell therapeutic agent restores the outer nuclear layer (ONL) of the retina.

In some embodiments, the administration of the cell therapeutic agent restores the ellipsoid zone (EZ) of the retina.

In some embodiments, the administration of the cell therapeutic agent restores the fovea of the retina.

In some embodiments, the administration of the cell therapeutic agent restores the blood-retinal barrier (BRB) of the retina.

In some embodiments, the administration of the cell therapeutic agent remodels the extracellular matrix (ECM) of the retina.

In some embodiments, the restoring of the anatomy or functionality of the retina is determined by assessing one or more of reduced growth of geographic atrophy, improvement of visual acuity, improvement of reading speed, improvement of retinal structure, reductions in drusen, or stable engraftment of cells.

In some embodiments, the improvement is measured by microperimetry.

In some embodiments, the vision of the subject is improved by treatment, and the improved vision is assessed by one or more of: change in total area of GA lesion(s); change in monocular reading speed; change in Functional Reading Independence Index (FRII) composite score; change in normal luminance best-corrected visual acuity score (NL-BCVA); change in low luminance best corrected visual acuity score (LL-BCVA); change in low luminance deficit (LLD); change in monocular critical print size; change in the National Eye Institute Visual Functioning Questionnaire 25 Item Version (NEI VFQ-25) distance activity subscale score; change in number of scotomatous points; change in macular sensitivity; and change in systemic plasma concentration of APL-2.

In some embodiments, the method results in minimal or no delayed inflammation of rejection of implanted cells.

In some embodiments, administering comprises delivering the RPE cells to a region of the retina or adjacent to the retina. In some embodiments, delivering comprises implanting the RPE cells in a region of the retina or adjacent to the retina.

In some embodiments, the treating comprises the pluripotent secretory effects of the RPE cells.

In some embodiments, the subject suffers from a retinal disease condition selected from Dry AMD, retinitis pigmentosae, usher syndrome, vitelliform maculopathy, Stargardt disease, retinal detachment, retinal dysplasia, retinal atrophy, retinopathy, macular dystrophy, cone dystrophy, cone-rod dystrophy. Malattia Leventinese, Doyne honeycomb dystrophy, Sorsby's dystrophy, pattern/butterfly dystrophies, Best vitelliform dystrophy. North Carolina dystrophy, central areolar choroidal dystrophy, angioid streaks, toxic maculopathy, pathologic myopia, retinitis pigmentosa, and macular degeneration.

In some embodiments, the cell therapeutic agent is administered with a delivery device.

In some embodiments, the cell therapeutic agent is administered to or adjacent to a geographic atrophy of the retina with the delivery device.

In some embodiments, the delivery device comprises a needle, a capillary and a tip. In some embodiments, the delivery device comprises a needle with an outer diameter of about 0.63 mm and an inner diameter of about 0.53 mm, a capillary with an outer diameter of about 0.5 mm and an inner diameter of about 0.25 mm, and a tip with an outer diameter of about 0.12 mm and an inner diameter of about 0.07 mm.

In another aspect, the present disclosure provides a delivery device for use with any of the methods described herein.

In some embodiments, the delivery device comprises a needle, a capillary and a tip.

In some embodiments, the device comprises a needle with an outer diameter of about 0.63 mm and an inner diameter of about 0.53 mm, a capillary with an outer diameter of about 0.5 mm and an inner diameter of about 0.25 mm, and a tip with an outer diameter of about 0.12 mm and an inner diameter of about 0.07 mm.

In yet another aspect, the present disclosure provides a composition comprising a cell therapeutic agent for restoring the anatomy or functionality of a retina in a subject according to the present disclosure.

The retinal pigment epithelium (RPE) is a monolayer of neuroepithelium-derived pigmented cells that lays on a Bruch's membrane between the photoreceptor outer segments (POS) and the choroidal vasculature. The RPE monolayer is critical to the function and health of the photoreceptors. Dysfunction, injury, and loss of retinal pigment epithelium (RPE) cells are prominent features of certain eye diseases and disorders, such as age-related macular degeneration (AMD), hereditary macular degenerations including Best disease (the early onset form of vitelliform macular dystrophy), and subtypes of retinitis pigmentosa (RP). The transplantation of RPE into the retina of those affected with such diseases can be used as cell replacement therapy in retinal diseases where RPE have degenerated.

Human pluripotent stem cells provide significant advantages as a source of RPE cells for transplantation. Their pluripotent developmental potential enables their differentiation into authentic functional RPE cells, and given their potential for infinite self-renewal, they can serve as an unlimited source of RPE cells. Indeed, it has been demonstrated that human embryonic stem cells (hESCs) and human induced pluripotent stem cells (iPSCs) may differentiate into RPE cells in vitro, attenuate retinal degeneration and preserve visual function after subretinal implantation. Therefore, hESCs can be an unlimited source for the production of RPE cells for cell therapy.

However, most cell-based treatments are usually preserved frozen in a cryo-solution that is not compatible with direct administration into the body, creating a practical problem for clinical use. Cells should be transplanted within hours after they are thawed, or they may begin to lose viability and quality. In addition, cells must be prepared prior to administration in certified facilities, which may not be in close proximity to clinical sites, hospitals or other treatment facilities. Finally, each subject's treatment dose must be released by a qualified technician since preparation of the final formulation is considered to be part of the cell therapy production process.

The present disclosure addresses these and other shortcomings in the field of regenerative medicine and RPE cell therapy. The disclosure further provides data related to various methods, devices and compositions of matter.

Teachings, methods, compositions of matter, devices and know-how for the instant embodiments are found in PCT Publication Nos. WO 2019/130061, published Jul. 4, 2019 entitled “RETINAL PIGMENT EPITHELIUM CELL COMPOSITIONS;” WO 2018/170494, published Sep. 20, 2018, entitled “METHODS FOR MEASURING THERAPEUTIC EFFECTS OF RETINAL DISEASE THERAPIES;” and WO 2017/017686, published Feb. 2, 2017, entitled “LARGE SCALE PRODUCTION OF RETINAL PIGMENT EPITHELIAL CELLS;” each of which is incorporated herein by reference in its entirety for all of its methods, devices and apparatuses, compositions of matter, alone or in combination with each other.

### BRIEF DESCRIPTION OF THE DRAWINGS

FIG. 1 shows a retinal scan showing a pigmented area (arrows) within the Geographic Atrophy (GA) of subject 18 at 3 months post-treatment with RPE cells, evidencing the presence of RPE cells in the inferior area of the GA. The area of RPE cell transplantation represented by a white circle is also known as the bleb area, which is a blister-like formation resulting from injection of the RPE cells.

FIG. 2 shows a retinal scan showing a pigmented area (arrows) within the GA of subject 18 at 9 months post-treatment, evidencing the presence of RPE cells in the inferior area of the GA.

FIG. 3 is a graph showing change in visual acuity based on change in number of Early Treatment Diabetic Retinopathy Study (ETDRS) letters from baseline for each of 12 subjects after the indicated treatment. Almost all the subjects maintain their baseline BCVA, and more than half had a steady improvement in BCVA.

FIG. 4 is a graph showing mean change in size of GA (mm²) from baseline over time, for the treated and untreated (fellow) eyes in Cohort 4. The data demonstrate that GA growth was slower on the treated eye compared to the fellow eye.

FIG. 5 is a graph showing change in visual acuity based on mean change in number of ETDRS letters from baseline over time, for the treated and untreated (fellow) eyes in Cohort 4. The data demonstrate that BCVA reduction was less severe on the treated eye compared to the fellow eye.

FIG. 6 is a graph showing mean change in number of ETDRS letters from baseline over time, for the treated and untreated (fellow) eyes in subject 22. Subject demonstrated substantial improvement and gain of visual functional activity on treated eye vs reduction on fellow eye.

FIGS. 7A-7C show the changes over time for subject 14. FIG. 7A is a graph showing mean change in number of ETDRS letters from baseline over time, for the treated and untreated (fellow) eyes. FIG. 7B is a graph showing mean change in size of GA (mm²) from baseline over time, for the treated and untreated (fellow) eyes. FIG. 7C shows the number of letters read at baseline and 3 years post-treatment in the treated and untreated (fellow) eyes. Subject demonstrated substantial difference between treated and fellow eye on both anatomical and visual functional aspects in favor of the treated eye.

FIG. 8 is a graph showing change in reading speed (words per minute) from baseline over time in the treated (left panel) and untreated (fellow, right panel) eyes of individual subjects from Cohort 4. Data demonstrate functional clinical visual improvement on treated vs the fellow eye.

FIG. 9 shows high resolution optical coherence tomography (OCT) images from the treated retina of subject 14 at baseline (top) and 9 months post-treatment (bottom). Left images indicate region of retina shown in right images. The boundaries of GA demonstrate outer retinal layer restoration/regeneration at 9 months.

FIG. 10 shows OCT images of the treated retina of subject 14 before starting the study (historical, orange, left panel), at baseline (red), 9 months (blue), and 23 months (yellow) post-treatment. Regression of GA from baseline was observed at both 9 and 23 months after treatment, demonstrating anatomical improvement and outer retinal regeneration/restoration.

FIG. 11 is a graph showing change of total size of GA (total area in square root transformation, SQRT) in both eyes of subject 14 and rate of change in mm SQRT/yr from previous and from baseline (expected growth from historical plotted). Yellow hatched bars indicate predicted/expected growth for the fellow (FE), non-treated eye. Blue hatched bars indicate predicted/expected growth for the study treated eye.

FIG. 12 is OCT retinal images from the treated eye of subject 14, showing GA boundaries based on ELM border at baseline (top) and 3 months after treatment (bottom). ELM borders are shown by red arrows and dotted lines. Change in ELM border from baseline (BSL) to 3 months (3M) is indicated by large arrows. Outer plexiform layer is shown by blue arrows. New RPE cells are shown by small green arrows in bottom image. Left images indicate region of retina shown in right images. Central growth of the ELM border and/or ONL/OPL, as well as new presumable RPE, are already observed at 3M post-treatment.

FIG. 13 is OCT retinal images from the treated eye of subject 14, showing GA boundaries based on ELM border at baseline (top) and 5 months after treatment (bottom). ELM borders are shown by red arrows and dotted lines. Change in ELM border from baseline (BSL) to 5 months (5M) is indicated by large arrows. Outer plexiform layer is shown by blue arrows. New RPE cells are shown by small green arrows. Left images indicate region of retina shown in right images. Central growth of the ELM border and/or ONL/OPL, as well as new presumable RPE, are observed at 5M post-treatment.

FIG. 14 is OCT retinal images from the treated eye of subject 14, showing GA boundaries based on ELM border at baseline (top), 9 months (center), and 23 months (bottom) after treatment. ELM borders are shown by red arrows and dotted lines. Change in ELM border from baseline (BSL) to 9 months (9M) is indicated by large arrows. Change from 9M to 23 months (23M) is indicated by medium arrows. Outer plexiform layer is shown by blue arrows. New RPE cells are shown by small green arrows. Left images indicate region of retina shown in right images. Central growth of the ELM border and/or ONL/OPL, as well as new presumable RPE, are observed at 9M post-treatment, with a small regression at 23M.

FIG. 15 shows changes in a microperimetry test of the treated eye of subject 14 at 23 months (23M) and 35 months (35M) post-treatment. FIG. 15 demonstrates an improvement of the visual function and reduction of the scotoma (“blind spot/area” represented as a black stain in the orange circle), and improvement in light sensitivity on 35M compared to 23M. Microperimetry is a fundus related visual filed test that captures the specific area of vision in the macula area and generates a high resolution and accurate mapping of retinal sensitivity areas. Microperimetry is a better test to assess changes in visual function with higher reliability than a “simple” BCVA test. Moreover, microperimetry supplies accurate correlation between anatomical changes and defect to visual function defect.

FIG. 16 is OCT retinal images from the treated eye of subject 21, showing GA boundaries based on ELM border at baseline (top) and 1 month after treatment (bottom). ELM borders are shown by arrows and dotted lines. OPL borders are shown by arrows. Change in ELM border from baseline (BSL) to 1 month (1M) is indicated by arrow between the dotted lines. Left images indicate region of retina shown in right images. Central growth of the ELM border and/or OPL are observed at 1M post-treatment.

FIG. 17 is an infrared (IR) image of the retina of subject 21. GA boundaries at baseline and 1 month are indicated.

FIG. 18 is OCT retinal images from the treated eye of subject 21, showing an isolated atrophic lesion at baseline (top) and 3 months after treatment (bottom). Left images indicate region of retina shown in right images. New features (circled) suggest outer retinal regeneration at 3 months. Almost complete restoration of the previously atrophic area with regeneration of the missing layers and “disappearance” of the atrophic lesion was observed.

FIG. 19 is OCT retinal images from the treated eye of subject 21, showing GA at baseline (top) and 3 months after treatment (bottom). Left images indicate region of retina shown in right images. A new hyper-reflective monolayer likely shows RPE cells, and possible restoration of ELM, OPL and ONL at 3 months.

FIG. 20 is OCT retinal images from the treated eye of subject 21, showing GA at baseline (top) and 3 months after treatment (bottom). Left images indicate region of retina shown in right images. A very thin but homogenous and continuous layer of ONL (circled), with preserved ELM and a RPE monolayer over an area of choroidal hypertransmission, is not normally present but was observed at 3 months post-treatment. This indicates restored new layers within an atrophic region.

FIG. 21 shows images of an isolated atrophic lesion in the retina of subject 21 before (baseline, top left), 1 month (middle left) and 2 months (bottom left) after administration of OpRegen-RPE. Right image indicates region of retina shown in left images.

FIG. 22 images of the superior GA region in the retina of subject 21 before (baseline, top left), 1 month (middle left) and 2 months (bottom left) after administration of OpRegen-RPE. Right image indicates region of retina shown in left images.

FIG. 23 shows changes over time for subject 22. Left panel is a graph showing mean change in number of ETDRS letters from baseline over time, for the treated and untreated (fellow) eyes. Right panel is a graph showing mean change in size of GA (mm²) from baseline over time, for the treated and untreated (fellow) eyes. Data demonstrate substantial difference between treated and fellow eye on both anatomical and visual functional aspects in favor of the treated eye. Substantial visual acuity improvement was observed on the treated eye.

FIG. 24 is a fundus photography (FP) image showing fine pigmentary motting at 3 months post-treatment (right panel), but not baseline (left panel) in the retina of subject 22, indicating presence of RPE cells at 3 months.

FIG. 25 is an IR image of the retina at baseline (left) and 3 months post-treatment (right) for subject 22. GA borders are reduced and less defined at 3 months.

FIG. 26 is OCT retinal images from the treated eye of subject 22, showing central GA at baseline (top) and 3 months after treatment (bottom). Left images indicate region of retina shown in right images. Baseline boundary of atrophy is shown by line. New features, including less subsidence of outer plexiform, new ELM within the area of atrophy, new RPE within the area of atrophy, and less hypertransmission are indicated with small arrows.

FIG. 27 is OCT retinal images from the treated eye of subject 22, showing inferior GA at baseline (top) and 3 months after treatment (bottom). Left images indicate region of retina shown in right images. Baseline boundary of atrophy is shown by line. New features, including less subsidence of outer plexiform, new ELM within the area of atrophy, and new RPE within the area of atrophy are indicated with small arrows.

FIG. 28 is OCT retinal images from the treated eye of subject 22, showing an isolated atrophic lesion at baseline (top) and 3 months after treatment (bottom). Left images indicate region of retina shown in right images. Baseline boundary of atrophy is shown by line. New features, including less subsidence of outer plexiform, new ELM within the area of atrophy, and new RPE within the area of atrophy are indicated with small arrows.

FIG. 29 is OCT retinal images showing the boundaries of the GA at baseline (left) and 3 months (right), based on the ELM border. Total area, growth rate, and SQRT transformation growth rate are indicated.

FIG. 30 is OCT retinal images showing the central GA area for subject 22 at baseline (top left), 2 months (middle left) and 3 months (bottom left) post-treatment. New subretinal material (RPE cells) was observed at 2 months, with increased subretinal material and reformation of ELM observed at 3 months (arrows). Right images indicate region of retina shown in left images. The blue circles are progressive coordinates showing blood vessels of the choroid used to mark the same location and capture the exact area of the retina on subsequent visits.

FIG. 31 is retinal images showing the area of RPE delivery in subject 14 at baseline (top left), during surgery (Intra OP, top right), 2 months (bottom left) and 3 months (bottom right) past-treatment. The bleb represents the area of cell delivery. Bleb covered the GA during surgery, indicating full coverage of the GA with RPE cells.

FIG. 32 is retinal images showing intraoperative imaging of the blebs representing the area of RPE cell delivery for subjects 19 (left) and 21 (right). GA indicated by arrows.

FIGS. 33A-33C are spectral domain optical coherence tomography (SD-OCT) images. FIG. 33A shows an example B-scan. FIG. 33B is the B-scan from FIG. 33A, with boundaries between the layers overlaid. FIG. 33C is the B-scan from FIG. 33A, with layer thickness overlaid.

FIG. 34 shows an example illustration of thickness and area maps generated from SD-OCT. Tissue loss is indicated by the white area, preserved tissue area is indicated by gray or black. Relative thickness of the total retina (left panel), outer nuclear layer (second from left), photoreceptors outer segments (second from right), and RPE+drusen complex (right panel) are shown.

FIG. 35 shows total retina thickness maps for the treated eye (top) and untreated eye (bottom) from subject 8 at baseline (left), 3 months (second from left), 6 months (second from right) and 12 months (right) post-treatment. Average total thickness is indicated.

FIG. 36 shows thickness maps of the outer nuclear layer (ONL) for the treated eye (top) and untreated eye (bottom) from subject 8 at baseline (left), 3 months (second from left), 6 months (second from right) and 12 months (right) post-treatment. Total area of the ONL is indicated.

FIG. 37 shows thickness maps of the photoreceptors outer segments for the treated eye (top) and untreated eye (bottom) from subject 8 at baseline (left), 3 months (second from left), 6 months (second from right) and 12 months (right) post-treatment. Total area of the photoreceptors outer segments is indicated.

FIG. 38 shows thickness maps of the RPE and drusen complex for the treated eye (top) and untreated eye (bottom) from subject 8 at baseline (left), 3 months (second from left), 6 months (second from right) and 12 months (right) post-treatment. Total area of the RPE and drusen complex is indicated.

FIG. 39 shows total retina thickness maps for the treated eye (top) and untreated eye (bottom) from subject 5 at baseline (left), 6 months (center) and 12 months (right) post-treatment. Average total thickness is indicated.

FIG. 40 shows thickness maps of the outer nuclear layer (ONL) for the treated eye (top) and untreated eye (bottom) from subject 5 at baseline (left), 6 months (center) and 12 months (right) post-treatment. Total area of the ONL is indicated.

FIG. 41 shows thickness maps of the photoreceptors outer segments for the treated eye (top) and untreated eye (bottom) from subject 5 at baseline (left), 6 months (center) and 12 months (right) post-treatment. Total area of the photoreceptors outer segments is indicated.

FIG. 42 shows thickness maps of the RPE and drusen complex for the treated eye (top) and untreated eye (bottom) from subject 5 at baseline (left), 6 months (center) and 12 months (right) post-treatment. Total area of the RPE and drusen complex is indicated.

FIG. 43 shows total retina thickness maps for the treated eye (top) and untreated eye (bottom) from subject 13 at baseline (left), 6 months (center) and 12 months (right) post-treatment. Average total thickness is indicated.

FIG. 44 shows thickness maps of the outer nuclear layer (ONL) for the treated eye (top) and untreated eye (bottom) from subject 13 at baseline (left), 6 months (center) and 12 months (right) post-treatment. Total area of the ONL is indicated.

FIG. 45 shows thickness maps of the photoreceptors inner segments for the treated eye (top) and untreated eye (bottom) from subject 13 at baseline (left), 6 months (center) and 12 months (right) post-treatment. Total area of the photoreceptors outer segments is indicated.

FIG. 46 shows thickness maps of the photoreceptors outer segments for the treated eye (top) and untreated eye (bottom) from subject 13 at baseline (left), 6 months (center) and 12 months (right) post-treatment. Total area of the photoreceptors outer segments is indicated.

FIG. 47 shows thickness maps of the RPE and drusen complex for the treated eye (top) and untreated eye (bottom) from subject 13 at baseline (left), 6 months (center) and 12 months (right) post-treatment. Total area of the RPE and drusen complex is indicated.

FIG. 48 shows total retina thickness maps for the treated eye (top) and untreated eye (bottom) from subject 14 at baseline (left) and 12 months (right) post-treatment. Average total thickness is indicated.

FIG. 49 shows thickness maps of the outer nuclear layer (ONL) for the treated eye (top) and untreated eye (bottom) from subject 14 at baseline (left) and 12 months (right) post-treatment. Total area of the ONL is indicated.

FIG. 50 shows thickness maps of the photoreceptors inner segments for the treated eye (top) and untreated eye (bottom) from subject 14 at baseline (left) and 12 months (right) post-treatment. Total area of the photoreceptors outer segments is indicated.

FIG. 51 shows thickness maps of the photoreceptors outer segments for the treated eye (top) and untreated eye (bottom) from subject 14 at baseline (left) and 12 months (right) post-treatment. Total area of the photoreceptors outer segments is indicated.

FIG. 52 shows thickness maps of the RPE and drusen complex for the treated eye (top) and untreated eye (bottom) from subject 14 at baseline (left) and 12 months (right) post-treatment. Total area of the RPE and drusen complex is indicated.

FIG. 53 shows baseline FA exam in subject 8, with massive fluorescein dye leaking into the vitreous cavity, which blocks the visibility of vascular perfusion during choroidal flush and arterial phase, suggesting the blood-retinal barrier breakdown and para-inflammation pre-existing within the eye. At 22 months post-transplant, FA exam showed clear choroidal and retinal vascular perfusion, there was no dye leaking into the vitreous cavity, indicating that OpRegen has restored the integrity of the broken BRB possibly through multiple mechanism of actions.

FIGS. 54A-54D show four cases with similar changes or improvement of FA imaging between baseline and post-transplant between 10.5 months and 22 months.

FIG. 55 shows drusen resolution started from graft area at superior (top left), then moved down cleaning up almost the entire posterior except a small elongated band remained at 8 months post-op (top, second from left, large circle). OCT imaging features are in concert with color fundus photography, at 5.5 months (top, second from right) and 8 months (bottom, second from right), compared to baseline (top right and bottom right), subRPE drusen was significant reduced or resolved.

FIG. 56A: FA showed significant reduced staining (drusen), yet, appeared to have membrane like veil that blurs the retina vascular architect. The pericyte reaction was visible. FIG. 56B: At 22 months on color fundus exam, the retinal tissue appears sharper compared to that on baseline. FIG. 56C shows at 11 months, graft continued to remodel the host retina after the large drusen resolute.

FIG. 57 provides the time course FA exams from early phase, mid-phase and late phase, demonstrating a significant improvement of retinal health with better visibility of vascular perfusion throughout, and reduced inflammation, retinal tissue appears very clean.

FIG. 58 shows OpRegen cell therapy in GA scar and ECM remodeling.

FIG. 59 shows OCT images of different forms of ECM remodeling.

FIGS. 60A and 60B show two tables illustrating the visual function in a cohort 4 subject by measuring changes in numbers of letters in an ETDRS test from baseline by 6M time. FIG. 60A represents the visual function of the treated eye and FIG. 60B represents the visual function of the fellow eye. Baseline is represented by 0, and positive number (also marked green) represent the number of letters gained from baseline. Negative number (also marked red) is represented by minus before the number, and represent the number of letters lost from baseline. For example, subject 13 (602) maintain steady BCVA improvement and gained 19 letters from baseline on his last visit.

The Figures provide various illustrations and examples of results that are surprising and unexpected. Embodiments relate to various methods that can include any of the assessments and assays discussed, set forth or for which data is presented in the Figures.

### DETAILED DESCRIPTION

Embodiments herein generally relate to methods, compositions of matter, and devices for treating diseases and illnesses of the eye, including retinal conditions such as macular degeneration.

In some embodiments, the compositions of matter, methods and devices can utilize product candidates that are allogeneic (“off-the-shelf”). For example, that can mean that the material is derived from cell lines, not from individual patients, facilitating large-scale production and lower production costs than patient-specific treatments.

The methods, device, compositions of matter, etc., can include those set forth in the accompanying Figures.

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. “Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a composition to accomplish a stated purpose relative to the absence of the composition (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug (e.g., the cells described herein) is an amount of the drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any composition described herein, the therapeutically effective amount can be initially determined from cell culture assays. Target concentrations will be those concentrations of active composition(s) (e.g., cell concentration or number) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring effectiveness of a composition and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the composition being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the composition. Thereafter, the dosage is increased by small increments until the optimum effect under circumstances is reached. Dosage amounts and intervals can be adjusted individually to provide levels of the administered composition effective for the particular clinical indication being treated. This will provide a therapeutic regimen that is commensurate with the severity of the individual's disease state.

“Co-administer” it is meant that a composition described herein is administered at the same time, just prior to, or just after the administration of one or more additional therapies. The compositions provided herein can be administered alone or can be coadministered to the patient. Coadministration is meant to include simultaneous or sequential administration of the compositions individually or in combination (more than one composition). Thus, the preparations can also be combined, when desired, with other active substances (e.g. to reduce metabolic degradation).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a composition as described herein (including embodiments and examples).

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions of the disclosure. One of skill in the art will recognize that other pharmaceutical excipients are useful in the present disclosure.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the “stem cells” refers to cells which are capable of remaining in an undifferentiated state (e.g., pluripotent or multipotent stem cells) for extended periods of time in culture until induced to differentiate into other cell types having a particular, specialized function (e.g., fully differentiated cells). In embodiments, “stem cells” include embryonic stem cells (ESCs), induced pluripotent stem cells (iPSCs), adult stem cells, mesenchymal stem cells and hematopoietic stem cells. In embodiments, RPE cells are generated from pluripotent stem cells (e.g., ESCs or iPSCs).

As used herein, “induced pluripotent stem cells” or “iPSCs” are cells that can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); IH Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis. In addition, iPSCs may be generated using non-integrating methods e.g., by using small molecules or RNA.

The term “embryonic stem cells” refers to embryonic cells that are capable of differentiating into cells of all three embryonic germ layers (i.e., endoderm, ectoderm and mesoderm), or remaining in an undifferentiated state. The phrase “embryonic stem cells” comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO 2006/040763) and embryonic germ (EG) cells which are obtained from the genital tissue of a fetus any time during gestation, preferably before 10 weeks of gestation. In embodiments, embryonic stem cells are obtained using well-known cell-culture methods. For example, human embryonic stem cells can be isolated from human blastocysts.

It is appreciated that commercially available stem cells can also be used in aspects and embodiments of the present disclosure. Human ES cells may be purchased from the NIH human embryonic stem cells registry, www.grants.nih.govstem_cells/ or from other hESC registries. Non-limiting examples of commercially available embryonic stem cell lines are HAD-C 102, ESI, BGO 1, BG02, BG03, BG04, CY12, CY30, CY92, CY1O, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WAO 1, UCSF4, NYUES 1, NYUES2, NYUES3, NYUES4, NYUESS, NYUES6, NYUES7, UCLA 1, UCLA 2, UCLA 3, WA077 (H7), WA09 (H9), WA 13 (H13), WA14 (H14), HUES 62, HUES 63, HUES 64, CT I, CT2, CT3, CT4, MA135, Eneavour-2, WIBR 1, WIBR2, WIBR3, WIBR4, WIBRS, WIBR6, HUES 45, Shef 3, Shef 6, BINhem19, BJNhem20, SAGO 1, SAOO1.

The term “retinal pigment epithelium” or “RPE,” also known as “pigmented layer of retina,” refers to the pigmented layer of cells outside the retina. The RPE layer is located between the Bruch's membrane (choroid inner border) and the photoreceptors. The RPE is an intermediate for supplying nutrients to the retina, and assists in numerous functions, including retina development, absorption of light, secretion of growth factors, and mediating the immune response of the eye. Dysfunction of the RPE may lead to vision loss or blindness in conditions including retinitis pigmentosa, diabetic retinopathy, West Nile virus, and macular degeneration.

The terms “disease” or “condition” refer to a state of being or health status of a patient or subject capable of being treated with the compositions or methods provided herein. Age-related Macular Degeneration or AMD is a progressive chronic disease of the central retina and a leading cause of vision loss worldwide. Most visual loss occurs in the late stages of the disease due to one of two processes: neovascular (“wet”) AMD and geographic atrophy (GA, “dry”). In GA, progressive atrophy of the retinal pigment epithelium, choriocapillaris, and photoreceptors occurs. The dry form of AMD is more common (85-90% of all cases), but may progress to the “wet” form, which, if left untreated, leads to rapid and severe vision loss. The estimated prevalence of AMD is 1 in 2,000 people in the US and other developed countries. This prevalence is expected to increase together with the proportion of elderly in the general population. The risk factors for the disease include both environmental and genetic factors. The pathogenesis of the disease involves abnormalities in four functionally interrelated tissues, i.e., retinal pigment epithelium (RPE), Bruch's membrane, choriocapillaries and photoreceptors. However, impairment of RPE cell function is an early and crucial event in the molecular pathways leading to clinically relevant AMD changes. There is currently no approved treatment for dry-AMD. Prophylactic measures include vitamin/mineral supplements. These reduce the risk of developing wet AMD but do not affect the development of progression of geographic atrophy (GA).

A non-limiting list of diseases for which the effects of treatment may be measured in accordance with the methods provided herein comprises retinitis pigmentosa, lebers congenital amaurosis, hereditary or acquired macular degeneration, age related macular degeneration (AMD), geographic atrophy (GA), Best disease, retinal detachment, gyrate atrophy, choroideremia, pattern dystrophy as well as other dystrophies of the RPE, Stargardt disease, RPE and retinal damage due to damage caused by any one of photic, laser, inflammatory, infectious, radiation, neo vascular or traumatic injury, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, pathologic myopia, retinitis pigmentosa, and macular degeneration. In embodiments, the disease is dry AMD. In embodiments, the disease is GA.

“Geographic atrophy” or “GA” or “atrophic retina,” also known as atrophic age-related macular degeneration (AMD) or advanced dry AMD, is an advanced form of age-related macular degeneration that can result in the progressive and irreversible loss of retina (photoreceptors, retinal pigment epithelium, choriocappillaris), which may lead to a loss of visual function over time.

In embodiments, the RPE defects may result from one or more of: advanced age, cigarette smoking, unhealthy body weight, low intake of antioxidants, or cardiovascular disorders. In other embodiments, the RPE defects may result from a congenital abnormality. “Retinal pigment epithelium cells”, “RPE cells”, “RPEs”, which may be used interchangeably as the context allows, refers to cells of a cell type that is for example, functionally, epigenetically, or by expression profile similar to that of native RPE cells which form the pigment epithelium cell layer of the retina (e.g., upon transplantation, administration or delivery within an eye, they exhibit functional activities similar to those of native RPE cells).

As used herein, the term “OpRegen” refers to a lineage-restricted human RPE cell line. The RPE cells are derived under differentiation media supplemented with Activin A, a transforming growth factor beta (TGF-b) family and nicotinamide to enrich the RPE population. OpRegen is a single cell suspension formulated either in ophthalmic Balanced Salt Solution (BSS Plus) or as a ready to administer (RTA) thaw and inject (TAI) formulation in CryoStor®5.

### Methods of Treatment

Embodiments herein generally relate to methods, compositions of matter, and devices for treating diseases and illnesses of the eye, including retinal conditions such as macular degeneration.

Thus, in an aspect is provided a method of treating or slowing the progression of retinal disease or disorder as set forth, described or illustrated herein.

According to some embodiments, treating or slowing the progression of a retinal disease can be demonstrated by microperimetry to assess recovery of vision. Microperimetry is one of the tools that can be used to measure or assess vision functions with high resolution mapping of the visual sensitivity area. Microperimetry allows for locating this specific area of vision or impaired vision on the retina and can “bridge over the gap” between anatomical and clinical changes with good correlation between these two important parameters (anatomical defect vs. visual impairment).

According to other embodiments, microperimetry-assessed recovery of vision comprises demonstrating that administration of the RPE cells comprises an improved microperimetry assessment compared to a baseline microperimetry assessment. According to other embodiments, microperimetry-assessed recovery of vision comprises demonstrating that administration of the RPE cells comprises a preserved microperimetry assessment compared to baseline and the fellow/untreated eye.

According to certain embodiments, treating or slowing the progression of a retinal disease comprises a reduction in rate of GA lesion growth relative to a baseline or fellow eye of between about 5% and about 20% at one year after administration of RPE cells. In embodiments, treating or slowing the progression of a retinal disease comprises a reduction in rate of GA lesion growth relative to a baseline or fellow eye of between about 5% and about 50% at one year after administration. In embodiments, treating or slowing the progression of a retinal disease comprises a reduction in rate of GA lesion growth relative to a baseline or fellow eye of between about 5% and about 25% at one year after administration. In embodiments, treating or slowing the progression of a retinal disease comprises a reduction in rate of GA lesion growth relative to a baseline or fellow eye of between about 5% and about 100% at one year after administration. In embodiments, treating or slowing the progression of a retinal disease comprises a reduction in rate of GA lesion growth relative to a baseline or fellow eye of between about 5% and about 10% at one year after administration. The amount may be any value or subrange within the recited ranges, including endpoints.

According to some embodiments, treating or slowing the progression of a retinal disease comprises one or more of: a stable best-corrected visual acuity (BCVA); no deterioration in low luminance test performance; or no deterioration in microperimetry sensitivity; or no deterioration in reading speed. In embodiments, comparison is to age-matched, sex-matched control. In embodiments, comparison is to a baseline. In embodiments, comparison is to a fellow eye. In embodiments, the comparison is at a time period between about one week and about 5 years. In embodiments, the comparison is at about one month. In embodiments, the comparison is at about three months. In embodiments, the comparison is at about six months. In embodiments, the comparison is at about one year. The time period may be any value or subrange within the recited ranges, including endpoints.

According to some embodiments, a pharmaceutical composition for treating or slowing the progression of a retinal disease or disorder comprising as an active substance about between about 25,000 and about 1,000,000 RPE cells is presented. In embodiments, the composition comprises between about 50,000 and about 500,000 RPE cells. In embodiments, the composition comprises between about 100,000 and about 500,000 RPE cells. In embodiments, the composition comprises between about 250,000 and about 500,000 RPE cells. In embodiments, the composition comprises between about 50,000 and about 400,000 RPE cells. In embodiments, the composition comprises between about 50,000 and about 300,000 RPE cells. In embodiments, the composition comprises between about 50,000 and about 250,000 RPE cells. In embodiments, the composition comprises between about 50,000 and about 200,000 RPE cells. The amount may be any value or subrange within the recited ranges, including endpoints.

In some embodiments, the method comprises administering a cell therapeutic agent to a subject in need thereof, wherein the cell therapeutic agent is capable of restoring retinal structure of retinal disease.

Cell Therapeutic Agents

In some aspects the present disclosure is drawn cell therapeutic agents comprising retinal pigment epithelial (RPE) cells derived from pluripotent cells. Such cell therapeutic agents include, but are not intended to be limited to, OpRegen.

According to some embodiments, the RPE cells express at least one, two, three, four or five markers of mature RPE cells. According to some embodiments, the RPE cells express between at least two to at least ten or at least two to at least thirty markers of mature RPE cells. Such markers include, but are not limited to CRALBP, RPE65, PEDF, PMEL17, bestrophin 1 and tyrosinase. Optionally, the RPE cell may also express a marker of a RPE progenitor (e.g., MITF). In other embodiments, the RPE cells express PAX-6. In other embodiments, the RPE cells express at least one marker of a retinal progenitor cell including, but not limited to Rx, OTX2 or SIX3. Optionally, the RPE cells may express SIX6 and/or LHX2.

According to some embodiments, RPE cells are OpRegen® cells.

As used herein the phrase “markers of mature RPE cells” refers to antigens (e.g., proteins) that are elevated (e.g., at least 2-fold, at least 5-fold, at least 10-fold) in mature RPE cells with respect to non RPE cells or immature RPE cells.

As used herein the phrase “markers of RPE progenitor cells” refers to antigens (e.g., proteins) that are elevated (e.g. at least 2-fold, at least 5-fold, at least 10-fold) in RPE progenitor cells when compared with non RPE cells.

According to other embodiments, the RPE cells have a morphology similar to that of native RPE cells which form the pigment epithelium cell layer of the retina. For example, the cells may be pigmented and have a characteristic polygonal shape.

According to some embodiments, the RPE cells are generated from pluripotent stem cells (e.g., ESCs or iPSCs).

Induced pluripotent stem cells (iPSCs) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis. In addition, iPSCs may be generated using non-integrating methods e.g., by using small molecules or RNA.

Human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by a procedure in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells, see Reubinoff et al. Nat Biotechnol 2000, May: 18(5): 559; Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

In addition, ES cells can be obtained from other species, including mouse (Mills and Bradley, 2001), golden hamster [Doetschman et al., 1988, Dev Biol. 127: 224-7], rat [Iannaccone et al., 1994, Dev Biol. 163: 288-92], rabbit [Giles et al. 1993, Mol Reprod Dev. 36: 130-8; Graves & Moreadith, 1993, Mol Reprod Dev. 1993, 30 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG (embryonic germ) cells are prepared from the primordial germ cells obtained from fetuses of about 8-11 weeks of gestation (in the case of a human fetus) using laboratory techniques known to anyone skilled in the arts. The genital ridges are dissociated and cut into small portions which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Yet another method for preparing ES cells is by parthenogenesis. The embryo is also not destroyed in the process.

ES culturing methods may include the use of feeder cell layers which secrete factors needed for stem cell proliferation, while at the same time, inhibiting their differentiation. The culturing is typically effected on a solid surface, for example a surface coated with gelatin or vimentin. Exemplary feeder layers include human embryonic fibroblasts, adult fallopian epithelial cells, primary mouse embryonic fibroblasts (PMEF), mouse embryonic fibroblasts (MEF), murine fetal fibroblasts (MFF), human embryonic fibroblast (HEF), human fibroblasts obtained from the differentiation of human embryonic stem cells, human fetal muscle cells (HFM), human fetal skin cells (HFS), human adult skin cells, human foreskin fibroblasts (HFF), human umbilical cord fibroblasts, human cells obtained from the umbilical cord or placenta, and human marrow stromal cells (hMSCs). Growth factors may be added to the medium to maintain the ESCs in an undifferentiated state. Such growth factors include bFGF and/or TGF. In another embodiment, agents may be added to the medium to maintain the hESCs in a naive undifferentiated state—see for example Kalkan et al., 2014, Phil. Trans. R. Soc. B, 369: 20130540.

Human umbilical cord fibroblasts may be expanded in Dulbecco's Modified Eagle's Medium (e.g. DMEM, SH30081.01, Hyclone) supplemented with human serum (e.g. 20%) and glutamine. Preferably the human cord cells are irradiated. This may be effected using methods known in the art (e.g. Gamma cell, 220 Exel, MDS Nordion 3,500-7500 rads). Once sufficient cells are obtained, they may be frozen (e.g. cryopreserved). For expansion of ESCs, the human cord fibroblasts are typically seeded on a solid surface (e.g. T75 or T 175 flasks) optionally coated with an adherent substrate such as gelatin (e.g. recombinant human gelatin (RhG 100-001, Fibrogen) or human Vitronectin or Laminin 521 (Bio lamina) at a concentration of about 25,000-100,000 cells/cm2 in DMEM (e.g. SH30081.01, Hyclone) supplemented with about 20% human serum (and glutamine). hESCs are typically plated on top of the feeder cells 1-4 days later in a supportive medium (e.g. NUTRISTEM® or NUT(+) with human serum albumin). Additional factors may be added to the medium to prevent differentiation of the ESCs such as bFGF and TGFI3. Once a sufficient amount of hESCs are obtained, the cells may be mechanically disrupted (e.g. by using a sterile tip or a disposable sterile stem cell tool; 14602 Swemed). Alternatively, the cells may be removed by enzymatic treatment (e.g. collagenase A, or TrypLE Select). This process may be repeated several times to reach the necessary amount of hESC. According to some embodiments, following the first round of expansion, the hESCs are removed using TrypLE Select and following the second round of expansion, the hESCs are removed using collagenase A.

The ESCs may be expanded on feeders prior to the differentiation step. Nonlimiting examples of feeder layer based cultures are described herein above. The expansion is typically effected for at least two days, three days, four days, five days, six days, seven days, eight days, nine days, or ten days. The expansion is effected for at least 1 passage, at least 2 passages, at least 3 passages, at least 4 passages, at least 5 passages, at least 6 passages, at least 7 passages, at least 8 passages, at least 9 passages or at least 10 passages. In some embodiments, the expansion is effected for at least 2 passages to at least 20 passages. In other embodiments, the expansion is effected for at least 2 to at least 40 passages. Following expansion, the pluripotent stem cells (e.g. ESCs) are subjected to directed differentiation using a differentiating agent.

Feeder cell free systems have also been used in ES cell culturing, such systems utilize matrices supplemented with serum replacement, cytokines and growth factors (including IL6 and soluble IL6 receptor chimera) as a replacement for the feeder cell layer. Stem cells can be grown on a solid surface such as an extracellular matrix (e.g., MATRIGELR™, laminin or vitronectin) in the presence of a culture medium—for example the Lonza L7 system, mTeSR, StemPro, XFKSR, E8, NUTRISTEM®). Unlike feeder-based cultures which require the simultaneous growth of feeder cells and stem cells and which may result in mixed cell populations, stem cells grown on feeder-free systems are easily separated from the surface. The culture medium used for growing the stem cells contains factors that effectively inhibit differentiation and promote their growth such as MEF-conditioned medium and bFGF.

In some embodiments, following expansion, the pluripotent ESCs are subjected to directed differentiation on an adherent surface (without intermediate generation of spheroid or embyroid bodies). See, for example, international patent application publication No. WO 2017/072763, incorporated by reference herein in its entirety.

Thus, according to an aspect of the present disclosure, at least 50%, 55%, 60%, 65%, 70%, 75%, 80%, 85%, 90%, 91%, 92%, 93%, 94%, 95%, 96%, 97%, 98%, 99% or 100% of the cells which are subjected to directed differentiation on the adherent surface are undifferentiated ESCs and express markers of pluripotency. For example, at least 50%, 55%, 60%, 65%, 70%, 75%, 80%, 85%, 90%, 91%, 92%, 93%, 94%, 95%, 96%, 97%, 98%, 99% or 100% of the cells are Oct4±TRA-1-60+. The non-differentiated ESCs may express other markers of pluripotency, such as NANOG, Rex-1, alkaline phosphatase, Sox2, TDGF-beta, SSEA-3, SSEA-4 and/or TRA-1-81.

In one exemplary differentiation protocol, the non-differentiated embryonic stem cells are differentiated towards the RPE cell lineage on an adherent surface using a first differentiating agent and then further differentiated towards RPE cells using a member of the transforming growth factor-B (TGFB) superfamily, (e.g. TGF 1, TGF2, and TGF 3 subtypes, as well as homologous ligands including activin (e.g., activin A, activin B, and activin AB), nodal, anti-mullerian hormone (AMH), some bone morphogenetic proteins (BMP), e.g. BMP2, BMP3, BMP4, BMP5, BMP6, and BMP7, and growth and differentiation factors (GDF)). According to a specific embodiment, the member of the transforming growth factor-B (TGFB) superfamily is activin A—e.g. between 20-200 ng/ml e.g. 100-180 ng/ml.

According to some embodiments, the first differentiating agent is nicotinamide (NA) used at concentrations of between about 1-100 mM, 5-50 mM, 5-20 mM, and for example, 10 mM. According to other embodiments, the first differentiating agent is 3-aminobenzmine.

NA, also known as “niacinamide”, is the amide derivative form of Vitamin B3 (niacin) which is thought to preserve and improve beta cell function. NA has the chemical formula C6H6N20. NA is essential for growth and the conversion of foods to energy, and it has been used in arthritis treatment and diabetes treatment and prevention.

According to some embodiments, the nicotinamide is a nicotinamide derivative or a nicotinamide mimic. The term “derivative of nicotinamide (NA)” as used herein denotes a compound which is a chemically modified derivative of the natural NA. In one embodiment, the chemical modification may be a substitution of the pyridine ring of the basic NA structure (via the carbon or nitrogen member of the ring), via the nitrogen or the oxygen atoms of the amide moiety. When substituted, one or more hydrogen atoms may be replaced by a substituent and/or a substituent may be attached to a N atom to form a tetravalent positively charged nitrogen. Thus, the nicotinamide of the present invention includes a substituted or non-substituted nicotinamide. In another embodiment, the chemical modification may be a deletion or replacement of a single group, e.g. to form a thiobenzamide analog of NA, all of which being as appreciated by those versed in organic chemistry. The derivative in the context of the invention also includes the nucleoside derivative of NA (e.g. nicotinamide adenine). A variety of derivatives of NA are described, some also in connection with an inhibitory activity of the PDE4 enzyme (WO 03/068233; WO 02/060875; GB2327675A), or as VEGF-receptor tyrosine kinase inhibitors (WOO 1/55114). For example, the process of preparing 4-aryl-nicotinamide derivatives (WO 05/014549). Other exemplary nicotinamide derivatives are disclosed in WOO 1/55114 and EP2128244.

Nicotinamide mimics include modified forms of nicotinamide, and chemical analogs of nicotinamide which recapitulate the effects of nicotinamide in the differentiation and maturation of RPE cells from pluripotent cells. Exemplary nicotinamide mimics include benzoic acid, 3-aminobenzoic acid, and 6-aminonicotinamide. Another class of compounds that may act as nicotinamide mimics are inhibitors of poly(ADP-ribose) polymerase (PARP). Exemplary PARP inhibitors include 3-aminobenzamide, Iniparib (BSI 201), Olaparib (AZD-2281), Rucaparib (AG014699, PF-01367338), Veliparib (ABT-888), CEP 9722, MK 4827, and BMN-673.

Additional contemplated differentiation agents include for example noggin, antagonists of Wnt (Dkkl or IWR1e), nodal antagonists (Lefty-A), retinoic acid, taurine, GSK3b inhibitor (CHIR99021) and notch inhibitor (DAPT).

According to certain embodiments, the differentiation is effected as follows: (a) culture of ESCs in a medium comprising a first differentiating agent (e.g. nicotinamide); and (b) culture of cells obtained from step a) in a medium comprising a member of the TGFB superfamily (e.g. activin A) and the first differentiating agent (e.g. nicotinamide).

Step (a) may be effected in the absence of the member of the TGFI3 superfamily (e.g. activin A).

In some embodiments, the medium in step (a) is completely devoid of a member of the TGFI3 superfamily. In other embodiments, the level of TGFI3 superfamily member in the medium is less than 20 ng/ml, 10 ng/ml, 1 ng/ml or even less than 0.1 ng/ml.

The above described protocol may be continued by culturing the cells obtained in step (b) in a medium comprising the first differentiating agent (e.g. nicotinamide), but devoid of a member of the TGFI3 superfamily (e.g. activin A). This step is referred to herein as step (b*).

The above described protocol is now described in further detail, with additional embodiments. Step (a): The differentiation process is started once sufficient quantities of ESCs are obtained. The cells may be removed from the cell culture (e.g. by using collagenase A, dispase, TrypLE select, EDTA) and plated onto a non-adherent substrate (e.g. cell culture plate such as Hydrocell or an agarose-coated culture dish, or petri bacteriological dishes) in the presence of nicotinamide (and the absence of activin A). Exemplary concentrations of nicotinamide are between 0.01-100 mM, 0.1-100 mM, 0.1-50 mM, 5-50 mM, 5-20 mM, and 10 mM. Once the cells are plated onto the non-adherent substrate (e.g. cell culture plate), the cell culture may be referred to as a cell suspension, preferably free-floating clusters in a suspension culture, i.e. aggregates of cells derived from human embryonic stem cells (hESCs). The cell clusters do not adhere to any substrate (e.g. culture plate, carrier). Sources of free floating stem cells were previously described in WO 06/070370, which is herein incorporated by reference in its entirety. This stage may be effected for a minimum of 1 day, more preferably two days, three days, 1 week or even 14 days. Preferably, the cells are not cultured for more than 3 weeks in suspension together with the nicotinamide e.g. between 0.01-100 mM, 0.1-100 mM, 0.1-50 mM, 5-50 mM, 5-20 mM, e.g. 10 mM (and in the absence of activin A). In one embodiment, the cells are cultured for 6-8 days in suspension together with the nicotinamide e.g. between 0.01-100 mM, 0.1-100 mM, 0.1-50 mM, 5-50 mM, 5-20 mM, e.g. 10 mM (and in the absence of activin A).

According to some embodiments, when the cells are cultured on the non-adherent substrate, e.g. cell culture plates, the atmospheric oxygen conditions are 20%. However, manipulation of the atmospheric oxygen conditions is also contemplated such that the atmospheric oxygen percent is less than about 20%, 15%, 10%, 9%, 8%, 7%, 6% or even less than about 5% (e.g. between 1%-20%, 1%-10% or 0-5%). According to other embodiments, the cells are cultured on the non-adherent substrate initially under normal atmospheric oxygen conditions and then lowered to less than normal atmospheric oxygen conditions.

Examples of non-adherent cell culture plates include those manufactured by Nunc (e.g. Hydrocell Cat No. 174912), etc.

Typically, the clusters comprise at least about 50 to 500,000, 50 to 100,000, 50 to 50,000, 50 to 10,000, 50 to 5000, or 50 to 1000 cells. According to one embodiment, the cells in the clusters are not organized into layers and form irregular shapes. In one embodiment, the clusters are substantially devoid of pluripotent embryonic stem cells. In another embodiment, the clusters comprise small amounts of pluripotent embryonic stem cells (e.g. no more than 5%, or no more than 3% (e.g. 0.01-2.7%) cells that co-express OCT4 and TRA-1-60 at the protein level). Typically, the clusters comprise cells that have been partially differentiated under the influence of nicotinamide. Such cells primarily express neural and retinal precursor markers such as PAX6, Rax, Six3 and/or CHX10.

The clusters may be dissociated using enzymatic or non-enzymatic methods (e.g., mechanical) known in the art. According to some embodiments, the cells are dissociated such that they are no longer in clusters—e.g. aggregates or clumps of 2-100,000 cells, 2-50,000 cells, 2-10,000 cells, 2-5000 cells, 2-1000 cells, 2-500 cells, 2-100 cells, 2-50 cells. According to a particular embodiment, the cells are in a single cell suspension.

The cells (e.g. dissociated cells) can then be plated on an adherent substrate and cultured in the presence of nicotinamide e.g. between 0.01-100 mM, 0.1-100 mM, 0.1-50 mM, 5-50 mM, 5¬20 mM, and for example, 10 mM (and in the absence of activin A). The concentration may be any value or subrange within the recited ranges, including endpoints. This stage may be effected for a minimum of 1 day, more preferably two days, three days, 1 week or even 14 days. Preferably, the cells are not cultured for more than 3 weeks in the presence of nicotinamide (and in the absence of activin). In an exemplary embodiment, this stage is effected for 6-7 days.

According to other embodiments, when the cells are cultured on the adherent substrate e.g. laminin, the atmospheric oxygen conditions are 20%. They may be manipulated such that the atmospheric oxygen percentage is less than about 20%, 15%, 10%, more preferably less than about 9%, less than about 8%, less than about 7%, less than about 6% and more preferably about 5% (e.g. between 1%-20%, 1%-10% or 0-5%). The amount may be any value or subrange within the recited ranges, including endpoints.

According to some embodiments, the cells are cultured on the adherent substrate initially under normal atmospheric oxygen conditions and subsequently the oxygen is lowered to less than normal atmospheric oxygen conditions.

Examples of adherent substrates or a mixture of substances could include but are not limited to fibronectin, laminin, polyD-lysine, collagen and gelatin.

Step (b): Following the first stage of directed differentiation, (step a; i.e. culture in the presence of nicotinamide (e.g. between 0.01-100 mM, 0.1-100 mM, 0.1-50 mM, 5-50 mM, 5-20 mM, e.g. 10 mM), the partially-differentiated cells may then be subjected to a further stage of differentiation on an adherent substrate by culturing in the presence of activin A (e.g. 0.01¬1000 ng/ml, 0.1-200 ng/ml, 1-200 ng/ml—for example 140 ng/ml, 150 ng/ml, 160 ng/ml or 180 ng/ml). Thus, activin A may be added at a final molarity of 0.1 pM-10 nM, 10 pM-10 nM, 0.1 nM-10 nM, 1 nM-10 nM, for example 5.4 nM. The concentration may be any value or subrange within the recited ranges, including endpoints.

Nicotinamide may be added at this stage as well (e.g. between 0.01-100 mM, 0.1-100 mM, 0.1¬50 mM, 5-50 mM, 5-20 mM, e.g. 10 mM). The concentration may be any value or subrange within the recited ranges, including endpoints. This stage may be effected for 1 day to 10 weeks, 3 days to 10 weeks, 1 week to 10 weeks, one week to eight weeks, one week to four weeks, for example for at least one day, at least two days, at least three days, at least 5 days, at least one week, at least 9 days, at least 10 days, at least two weeks, at least three weeks, at least four weeks, at least five weeks, at least six weeks, at least seven weeks, at least eight weeks, at least nine weeks, at least ten weeks. The time period may be any value or subrange within the recited ranges, including endpoints.

According to some embodiments, this stage is effected for about eight days to about two weeks. This stage of differentiation may be effected at low or normal atmospheric oxygen conditions, as detailed herein above.

Step (b*): Following the second stage of directed differentiation (i.e. culture in the presence of nicotinamide and activin A on an adherent substrate; step (b), the further differentiated cells are optionally subjected to a subsequent stage of differentiation on the adherent substrate-culturing in the presence of nicotinamide (e.g. between 0.01-100 mM, 0.1-100 mM, 0.1-50 mM, 5-50 mM, 5-20 mM, e.g. 10 mM), in the absence of activin A. The concentration may be any value or subrange within the recited ranges, including endpoints. This stage may be effected for at least one day, 2, days, 5 days, at least one week, at least two weeks, at least three weeks or even four weeks. This stage of differentiation may also be carried out at low or normal atmospheric oxygen conditions, as detailed herein above.

The basic medium in which the ESCs are differentiated is any known cell culture medium known in the art for supporting cell growth in vitro, typically, a medium comprising a defined base solution, which includes salts, sugars, amino acids and any other nutrients required for the maintenance of the cells in the culture in a viable state. According to a specific embodiment, the basic medium is not a conditioned medium. Non-limiting examples of commercially available basic media that may be utilized in accordance with the invention comprise NUTRISTEM® (without bFGF and TGF for ESC differentiation, with bFGF and TGF for ESC expansion), NEUROBASAL™, KO-DMEM, DMEM, DMEM/F12, CELLGRO™ Stem Cell Growth Medium, or X-VIVO™. The basic medium may be supplemented with a variety of agents as known in the art dealing with cell cultures. The following is a non-limiting reference to various supplements that may be included in the culture to be used in accordance with the present disclosure: serum or with a serum replacement containing medium, such as, without being limited thereto, knock out serum replacement (KOSR), NUTRIDOMA-CS, TCH™, N2, N2 derivative, or B27 or a combination; an extracellular matrix (ECM) component, such as, without being limited thereto, fibronectin, laminin, collagen and gelatin. The ECM may then be used to carry the one or more members of the TGFI3 superfamily of growth factors; an antibacterial agent, such as, without being limited thereto, penicillin and streptomycin; and non-essential amino acids (NEAA), neurotrophins which are known to play a role in promoting the survival of SCs in culture, such as, without being limited thereto, BDNF, NT3, NT4.

According to some embodiments, the medium used for differentiating the ESCs is NUTRISTEM® medium (Biological Industries, 06-5102-01-1A).

According to some embodiments, differentiation and expansion of ESCs is effected under xeno free conditions. According other embodiments, the proliferation/growth medium is substantially devoid of xeno contaminants i.e., free of animal derived components such as serum, animal derived growth factors and albumin. Thus, according to these embodiments, the culturing is performed in the absence of xeno contaminants. Other methods for culturing ESCs under xeno free conditions are provided in U.S. Patent Application No. 20130196369, the contents of which are incorporated herein by reference in its entirety.

The preparations comprising RPE cells may be prepared in accordance with Good Manufacturing Practices (GMP) (e.g., the preparations are GMP-compliant) and/or current Good Tissue Practices (GTP) (e.g., the preparations may be GTP-compliant).

During differentiation steps, the embryonic stem cells may be monitored for their differentiation state. Cell differentiation can be determined upon examination of cell or tissue-specific markers which are known to be indicative of differentiation.

Tissue/cell specific markers can be detected using immunological techniques well known in the art [Thomson J A et al., (1998). Science 282: 1145-7]. Examples include, but are not limited to, flow cytometry for membrane-bound or intracellular markers, immunohistochemistry for extracellular and intracellular markers and enzymatic immunoassay, for secreted molecular markers.

Following the stages of differentiation described herein above, a mixed cell population can be obtained comprising both pigmented and non-pigmented cells. According to this aspect, the cells of the mixed cell population are removed from the plate. In some embodiments, this is effected enzymatically (e.g. using trypsin, (TrypLE Select); see for example, international patent application publication No. WO 2017/021973, incorporated by reference herein in its entirety). According to this aspect of the present invention, at least 10%, 20%, 30%, at least 40%, at least 50%, at least 60%, at least 70% of the cells which are removed from the culture (and subsequently expanded) are non-pigmented cells. In other embodiments, this is effected mechanically—e.g. using a cell scraper. In yet other embodiments, this is effected chemically (e.g., by EDTA). Combinations of enzymatic and chemical treatment are also contemplated. For example, EDTA and enzymatic treatments can be used. Furthermore, at least 10%, 20% or even 30% of the cells which are removed from the culture (and subsequently expanded) may be pigmented cells.

According to an aspect of the present disclosure, at least 50%, 60%, 70%, 80%, 90%, 95%, 100% of all the cells in the culture are removed and subsequently expanded.

Expansion of the mixed population of cells may be effected on an extra cellular matrix, e.g. gelatin, collagen I, collagen IV, laminin (e.g. laminin 521), fibronectin and poly-D-lysine. For expansion, the cells may be cultured in serum-free KOM, serum comprising medium (e.g. DMEM with 20% human serum) or NUTRISTEM® medium (06-5102-01-1A, Biological Industries). Under these culture conditions, after passaging under suitable conditions, the ratio of pigmented cells to non-pigmented cells increases such that a population of purified RPE cells is obtained. Such cells show the characteristic polygonal shape morphology and pigmentation of RPE cells.

In one embodiment, the expanding is effected in the presence of nicotinamide (e.g. between 0.01-100 mM, 0.1-100 mM, 0.1-50 mM, 5-50 mM, 5-20 mM, e.g. 10 mM), and in the absence of activin A. The concentration may be any value or subrange within the recited ranges, including endpoints.

The mixed population of cells may be expanded in suspension (with or without a micro-carrier) or in a monolayer. The expansion of the mixed population of cells in monolayer cultures or in suspension culture may be modified to large scale expansion in bioreactors or multi/hyper stacks by methods well known to those versed in the art.

According to some embodiments, the expansion phase is effected for at least one to 20 weeks, at least 2 weeks, at least 3 weeks, at least 4 weeks, at least 5 weeks, at least 6 weeks, at least 7 weeks, at least 8 weeks, at least 9 weeks or even 10 weeks. Preferably, the expansion phase is effected for 1 week to 10 weeks, more preferably 2 weeks to 10 weeks, more preferably, 3 weeks to 10 weeks, more preferably 4 weeks to 10 weeks, or 4 weeks to 8 weeks. The time period may be any value or subrange within the recited ranges, including endpoints.

According to still other embodiments, the mixed population of cells are passaged at least 1 time during the expansion phase, at least twice during the expansion phase, at least three times during the expansion phase, at least four times during the expansion phase, at least five times during the expansion phase, or at least six times during the expansion phase.

When cells are collected enzymatically, it is possible to continue the expansion for more than 8 passages, more than 9 passages and even more than 10 passages (e.g. 11-15 passages). The number of total cell doublings can be increased to greater than 30, e.g. 31, 32, 33, 34 or more. (See international patent application publication number WO 2017/021973, incorporated herein by reference in its entirety).

The population of RPE cells generated according to the methods described herein may be characterized according to a number of different parameters. Thus, for example, the RPE cells obtained may be polygonal in shape and pigmented.

It will be appreciated that the cell populations and cell compositions disclosed herein are generally devoid of undifferentiated human embryonic stem cells. According to some embodiments, less than 1:250,000 cells are Oct4+TRA-1-60+ cells, as measured for example by FACS. The cells may also have down regulated (by more than 5,000 fold) expression of GDF3 or TDGF as measured by PCR. The RPE cells of this aspect, do not substantially express embryonic stem cell markers. Said one or more embryonic stem cell markers may comprise OCT-4, NANOG, Rex-1, alkaline phosphatase, Sox2, TDGF-beta, SSEA-3, SSEA-4, TRA-1-60, and/or TRA-1-81.

The therapeutic RPE cell preparations may be substantially purified, with respect to non-RPE cells, comprising at least about 75%, 80%, 85%, 90%, 91%, 92%, 93%, 94%, 95%, 96%, 97%, 98%, 99%, or 100% RPE cells. The RPE cell preparations may be essentially free of non-RPE cells or consist of RPE cells. For example, the substantially purified preparation of RPE cells may comprise less than about 25%, 20%, 15%, 10%, 9%, 8%, 7%, 6%, 5%, 4%, 3%, 2%, or 1% non-RPE cell type. For example, the RPE cell preparation may comprise less than about 25%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% non-RPE cells.

The RPE cell preparations may be substantially pure, both with respect to non-RPE cells and with respect to RPE cells of other levels of maturity. The preparations may be substantially purified, with respect to non-RPE cells, and enriched for mature RPE cells. For example, in RPE cell preparations enriched for mature RPE cells, at least about 30%, 40%, 45%, 50%, 55%, 60%, 65%, 70%, 75%, 80%, 85%, 90%, 91%, 92%, 93%, 94%, 95%, 96%, 97%, 98%, 99%, 99%, or 100% of the RPE cells are mature RPE cells. The preparations may be substantially purified, with respect to non-RPE cells, and enriched for differentiated RPE cells rather than mature RPE cells. For example, at least about 30%, 40%, 45%, 50%, 55%, 60%, 65%, 70%, 75%, 80%, 85%, 90%, 91%, 92%, 93%, 94%, 95%, 96%, 97%, 98%, 99%, or 100% of the RPE cells may be differentiated RPE cells rather than mature RPE cells.

The preparations described herein may be substantially free of bacterial, viral, or fungal contamination or infection, including but not limited to the presence of HIV I, HIV 2, HBV, HCV, HAV, CMV, HTLV 1, HTLV 2, parvovirus B19, Epstein-Barr virus, or herpesvirus 1 and 2, SV40, HHVS, 6, 7, 8, CMV, polyoma virus, HPV, Enterovirus. The preparations described herein may be substantially free of mycoplasma contamination or infection.

Another way of characterizing the cell populations disclosed herein is by marker expression. Thus, for example, at least 80%, 85%, 90%, 95% or 100% of the cells may express Bestrophin 1, as measured by immunostaining. According to one embodiment, between 80-100% of the cells express bestrophin 1.

According to other embodiments, at least 80%, 85%, 87%, 89%, 90%, 95%, 97% or 100% of the cells express Microphthalmia-associated transcription factor (MITF), as measured by immunostaining. For example, between 80-100% of the cells express MITF.

According to other embodiments, at least 80%, 85%, 87%, 89%, 90%, 95%, 97% or 100% of the cells express both Microphthalmia-associated transcription factor (MITF) and bestrophin 1, as measured by immunostaining. For example, between 80-100% of the cells co-express MITF and bestrophin 1.

According to other embodiments, at least 80%, 85%, 87%, 89%, 90%, 95%, 97% or 100% of the cells express both Microphthalmia-associated transcription factor (MITF) and ZO-1, as measured by immunostaining. For example, between 80-100% of the cells co-express MITF and ZO-1.

According to other embodiments, at least 80%, 85%, 87%, 89%, 90%, 95%, 97% or 100% of the cells express both ZO-1 and bestrophin 1, as measured by immunostaining.

For example, between 80-100% of the cells co-express ZO-1 and bestrophin 1.

According to another embodiment, at least 50%, 60% 70% 80%, 85%, 87%, 89%, 90%, 95%, 97% or 100% of the cells express paired box gene 6 (PAX-6) as measured by immunostaining or FACS. For example, at least between 50% and 100% of the cells express paired box gene 6 (PAX-6).

According to another embodiment, at least 80%, 85%, 87%, 89%, 90%, 95%, 97% or 100% of the cells express cellular retinaldehyde binding protein (CRALBP), as measured by immunostaining. For example, between 80-100% of the cells express CRALBP.

According to another embodiment, at least 80%, 85%, 87%, 89%, 90%, 95%, 97% or 100% of the cells express cellular Melanocytes Lineage-Specific Antigen GP100 (PMEL17), as measured by immunostaining. For example, between about 80-100% of the cells express PMEL17.

The RPE cells may co-express markers indicative of terminal differentiation, e.g. bestrophin 1, CRALBP and/or RPE65. According to one embodiment, at least 95%, at least 96%, at least 97%, at least 98%, at least 99%, at least 100%, or even between about 50% to 100% of the cells of the RPE cell populations obtained co-express both premelanosome protein (PMEL17) and cellular retinaldehyde binding protein (CRALBP).

According to a particular embodiment, the cells coexpress PMEL17 (SwissProt No. P40967) and at least one polypeptide selected from the group consisting of cellular retinaldehyde binding protein (CRALBP; SwissProt No. P12271), lecithin retinol acyltransferase (LRAT; SwissProt No. 095327) and sex determining region Y-box 9 (SOX 9; P48436).

According to a particular embodiment, at least 80% of the cells of the population express detectable levels of PMEL17 and one of the above mentioned polypeptides (e.g. CRALBP), more preferably at least 85% of the cells of the population express detectable levels of PMEL17 and one of the above mentioned polypeptides (e.g. CRALBP), more preferably at least 90% of the cells of the population express detectable levels of PMEL17 and one of the above mentioned polypeptides (e.g. CRALBP), more preferably at least 95% of the cells of the population express detectable levels of PMEL17 and one of the above mentioned polypeptides (e.g. CRALBP), more preferably 100% of the cells of the population express detectable levels of PMEL17 and one of the above mentioned polypeptides (e.g. CRALBP as assayed by a method known to those of skill in the art (e.g. FACS).

According to another embodiment, the level of CRALBP and one of the above-mentioned polypeptides (e.g. PMEL17) coexpression (e.g. as measured by the mean fluorescent intensity) is increased by at least two fold, more preferably at least 3 fold, more preferably at least 4 fold and even more preferably by at least 5 fold, at least 10 fold, at least 20 fold, at least 30 fold, at least 40 fold, at least 50 fold as compared to non-differentiated ESCs.

In one embodiment, the RPE are terminally differentiated and do not generally express Pax6. In another embodiment, the RPE cells are terminally differentiated and generally express Pax6.

The RPE cells described herein may also act as functional RPE cells after transplantation wherein the RPE cells may form a monolayer between the neurosensory retina and the choroid in the patient receiving the transplanted cells. The RPE cells may also supply nutrients to adjacent photoreceptors and dispose of shed photoreceptor outer segments by phagocytosis.

According to one embodiment, the trans-epithelial electrical resistance of the cells in a monolayer is greater than 100 ohms.

Preferably, the trans-epithelial electrical resistance of the cells is greater than 150, 200, 250, 300, 300, 400, 500, 600, 700, 800 or even greater than 900 ohms. The resistance may be any value or subrange within the recited ranges, including endpoints.

Devices for measuring trans-epithelial electrical resistance (TEER) are known in the art and include for example EVOM2 Epithelial Voltohmmeter, (World Precision Instruments).

Following the expansion phase, cell populations comprising RPE cells are obtained whereby at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% thereof are CRALBP+PMEL1 7+.

It would be well appreciated by those versed in the art that the derivation of RPE cells is of great benefit. They may be used as an in vitro model for the development of new drugs to promote their survival, regeneration and function. RPE cells may serve for high throughput screening for compounds that have a toxic or regenerative effect on RPE cells. They may be used to uncover mechanisms, new genes, soluble or membrane-bound factors that are important for the development, differentiation, maintenance, survival and function of photoreceptor cells.

The RPE cells described herein may also serve as an unlimited source of RPE cells for transplantation, replenishment and support of malfunctioning or degenerated RPE cells in retinal degenerations and other degenerative disorders. Furthermore, genetically modified RPE cells may serve as a vector to carry and express genes in the eye and retina after transplantation.

In certain embodiments, RPE cell compositions may be produced according to following methods: (1) culturing hESCs on hUCFs in CW plates for 2 weeks in NUT+ with human serum albumin (HSA), (2) mechanical passaging to expand the hESCs on hUCFs in CW plates for between four to five weeks (or until desired amount of cells) in NUT+ with HSA, (3) continue to expand hESC colonies (using for example, collagenase) on hUCFs in 6 cm plates for an additional week in NUT+ with HSA, (4) prepare spheroid bodies (SB) by transferring colonies from about five 6 cm plates into 1 HydroCell for about one week in NUT− with nicotinamide (NIC), (5) flattening of SBs on Lam511 may be carried out by transferring the SBs to 2-3 wells of a 6-well plate for about one week in NUT− with NIC, (6) culture adherent cells on Lam511 in NUT− with NIC and Activin for about one to two weeks and replace media with NUT− with NIC and culture for between one and three weeks, (7) enrich for pigmented cells using enzymes, such as TrypLE Select for example, (8) expand RPE cells on gelatin in flasks for between about two to nine weeks (replacing media) in 20% human serum and NUT−, and (9) harvest RPE cells.

Harvesting of the expanded population of RPE cells may be effected using methods known in the art (e.g. using an enzyme such as trypsin, or chemically using EDTA, etc). In some embodiments, the RPE cells may be washed using an appropriate solution, such as PBS or BSS plus. In other embodiments, the RPE cells may be filtered prior to formulation of the RPE cell compositions for cryopreservation and administration to a subject directly after thawing. In some embodiments, the percent viability of post-filtered cells is at least about 75%, 80%, 85%, 90%, 91%, 92%, 93%, 94%, 95%, 96%, 97%, 98%, 99%, or 100%. In some embodiments, the percent viability of post-filtered cells stored in a neutralization solution for between about 0 to about 8 hours is at least about 75%, 80%, 85%, 90%, 91%, 92%, 93%, 94%, 95%, 96%, 97%, 98%, 99%, or 100%.

In further embodiments, the percent viability of post-filtered cells stored in a neutralization medium for between about 0 to about 8 hours followed by storage in cryopreservation medium for between about 0 to about 8 hours is at least about 75%, 80%, 85%, 90%, 91%, 92%, 93%, 94%, 95%, 96%, 97%, 98%, 99%, or 100%. In other embodiments, the percent recovery of post-filtered cells stored in a neutralization medium for between about 0 to about 8 hours followed by storage in cryopreservation medium for between about 0 to about 8 hours is at least about 70%, 75%, 80%, 85%, 90%, 91%, 92%, 93%, 94%, 95%, 96%, 97%, 98%, 99%, or 100%.

In yet other embodiments, the percent viability of post-filtered cells stored in a neutralization medium for between about 0 to about 8 hours followed by storage in cryopreservation medium for between about 0 to about 8 hours, post-thawing of the cryopreserved composition, is at least about 75%, 80%, 85%, 90%, 91%, 92%, 93%, 94%, 95%, 96%, 97%, 98%, 99%, or 100%. In still other embodiments, the percent recovery of post-filtered cells stored in a neutralization medium for between about 0 to about 8 hours followed by storage in cryopreservation medium for between about 0 to about 8 hours, post-thawing of the cryopreserved composition, is at least about, 70%, 75%, 80%, 85%, 90%, 91%, 92%, 93%, 94%, 95%, 96%, 97%, 98%, 99%, or 100%.

In some embodiments, post-filtered RPE cells stored in a neutralization medium for between about 0 to about 8 hours followed by storage in cryopreservation medium for between about 0 to about 8 hours, post-thawing of the cryopreserved composition are capable of secreting PEDF at between about 1,500 ng/ml/day to about 4,500 ng/ml/day, about 2,000 ng/ml/day to about 3,000 ng/ml/day. The concentration may be any value or subrange within the recited ranges, including endpoints. In other embodiments, post-filtered RPE cells stored in a neutralization medium for between about 0 to about 8 hours followed by storage in cryopreservation medium for between about 0 to about 8 hours, post-thawing of the cryopreserved composition are capable of being expanded to at least between about 1.2×10⁶ and 5×10⁶, or about 2.5×10⁶ to about 4×10⁶ cells in 14 days.

In some embodiments, the percent viability of post-filtered RPE cells stored in a neutralization medium for between about 0 to about 8 hours at room temperature is at least about, 70%, 75%, 80%, 85%, 90%, 91%, 92%, 93%, 94%, 95%, 96%, 97%, 98%, 99%, or 100%. In some embodiments, the percent viability of post-filtered RPE cells stored in a cryopreservation medium for between about 0 to about 8 hours at room temperature is at least about, 70%, 75%, 80%, 85%, 90%, 91%, 92%, 93%, 94%, 95%, 96%, 97%, 98%, 99%, or 100%. In further embodiments, the percent viability of post-filtered cells stored in a neutralization solution at room temperature for between about 0 to about 8 hours followed by storage in cryopreservation medium for between about 0 to about 8 hours at room temperature is at least about 75%, 80%, 85%, 90%, 91%, 92%, 93%, 94%, 95%, 96%, 97%, 98%, 99%, or 100%. In still further embodiments, the percent recovery of post-filtered cells stored in a neutralization solution at room temperature for between about 0 to about 8 hours followed by storage in cryopreservation medium for between about 0 to about 8 hours at room temperature is at least about 75%, 80%, 85%, 90%, 91%, 92%, 93%, 94%, 95%, 96%, 97%, 98%, 99%, 100%, 105%, 110%, 115%, 120%, 125%, 130%, 140%, 150%.

Following harvesting, the expanded population of RPE cells can be formulated at a specific therapeutic dose (e.g., number of cells) and cryopreserved for shipping to the clinic. The ready to administer (RTA) RPE cell therapy composition can then be administered directly after thawing without further processing. Examples of media suitable for cryopreservation include but are not limited to 90% Human Serum/10% DMSO, Media 3 10% (CS10), Media 2 5% (CS5) and Media 1 2% (CS2), Stem Cell Banker, PRIME XVº FREEZIS, HYPOTHERMASOL®, Trehalose, etc.

RPE cells formulated in cryopreservation media appropriate for post thaw ready to administer (RTA) applications may comprise RPE cells suspended in adenosine, dextran 40, lactobionic acid, HEPES (N-(2-Hydroxyethyl) piperazine N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water. An example of this cryopreservation medium is available commercially under the tradename, CRYOSTOR® and is manufactured by BioLife Solutions, Inc.

In further embodiments, the cryopreservation medium includes: a purine nucleoside (e.g., adenosine), a branched glucan (e.g., dextran 40), a zwitterionic organic chemical buffering agent (e.g., HEPES (N-(2-Hydroxyethyl) piperazine EN′-(2E ethanesulfonic acid))), and a cell tolerable polar aprotic solvent (e.g., dimethyl sulfoxide (DMSO). In still further embodiments, one or more of the purine nucleoside, branched glucan, buffering agent, and the polar aprotic solvent are generally recognized as safe by the US FDA.

In some embodiments, the cryopreservation media further includes one or more of: a sugar acid (e.g., lactobionic acid), one or more of a base (e.g., sodium hydroxide, potassium hydroxide), an antioxidant (e.g., L-glutathione), one or more halide salt (e.g., potassium chloride, sodium chloride, magnesium chloride), a basic salt (e.g., potassium bicarbonate), phosphate salt (e.g., potassium phosphate, sodium phosphate, potassium phosphate), one or more sugars (e.g., dextrose, sucrose), sugar alcohol, (e.g., mannitol), and water.

In other embodiments, one or more of the sugar acid, base, halide salt, basic salt, antioxidant, phosphate salt, sugars, sugar alcohols are generally recognized as safe by the US FDA.

DMSO can be used as a cryoprotective agent to prevent the formation of ice crystals, which can kill cells during the cryopreservation process. In some embodiments, the cryopreservable RPE cell therapy composition comprises between about 0.1% and about 2% DMSO (v/v). In some embodiments, the RTA RPE cell therapy composition comprises between about 1% and about 20% DMSO. In some embodiments, the RTA RPE cell therapy composition comprises about 2% DMSO. In some embodiments, the RTA RPE cell therapy composition comprises about 5% DMSO.

In some embodiments, RPE cell therapies formulated in cryopreservation media appropriate for post thaw ready to administer applications may comprise RPE cells suspended in cryopreservation media that does not contain DMSO. For example, RTA RPE cell therapy compositions may comprise RPE cells suspended in Trolox, Na+, K+, Ca2+, Mg2+, cl−, H2P04—HEPES, lactobionate, sucrose, mannitol, glucose, dextran-40, adenosine, glutathione without DMSO (dimethyl sulfoxide, (CH3)2SO) or any other dipolar aprotic solvents. An example of this cryopreservation media is available commercially under the tradename, HYPOTHERMOSOL® or HYPOTHERMOSOL®-FRS and is also manufactured by BioLife Solutions, Inc. In other embodiments, RPE cell compositions formulated in cryopreservation media appropriate for post thaw ready to administer applications may comprise RPE cells suspended in Trehalose.

RTA RPE cell therapies formulated according to the present disclosure do not require the use of GMP facilities for preparation of the final dose formulation prior to injection into a subject's eye. The RTA RPE cell therapy formulations described herein may be cryopreserved in a non-toxic cryosolution that comprises the final dose formulation which can be shipped directly to the clinical site. When needed, the formulation can be thawed and administered into the subject's eye without having to perform any intermediate preparation steps.

In some embodiments, the RPE cell composition may be cryopreserved and stored at a temperature of between about −4° C. to about −200° C. In some embodiments, the RPE cell composition may be cryopreserved and stored at a temperature of between about −20° C. to about −200° C. In some embodiments, the RPE cell composition may be cryopreserved and stored at a temperature of between about −70° C. to about −196° C. In some embodiments, the temperature adequate for cryopreservation or a cryopreservation temperature, comprises a temperature of between about −4° C. to about −200° C., or a temperature of between about −20° C. to about −200° C., −70° C. to about −196° C.

In some embodiments, the RTA RPE cell therapy composition may be stored frozen for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In other embodiments, the RPE cells may be stored frozen for between about 1.5 to 48 months. In other embodiments, the RTA RPE cell therapy composition may be stored frozen for between about 1 to about 48 months without a decrease in percent viability or cell recovery. In some embodiments, the RTA RPE cell therapy composition may be stored for at least about 38 hours at 2-8° C., while maintaining stability.

In some embodiments, the RTA RPE cell therapy composition may be shipped frozen over 8,000 miles without a decrease in percent viability, percent cell recovery, or potency.

RPE cells can be produced, for example, according to the methods of Idelson M, Alper R, Obolensky A et al. (Directed differentiation of human embryonic stem cells into functional retinal pigment epithelium cells. Cell Stem Cell 2009; 5:396-408) or according to Parul Choudhary et al, (“Directing Differentiation of Pluripotent Stem Cells Toward Retinal Pigment Epithelium Lineage”, Stem Cells Translational Medicine, 2016), or WO 2008129554, all of which are incorporated herein by reference in their entirety.

The RTA RPE cell therapy composition may optionally comprise additional factors that support RPE engraftment, integration, survival, potency, etc. In some embodiments, the RTA RPE cell therapy composition comprises activators of function of the RPE cell preparations described herein. In some embodiments, the RTA RPE cell therapy composition comprises nicotinamide. In some embodiments, the RTA RPE cell therapy composition comprises nicotinamide at a concentration of between about 0.01-100 mM, 0.1-100 mM, 0.1-50 mM, 5¬50 mM, 5-20 mM, e.g. 10 mM. In other embodiments, the RTA RPE cell therapy composition comprises retinoic acid. In some embodiments, the RTA RPE cell therapy composition comprises retinoic acid at a concentration of between about 0.01-100 mM, 0.1-100 mM, 0.1-50 mM, 5-50 mM, 5-20 mM, e.g. 10 mM. The concentration may be any value or subrange within the recited ranges, including endpoints.

In some embodiments, the RTA RPE cell therapy composition may be formulated to include activators of various integrins that have been shown to increase the adherence of the RPE cell preparations, such as those described herein, to the Brunch's membrane. For example, in some embodiments, the RTA RPE cell therapy composition comprises extracellular manganese (Mn2+) at a concentration of between about 5 μM and 1,000 μM. In other embodiments, the RTA RPE cell therapy composition comprises the conformation-specific monoclonal antibody, TS2/16.

In other embodiments, the RTA RPE cell therapy composition may also be formulated to include activators of RPE cell immune regulatory activity.

In some embodiments, the RTA RPE cell therapy composition may include a ROCK inhibitor.

In some embodiments, the RTA RPE cell therapy composition may be formulated in a medium comprising components that decrease the molecular cell stress during freezing and thawing processes by scavenging of free radicals, pH buffering, oncotic/osmotic support and maintenance of the ionic concentration balance.

In some embodiments, RPE cell therapies formulated in cryopreservation media appropriate for post thaw ready to administer applications may comprise one or more immunosuppressive compounds. In certain embodiments, RPE cell therapies formulated in cryopreservation media appropriate for post thaw ready to administer applications may comprise one or more immunosuppressive compounds that are formulated for slow release of the one or more immunosuppressive compounds. Immunosuppressive compounds for use with the formulations described herein may belong to the following classes of immunosuppressive drugs: Glucocorticoids, Cytostatics (e.g. alkylating agent or antimetabolite), antibodies (polyclonal or monoclonal), drugs acting on immunophilins (e.g. cyclosporin, Tacrolimus or Sirolimus). Additional drugs include interferons, opioids, TNF binding proteins, mycophenolate and small biological agents. Examples of immunosuppressive drugs include: mesenchymal stem cells, anti-lymphocyte globulin (ALG) polyclonal antibody, anti-thymocyte globulin (ATG) polyclonal antibody, azathioprine, BAS 1L1 X 1MAB® (anti-IL-2Ra receptor antibody), cyclosporin (cyclosporin A), DACLIZUMAB® (anti-IL-2Ra receptor antibody), everolimus, mycophenolic acid, RITUXUMAB® (anti-CD20 antibody), sirolimus, tacrolimus, Tacrolimus and or Mycophenolate mofetil.

Further methods for generating RPE cells as envisioned within the present disclosure are described in PCT/US2018/023030 (WO 2018/170494), the contents of which are incorporated by reference herein, in their entirety.

Further methods for generating “thaw and inject” formulations as envisioned within the present disclosure are described in PCT/IB2018/001579 (WO 2019/130061), the contents of which are incorporated by reference herein, in their entirety.

In certain embodiments, the RPE cell therapy may be formulated at a cell concentration of between about 100,000 cells/ml to about 1,000,000 cells/ml. In certain embodiments, the RPE cell therapy may be formulated at a cell concentration of about 1,000,000 cells/ml, about 2,000,000 cells/ml, about 3,000,000 cells/ml, about 4,000,000 cells/ml, about 5,000,000 cells/ml, 6,000,000 cells/ml, 7,000,000 cells/ml, 8,000,000 cells/ml, about 9,000,000 cells/ml, about 10,000,000 cells/ml, about 11,000,000 cells/ml, about 12,000,000 cells/ml, 13,000,000 cells/ml, 14,000,000 cells/ml, 15,000,000 cells/ml, 16,000,000 cells/ml, about 17,000,000 cells/ml, about 18,000,000 cells/ml, about 19,000,000 cells/ml, or about 20,000,000 cells/ml. The cell concentration may be any value or subrange within the recited ranges, including endpoints.

In some embodiments, the RPE cells are administered in a therapeutically or pharmaceutically acceptable carrier or biocompatible medium. In some embodiments, the volume of the RPE formulation administered to the subject is between about 10 μl to about 50 μl, about 20 μl to about 70 μl, about 20 μl to about 100 μl, about 25 μl to about 100 μl, about 100 μl to about 150 μl, or about 10 μl to about 200 μl. In certain embodiments, two or more doses of between 10 μl and 200 μl of the RPE formulation can be administered. In certain embodiments, the volume of RPE formulation is administered to the subretinal space of a subject's eye. In certain embodiments, the subretinal delivery method can be transvitreal or suprachoroidal. In some embodiments, for some subjects, the incidents of ERM may be reduced using a transvitreal or suprachoroidal subretinal delivery method. In some embodiments, the volume of RPE formulation can be injected into the subject's eye.

In some embodiments, the RPE cells of the cell therapeutic agent are human RPE cells.

In some embodiments, the RPE cells are OpRegen® cells. OpRegen is a RPE cell line derived from a human embryonic (hESC) cell line under low oxygen (5%) culture supplemented with high concentration of Activin A, a transforming growth factor beta (TGF-b) family and nicotinamide before switching to normal oxygen (20%) culture to enrich RPE population. Activin A improves RPE cell survival on rigid or stiff but not soft substrates. As such, OpRegen has gained additional biological competence as compared to native RPE cells enhancing survival in a harsh microenvironment such as in the GA setting, where Bruch's membrane degenerates and becomes rigid or thickening. Among the over 120+ identified proteins secreted by OpRegen cell, pigment epithelial derived factor (PEDF), platelet derived growth factor (PDGF), vascular endothelial growth factor (VEGF), bestrophine, angiogenin, CRLABP, TIMP-2, TIMP-1, IL-6, PMEL-1 (melonosome), integrin, TNF-α, and complement protection proteins are topped as high level secretive proteins. Its potency has been tested by basal PEDFNEGF ratio and apical VEGF/PEDF ratio at day 21, of which both were >1. Of note, high oxygen level increases PEDF secretion. OpRegen at suspension formula can still generate PEDF at 2-8° C. for 24 hours, which indicates its robustness.

OpRegen secretes very high levels of PEDF at 2000-4000 ng/ml/day, which can explain its high therapeutic potency as PEDF has anti-oxidative roles in RPE to the BRB which is of interest to AMD indication. PEDF is a 50 kDa protein secreted by RPE and Muller glia in vivo; it also demonstrates neuroprotective function for photoreceptors, possibly through restoring mitochondrial dynamics perturbed by aging and oxidative stress. PEDF could prevent H₂O₂ induced RPE permeability changes and preserve the barrier function of RPE against oxidative stress. PEDF is also endogenous anti-inflammatory factor through its interaction with master factor NF-KappaB. PEDF binds to extracellular matrix (collagen and proteoglycans) and has a role in anti-fibrosis in diabetic retinopathy and wet AMD through the inhibition of TGF-beta. In part, PEDF secretion supports findings in OpRegen-treated subjects, as evidenced by fluorescein angiography (FA) improvement in those with/without drusen, and OCT imaging with possible signs of ECM remodeling or scar attenuation within the GA lesion seen as early as 2-4 weeks post-transplant.

The RPE cells suitable for use within the scope of the present disclosure is not limited to the RPE cells described herein. Any commercially, or otherwise available RPE cells may be used.

In some embodiments, the cell therapeutic agent described herein is capable of restoring retinal structure of retinal disease.

Restoring the anatomy of a retina of a patient may be used interchangeably with ‘restoration’ and ‘restoring’ and means the restoring or recovery of the normal architecture of a patient as compared to age-matched, sex-matched control, a baseline, or a fellow eye; restoration of areas of normal anatomical structure as determined by changes in the ellipsoid zone (EZ) in affected areas, RPE engraftment as evidenced by OCT, and improved retinal thickness; restoring or inducing regeneration of retinal pigment epithelium (RPE); restoration of areas of normal anatomical structure as determined by changes in the ellipsoid zone (EZ) in affected areas, RPE engraftment as evidenced by OCT, and improved retinal thickness; restoration of vision; decreases an atrophy area in an atrophic retina; restoring one or more retinal layers of the retina; restoring photoreceptors of the retina; restoring the outer nuclear layer (ONL) of a retina; restoring the ellipsoid zone (EZ) of a retina; restoring the fovea of a retina; restoring the blood-retinal barrier (BRB) of a retina; and restoring the extracellular matrix (ECM) of a retina.

Restoring or recovering the functionality of a retina of a patient means that the retinal layers are restored to their normal structure and that the RPE cell performing activities, such as light absorption, epithelial transport, phagocytosis of photoreceptor outer segment (POS) membranes, and secretion of factors such as PEDF and photoreceptor are functionally active and able to carry out phototransduction, thereby enabling functional vision.

“Recovery” and “recover” and “recovers” and “recovering” may be used interchangeably to mean recovery of an ellipsoid zone; recovery by restoration of normal architecture; as compared to age-matched, sex-matched control, a baseline or a fellow eye; the subjective assessment that one or more of the following are becoming more organized, including the external limiting membrane, myoid zone (inner segments of photoreceptors), ellipsoid zone (IS/OS Junction), outer segments of the photoreceptors, loss of drusen, and disappearance of reticular pseudo-drusen; the subjective assessment that one or more of the basic foundational layers of the retina are becoming more organized including but not limited to one or more of the external limiting membrane, myoid zone (inner segments of photoreceptors), ellipsoid zone (IS/OS Junction), and outer segments of the photoreceptors; demonstrating that sites of the retina near or at the site of administration of the RPE cells comprises an improved microperimetry assessment compared to a baseline microperimetry assessment; recovery of an ellipsoid zone comprising improvement in one or more of, EZ-RPE thickness, area, or volume measurements; EZ-RPE central foveal mean thickness improvement; EZ-RPE central foveal thickness improvement; EZ-RPE central subfield volume improvement; recovery of pigment epithelium and retinal thickness; organization of the basic foundational layers of the retina; and organization of 2-6 of the 12-14 layers of the retina.

Treatment and Dosage

The number of viable cells that may be administered to the subject are typically between at least about 50,000 and about 5×10⁶ per dose. In some embodiments, the cell therapeutic agent comprises at least about 50,000 viable cells. In some embodiments, the cell therapeutic agent comprises at least about 100,000 viable cells. In some embodiments, the cell therapeutic agent comprises at least about 150,000 viable cells. In some embodiments, the cell therapeutic agent comprises at least about 200,000 viable cells. In some embodiments, the cell therapeutic agent comprises at least about 250,000 viable cells. In some embodiments, the cell therapeutic agent comprises at least about 300,000 viable cells. In some embodiments, the cell therapeutic agent comprises at least about 350,000 viable cells. In some embodiments, the cell therapeutic agent comprises at least about 400,000 viable cells. In some embodiments, the cell therapeutic agent comprises at least about 450,000 viable cells. In some embodiments, the cell therapeutic agent comprises at least about 500,000 viable cells. In some embodiments, the cell therapeutic agent comprises at least about 600,000, at least about 700,000, at least about 800,000, at least about 900,000, at least about 1,000,000, at least about 2,000,000, at least about 3,000,000, at least about 4,000,000, at least about 5,000,000 at least about 6,000,000, at least about 7,000,000, at least about 8,000,000, at least about 9,000,000, at least about 10,000,000, at least about 11,000,000, or at least about 12,000,000 viable cells. In some embodiments, the cell therapeutic agent comprises between 50,000 and 100,000 viable cells. In some embodiments, the cell therapeutic agent comprises between 100,000 and 200,000 viable cells. In some embodiments, the cell therapeutic agent comprises between 200,000 and 300,000 viable cells. In some embodiments, the cell therapeutic agent comprises between 300,000 and 400,000 viable cells. In some embodiments, the cell therapeutic agent comprises between 400,000 and 500,000 viable cells. In some embodiments, the cell therapeutic agent comprises between 500,000 and 1,000,000 viable cells. In some embodiments, the cell therapeutic agent comprises between 1,000,000 and 2,000,000 viable cells. In some embodiments, the cell therapeutic agent comprises between 2,000,000 and 3,000,000 viable cells. In some embodiments, the cell therapeutic agent comprises between 3,000,000 and 4,000,000 viable cells. In some embodiments, the cell therapeutic agent comprises between 4,000,000 and 5,000,000 viable cells. In some embodiments, the cell therapeutic agent comprises between 5,000,000 and 6,000,000 viable cells. In some embodiments, the cell therapeutic agent comprises between 6,000,000 and 7,000,000 viable cells. In some embodiments, the cell therapeutic agent comprises between 7,000,000 and 8,000,000 viable cells. In some embodiments, the cell therapeutic agent comprises between 8,000,000 and 9,000,000 viable cells. In some embodiments, the cell therapeutic agent comprises between 9,000,000 and 10,000,000 viable cells. In some embodiments, the cell therapeutic agent comprises between 10,000,000 and 11,000,000 viable cells. In some embodiments, the cell therapeutic agent comprises between 11,000,000 and 12,000,000 viable cells. In specific embodiments, the cell therapeutic agent is administered at a dose of 50,000 to 1,000,000 cells. In specific embodiments, the cell therapeutic agent is administered at a dose of 100,000 to 750,000 cells. In specific embodiments, the cell therapeutic agent is administered at a dose of 200,000 to 500,000 cells. Each of the values or ranges recited herein may include any value or subrange therebetween, including endpoints.

In some embodiments, the volume of the RTA RPE formulation administered to the subject is between about 50 μl to about 100 μl, about 25 μl to about 100 μl, about 100 μl to about 150 μl, or about 10 μl to about 200 μl. In certain embodiments, two doses of between 10 μl and 200 μl of the RTA RPE formulation can be administered. Each of the values or ranges recited herein may include any value or subrange therebetween, including endpoints.

In certain embodiments, the volume of RTA RPE formulation is administered to the subretinal space of a subject's eye. In certain embodiments, the subretinal delivery method can be transvitreal or suprachoroidal. In some embodiments, the volume of RTA RPE formulation can be injected into the subject's eye.

In certain embodiments, the RTA RPE therapeutic cell compositions may be formulated at a cell concentration of between about 100,000 cells/ml to about 1,000,000 cells/ml. In certain embodiments, the RTA RPE cell therapy may be formulated at a cell concentration of about 1,000,000 cells/ml, about 2,000,000 cells/ml, about 3,000,000 cells/ml, about 4,000,000 cells/ml, about 5,000,000 cells/ml, 6,000,000 cells/ml, 7,000,000 cells/ml, 8,000,000 cells/ml, about 9,000,000 cells/ml, about 10,000,000 cells/ml, about 11,000,000 cells/ml, about 12,000,000 cells/ml, 13,000,000 cells/ml, 14,000,000 cells/ml, 15,000,000 cells/ml, 16,000,000 cells/ml, about 17,000,000 cells/ml, about 18,000,000 cells/ml, about 19,000,000 cells/ml, or about 20,000,000 cells/ml. Each of the values or ranges recited herein may include any value or subrange therebetween, including endpoints.

In embodiments, the method includes administering RPE cells to a subject's eye. In embodiments, the method includes administering RPE cells in the subretinal space of the subject's eye. In embodiments, the method includes administering RPE cells into the vitreal space, inner or outer retina, the retinal periphery, or within the choroids of the subject's eye. In embodiments, the method includes administering RPE cells over a GA lesion. In embodiments, the method includes targeting the GA in a subject's eye. In embodiments, the method includes administering RPE cells by lifting the GA. In embodiments, the method includes administering RPC cells over surrounding healthy tissue near a GA lesion. In embodiments, the RPE cells are administered as a monolayer. In some embodiments, the cell composition is injected.

The RPE cells generated as described herein may be transplanted to various target sites within a subject's eye or other locations (for example in the brain). In accordance with one embodiment, the transplantation of the RPE cells is to the subretinal space of the eye, which is the normal anatomical location of the RPE (between the photoreceptor outer segments and the choroid). In addition, dependent upon migratory ability and/or positive paracrine effects of the cells, transplantation into additional ocular compartments can be considered including but not limited to the vitreal space, inner or outer retina, the retinal periphery and within the choroids.

The transplantation may be performed by various techniques known in the art. Methods for performing RPE transplants are described in, for example, U.S. Pat. Nos. 5,962,027, 6,045,791, and 5,941,250 and in Eye Graefes Arch Clin Exp Opthalmol March 1997; 235(3): 149-58; Biochem Biophys Res Commun Feb. 24, 2000; 268(3): 842-6; Opthalmic Surg February 1991; 22(2): 102-8. Methods for performing corneal transplants are described in, for example, U.S. Pat. No. 5,755,785, and in Eye 1995; 9 (Pt 6 Su):6-12; Curr Opin Opthalmol August 1992; 3 (4): 473-81; Ophthalmic Surg Lasers April 1998; 29 (4): 305-8; Ophthalmology April 2000; 107 (4): 719-24; and Jpn J Ophthalmol November-December 1999; 43(6): 502-8. If mainly paracrine effects are to be utilized, cells may also be delivered and maintained in the eye encapsulated within a semi-permeable container or biodegradable extracellular matrix, which will also decrease exposure of the cells to the host immune system (Neurotech USA CNTF delivery system; PNAS Mar. 7, 2006 vol. 103(10) 3896-3901).

In some embodiments, the cell therapeutic agent is implanted adjacent to the atrophic retina.

In embodiments, the cell therapeutic agent is administered adjacent to the GA. In embodiments, the cell therapeutic agent is administered to the GA. In embodiments, the cell therapeutic agent covers at least about 20% of the GA after administration. In embodiments, the cell therapeutic agent covers at least about 30% of the GA after administration. In embodiments, the cell therapeutic agent covers at least about 40% of the GA after administration. In embodiments, the cell therapeutic agent covers at least about 50% of the GA after administration. In embodiments, the cell therapeutic agent covers at least about 60% of the GA after administration. In embodiments, the cell therapeutic agent covers at least about 70% of the GA after administration. In embodiments, the cell therapeutic agent covers at least about 75% of the GA after administration. In embodiments, the cell therapeutic agent covers at least about 80% of the GA after administration. In embodiments, the cell therapeutic agent covers at least about 85% of the GA after administration. In embodiments, the cell therapeutic agent covers at least about 90% of the GA after administration. In embodiments, the cell therapeutic agent covers at least about 95% of the GA after administration. In embodiments, the cell therapeutic agent covers at least about 96% of the GA after administration. In embodiments, the cell therapeutic agent covers at least about 97% of the GA after administration. In embodiments, the cell therapeutic agent covers at least about 98% of the GA after administration. In embodiments, the cell therapeutic agent covers at least about 99% of the GA after administration. In embodiments, the cell therapeutic agent covers about 100% of the GA after administration.

In accordance with one embodiment, transplantation is performed via pars plane vitrectomy surgery followed by delivery of the cells through a small retinal opening into the sub-retinal space or by direct injection.

In certain embodiments, administration may comprise a vitrectomy followed by delivery of the RTA therapeutic cell composition into the subretinal space in the macular area via a cannula through a small retinotomy. A total volume of 50-100 μL cell suspension, depending on the cell dose, can be implanted in areas at potential risk for GA expansion.

In some embodiments, a single surgical procedure is performed in which the RTA therapeutic cell composition is delivered through a small retinotomy, following vitrectomy, into a subretinal space created in the macular area, along the border between areas of GA, if present, and the better preserved extra-foveal retina and RPE layer. After the placement of a lid speculum, a standard 3-port vitrectomy can be performed. This may include the placement of a 23G or 25G infusion cannula and two 23G or 25/23G ports (trocars). A core vitrectomy can then be performed with 23G or 25G instruments, followed by detachment of the posterior vitreous face. The RTA therapeutic cell composition may be injected into the subretinal space at a predetermined site within the posterior pole, preferably penetrating the retina in an area that is still relatively preserved close to the border of GA, if present.

In some embodiments, the cell composition is administered by a suprachoroidal injection.

The RPE cells may be transplanted in various forms. For example, the RPE cells may be introduced into the target site in the form of single cell suspension, with matrix or adhered onto a matrix or a membrane, extracellular matrix or substrate such as a biodegradable polymer or a combination. The RPE cells may also be printed onto a matrix or scaffold. The RPE cells may also be transplanted together (co-transplantation) with other retinal cells, such as with photoreceptors. The effectiveness of treatment may be assessed by different measures of visual and ocular function and structure, including, among others, best corrected visual acuity (BCVA), retinal sensitivity to light as measured by perimetry or microperimetry in the dark and light-adapted states, full-field, multi-focal, focal or pattern electroretinography 5 ERG), contrast sensitivity, reading speed, color vision, clinical biomicroscopic examination, fundus photography, optical coherence tomography (OCT), fundus auto-fluorescence (FAF), infrared and multicolor imaging, fluorescein or ICG angiography, adoptive optics and additional means used to evaluate visual function and ocular structure.

The subject may be administered corticosteroids prior to or concurrently with the administration of the RPE cells, such as prednisolone or methylprednisolone, Predforte. According to another embodiment, the subject is not administered corticosteroids prior to or concurrently with the administration of the RPE cells, such as prednisolone or methylprednisolone, Predforte.

Immunosuppressive drugs may be administered to the subject prior to, concurrently with and/or following treatment. The immunosuppressive drug may belong to the following classes: Glucocorticoids, Cytostatics (e.g. alkylating agent or antimetabolite), antibodies (polyclonal or monoclonal), drugs acting on immunophilins (e.g. cyclosporin, Tacrolimus or Sirolimus). Additional drugs include interferons, opioids, TNF binding proteins, mycophenolate and small biological agents. Examples of immunosuppressive drugs include: mesenchymal stem cells, anti-lymphocyte globulin (ALG) polyclonal antibody, anti-thymocyte globulin (ATG) polyclonal antibody, azathioprine, BAS 1L1 X 1MABO (anti-IL-2Ra receptor antibody), cyclosporin (cyclosporin A), DACLIZUMAB® (anti-IL-2Ra receptor antibody), everolimus, mycophenolic acid, RITUX 1MABO (anti-CD20 antibody), sirolimus, tacrolimus, Tacrolimus and or Mycophenolate mofetil.

Immunosuppressive drugs may be administered to the subject, for example, topically, intraocularly, intraretinally, or systemically. Immunosuppressive drugs may be administered in one or more of those methods at the same time or the delivery methods may be used in a staggered method.

Alternatively, the RTA RPE cell therapy composition may be administered without the use of immunosuppressive drugs.

Antibiotics may be administered to the subject prior to, concurrently with and/or following treatment. Examples of antibiotics include Oflox, Gentamicin, Chloramphenicol, Tobrex, Vigamox or any other topical antibiotic preparation authorized for ocular use.

In some embodiments, the cell composition does not cause inflammation after it is administered. In some embodiments, the inflammation may be characterized by the presence of cells associated with inflammation.

In some embodiments, the restoring leads to a decrease in atrophy area. At specified times after treatment, fundus autofluorescence (FAF) can then be used to detect any hyperfluorescence, particularly around the rim of the lesion and the size of the area of atrophy can be measured. In addition to the decrease in overall size of the lesion, a decrease in the size or disappearance of the hyperfluorescent rim around the periphery of the lesion can be used to indicate that the treatment is slowing down or arresting disease progression. The difference in hyperfluorescence between the treated half of the lesion and the nontreated half of the lesion can be measured and used to determine the efficacy of the treatment. As such, the same eye may be used as a treatment subject and control subject.

In some embodiments, the restoring leads to a decrease in atrophy area. As used herein, the terms “decrease,” “reduce,” “reduction,” “minimal,” “low,” or “lower” refer to decreases below basal levels, e.g., as compared to a control. The terms “increase,” high,” “higher,” “maximal,” “elevate,” or “elevation” refer to increases above basal levels, e.g., as compared to a control. Increases, elevations, decreases, or reductions can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a control or standard level. Each of the values or ranges recited herein may include any value or subrange therebetween, including endpoints.

In certain embodiments, the treatment leads to restoration of retinal layers. In another embodiment, treatment effect assessment using the two-dimensional imaging of fundus autofluorescence is augmented using optical coherence tomography (OCT). OCT can be used to generate three-dimensional high-resolution images and can provide important cross-sectional information for the structural assessment of retinal layers, particularly in subjects being treated for retinal diseases. Using OCT, profile images of the layers of the retina can be obtained before and after treatment for a retinal disorder has been administered. In healthy eyes, the individual layers of the retinal tissue can be seen as well-defined bands. Conversely, the characteristic defects caused by AMD or GA, for example, can be seen as a sharply demarcated region of degradation in the RPE and photoreceptor layers. In many eyes with GA, OCT images can show the wedge-shaped hyporeflective structures that can develop between the Brunch membrane and outer plexiform layer. Identification and monitoring of such structures can be useful in defining OCT boundaries of photoreceptor layers, which are important in clinical trials of therapies that aim to preserve the viability of the retinal layer in patients with AMD and GA.

By combining the segmentation of retinal layers in OCT with the metabolic mapping of fundus autofluorescence, morphologic alterations associated with functional change can be seen more clearly. Using specialized software, lesion areas seen in FAF images can be quantified and followed over time. Treatment effects, including areas of RPE regeneration that cover a lesion, can also be identified and recovery of RPE can be quantified by measuring the thickness of the retina.

In some embodiments, treatment leads to restoration of photoreceptors. RPE cells are involved in many processes critical for photoreceptor survival, including nutrient, water, and ion transport, light absorption, phagocytosis of shed photoreceptor outer segments (POS), re-isomerization of all-trans-retinal into 11-cis-retinal, which is crucial for the visual cycle, immune regulation, secretion of essential factors, and formation of the blood-retinal barrier. The RPE monolayer acts as a polarized metabolic gatekeeper between the PRs and the choroicapillaries (CC). The RPE has an apical to basolateral structural and functional polarity. On the apical side, RPE cells form multiple villi enabling direct contact with the POS and transport molecules such as glucose and vitamin A from the choroicapillaries to PRs. On the basal side, RPE cells transport metabolites such as CO2, lactate and water to the choroicapillaries and generate the underlying basal Bruch's membrane (BM) that separates the RPE from the choroid generating the blood-retinal barrier. On the lateral walls, adjoining RPE cells form tight junctions. Barrier function can be used to determine the potency of RPE cell cultures by measuring the tight junctions formed between the cells. RPE tight junctions limit paracellular movement of ions and water across the RPE monolayer and maintain the correct apico-basal distribution of RPE transporters. The RPE cell compositions disclosed herein display barrier function determined by the ability to generate Trans Epithelial Electrical Resistance (TEER) above 100Ω.

In addition, RPE cells secrete a variety of neurotrophic factors, such as fibroblast growth factors (bFGF and aFGF), ciliary neurotrophic factor (CNTF), pigment epithelium-derived factor (PEDF), brain-derived neurotrophic factor (BDNF), vascular endothelial growth factor (VEGF) and others, that help to maintain the structural integrity of choriocapillaris endothelium and photoreceptors. RPE cells also secrete anti-inflammatory cytokines such as transforming growth factor (TGF)-β, important in establishing the immune privileged properties of the eye. The RPE cells used in the RTA therapeutic cell compositions described herein are capable of secreting neurotrophic factors. The RPE cell compositions disclosed herein also demonstrate polarized PEDF and VEGF secretion which enhances RPE growth and blood vessel formation, respectively.

In certain embodiments, RPE cell implants provide long-lasting trophic support to degenerating retinal tissue by secreting these factors once implanted. This tropic support may act to attenuate retinal degradation and vision loss is some subjects. Trophic factors are known as cell survival and differentiation-promoting agents. Examples of trophic factors and tropic factor families include but are limited to, neurotrophins, the ciliary neurotrophic factor/leukemia inhibitory factor (CNTF/LIF) family, hepatocyte growth factor/scatter factor family, insulin-like growth factor (IGF) family, and the glial cell line-derived neurotrophic factor (GDNF) family. The RPE cells described herein may start secreting trophic factors immediately after administration or retinal grafting. In addition, a steady stream of neuroprotective support may start when the cells integrate in between the recipient cells and establish synaptic contacts with the subject's cells.

In some embodiments, the treatment/administration of RPE cells leads to pluripotent secretory effects of the RPE cells as described by J. Cell. Mol. Med. Vol 17, No 7, 2013 pp. 833-843, incorporated by reference in its entirety herein.

In some embodiments, the treatment may lead to restoration of the outer nuclear layer (ONL). The ONL (or layer of outer granules or external nuclear layer), is one of the layers of the vertebrate retina, the light-detecting portion of the eye. Like the inner nuclear layer, the outer nuclear layer contains several strata of oval nuclear bodies; they are of two kinds: rod and cone granules, so named on account of their being respectively connected with the rods and cones of the next layer.

The spherical rod granules are much more numerous, and are placed at different levels throughout the layer. Their nuclei present a peculiar cross-striped appearance, and prolonged from either extremity of each cell is a fine process; the outer process is continuous with a single rod of the layer of rods and cones; the inner ends in the outer plexiform layer in an enlarged extremity, and is imbedded in the tuft into which the outer processes of the rod bipolar cells break up. In its course it presents numerous varicosities.

The stem-like cone granules, fewer in number than the rod granules, are placed close to the membrana limitans externa, through which they are continuous with the cones of the layer of rods and cones. They do not present any cross-striation, but contain a pyriform nucleus, which almost completely fills the cell. From the inner extremity of the granule a thick process passes into the outer plexiform layer, and there expands into a pyramidal enlargement or foot plate, from which are given off numerous fine fibrils, that come in contact with the outer processes of the cone bipolars.

In some embodiments, the treatment may lead to restoration of the ellipsoid zone, as described elsewhere herein.

In some embodiments, the treatment may lead to restoration of the fovea of the retina.

In some embodiments, the treatment may lead to restoration or repair of the blood-retinal barrier (BRB), as described elsewhere herein.

In some embodiments, the restoring may lead to remodeling of the extracellular matrix (ECM). The ECM is a three-dimensional network consisting of extracellular macromolecules and minerals, such as collagen, enzymes, glycoproteins and hydroxyapatite that provide structural and biochemical support to surrounding cells. Because multicellularity evolved independently in different multicellular lineages, the composition of ECM varies between multicellular structures; however, cell adhesion, cell-to-cell communication and differentiation are common functions of the ECM.

The animal extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the ECM. Basement membranes are sheet-like depositions of ECM on which various epithelial cells rest. Each type of connective tissue in animals has a type of ECM: collagen fibers and bone mineral comprise the ECM of bone tissue; reticular fibers and ground substance comprise the ECM of loose connective tissue; and blood plasma is the ECM of blood.

In some embodiments, the restoring comprises one or more of reduced growth of geographic atrophy, improvement of visual acuity, improvement of reading speed, improvement of retinal structure, reductions in drusen (waste material removed by RPE cells), or stable engraftment of cells.

In embodiments, restoring includes reducing growth of geographical atrophy. In embodiments, reducing growth of geographical atrophy includes reducing the size of a geographical atrophy, such as reducing the total area of the atrophy. In embodiments, reducing growth of geographical atrophy includes reducing growth of an atrophic lesion. In embodiments, the atrophic lesion is isolated (independent of a primary GA). In embodiments, reducing growth of geographical atrophy includes reducing the growth rate of the geographical atrophy. In embodiments, reduction is compared to a control, such as an expected growth or growth rate, historical growth or growth rate, growth or growth rate in an untreated eye, an average growth or growth rate for subjects with a similar disease or disorder, or a growth or growth rate in a comparable subject.

In embodiments, growth of the geographical atrophy is less than about 98% of a control. In embodiments, growth of the geographical atrophy is less than about 95% of a control. In embodiments, growth of the geographical atrophy is less than about 90% of a control. In embodiments, growth of the geographical atrophy is less than about 85% of a control. In embodiments, growth of the geographical atrophy is less than about 80% of a control. In embodiments, growth of the geographical atrophy is less than about 75% of a control. In embodiments, growth of the geographical atrophy is less than about 70% of a control. In embodiments, growth of the geographical atrophy is less than about 65% of a control. In embodiments, growth of the geographical atrophy is less than about 60% of a control. In embodiments, growth of the geographical atrophy is less than about 50% of a control. In embodiments, growth of the geographical atrophy is less than about 40% of a control. In embodiments, growth of the geographical atrophy is less than about 30% of a control. In embodiments, growth of the geographical atrophy is less than about 25% of a control. In embodiments, growth of the geographical atrophy is less than about 20% of a control. In embodiments, growth of the geographical atrophy is less than about 10% of a control. In embodiments, growth of the geographical atrophy is between about 1% and about 99% of a control. In embodiments, growth of the geographical atrophy is between about 10% and about 90% of a control. Values can be any value or subrange within the recited ranges, including endpoints.

In embodiments, restoring includes improvement of visual acuity. In embodiments, improvement of visual acuity includes improvement over a control, such as pre-treatment (baseline) visual acuity. In embodiments, “improvement” includes a loss in visual acuity less than expected, such as less than a control, less than an untreated eye, less than historical rate of loss, less than an average rate of loss for a subject with a similar disease or disorder, and the like. In embodiments, improvement of visual acuity includes improved general vision. In embodiments, improvement of visual acuity includes improved color vision. In embodiments, improvement of visual acuity includes improvement in peripheral vision. In embodiments, improvement of visual acuity includes improvement in distance vision. In embodiments, improvement of visual acuity includes improvement in vision specific social functioning. In embodiments, improvement of visual acuity includes improvement in vision specific mental health. In embodiments, improvement of visual acuity includes improvement in vision specific dependency.

In embodiments, improvement in visual acuity is at least 5% improved compared to a control. In embodiments, improvement in visual acuity is at least 10% improved compared to a control. In embodiments, improvement in visual acuity is at least 20% improved compared to a control. In embodiments, improvement in visual acuity is at least 25% improved compared to a control. In embodiments, improvement in visual acuity is at least 30% improved compared to a control. In embodiments, improvement in visual acuity is at least 40% improved compared to a control. In embodiments, improvement in visual acuity is at least 50% improved compared to a control. In embodiments, improvement in visual acuity is at least 60% improved compared to a control. In embodiments, improvement in visual acuity is at least 70% improved compared to a control. In embodiments, improvement in visual acuity is at least 80%, 90%, 100% or more improved compared to a control. In embodiments, visual acuity is between about 5% and about 500% improved compared to a control. In embodiments, visual acuity is between about 5% and about 250% improved compared to a control. In embodiments, visual acuity is between about 5% and about 100% improved compared to a control. Improvement can be any value or subrange within the recited ranges, including endpoints.

In embodiments, restoring includes improvement of reading speed. In embodiments, improvement of reading speed includes improvement over a control, such as pre-treatment (baseline) reading speed. In embodiments, “improvement” includes a loss in reading speed less than expected, such as less than a control, e.g., less than an untreated eye, less than historical rate of loss, less than an average rate of loss for subjects with a similar disease or disorder, less than a rate of loss for a comparable subject, and the like.

In embodiments, improvement in reading speed is at least 5% improved compared to a control. In embodiments, improvement in reading speed is at least 10% improved compared to a control. In embodiments, improvement in reading speed is at least 20% improved compared to a control. In embodiments, improvement in reading speed is at least 25% improved compared to a control. In embodiments, improvement in reading speed is at least 30% improved compared to a control. In embodiments, improvement in reading speed is at least 40% improved compared to a control. In embodiments, improvement in reading speed is at least 50% improved compared to a control. In embodiments, improvement in reading speed is at least 60% improved compared to a control. In embodiments, improvement in reading speed is at least 70% improved compared to a control. In embodiments, improvement in reading speed is at least 80%, 90%, 100% or more improved compared to a control. In embodiments, reading speed is between about 5% and about 500% improved compared to a control. In embodiments, reading speed is between about 5% and about 250% improved compared to a control. In embodiments, reading speed is between about 5% and about 100% improved compared to a control. Improvement can be any value or subrange within the recited ranges, including endpoints.

In embodiments, restoring includes increasing thickness, preventing loss of thickness, or reduction in rate of loss of thickness of one or more regions of the retina. In embodiments, restoring includes increasing area, preventing loss of area, or reduction in rate of loss of area of one or more regions of the retina. In embodiments, restoring includes increasing volume, preventing loss of volume, or reduction in rate of loss of volume of one or more regions of the retina. In embodiments, the region of the retina includes in the vicinity of an atrophic region. In embodiments, the region of the retina may be one or more of the total retina, foveal center, subfoveal, central atrophy or lesion, peripheral atrophy or lesion, multiple lesion, RPE, External Limiting Membrane (ELM), Outer Nuclear Layer (ONL), Outer Plexiform Layer (OPL), Inner Nuclear Layer (INL), Inner Plexiform Layer (IPL), Ganglion Cell Layer (GCL), Retinal Nerve Fiber Layer (RNFL), Internal Limiting Membrane (ILM), Ellipsoid Zone (EZ), Inner/Outer segment of PR (IS/OS).

In embodiments, the thickness, area, or volume of the region of the retina is at least 5% improved compared to a control. In embodiments, the thickness, area, or volume of the region of the retina is at least 10% improved compared to a control. In embodiments, the thickness, area, or volume of the region of the retina is at least 20% improved compared to a control. In embodiments, the thickness, area, or volume of the region of the retina is at least 25% improved compared to a control. In embodiments, the thickness, area, or volume of the region of the retina is at least 30% improved compared to a control. In embodiments, the thickness, area, or volume of the region of the retina is at least 40% improved compared to a control. In embodiments, the thickness, area, or volume of the region of the retina is at least 50% improved compared to a control. In embodiments, the thickness, area, or volume of the region of the retina is at least 60% improved compared to a control. In embodiments, the thickness, area, or volume of the region of the retina is at least 70% improved compared to a control. In embodiments, the thickness, area, or volume of the region of the retina is at least 80%, 90%, 100% or more improved compared to a control. In embodiments, the thickness, area, or volume of the region of the retina is between about 5% and about 500% improved compared to a control. In embodiments, the thickness, area, or volume of the region of the retina is between about 5% and about 250% improved compared to a control. In embodiments, the thickness, area, or volume of the region of the retina is between about 5% and about 100% improved compared to a control. Improvement can be any value or subrange within the recited ranges, including endpoints.

In certain embodiments, treating or slowing the progression, maintain stasis of or reversing retinal disease is demonstrated by microperimetry assessed recovery of vision. Microperimetry, sometimes called Fundus related perimetry, is a type of visual field test which uses one of several technologies to create a “retinal sensitivity map” of the quantity of light perceived in specific parts of the retina in people who have lost the ability to fixate on an object or light source. Microperimetry-assessed recovery of vision comprises a correlation between retinal sensitivity on microperimetry and retinal anatomical changes/defect as compared to a baseline, an age-matched, sex-matched control, or a fellow eye of the subject. In certain embodiments, treating or slowing the progression, maintain stasis of or reversing retinal disease is demonstrated by microperimetry assessed recovery of vision, wherein there is a correlation of anatomical retinal changes or atrophic area found on spectral-domain optical coherence tomography (SD-OCT) with retinal sensitivity loss on macular integrity assessment (MAIA) microperimetry. See Invest Ophthalmol Vis Sci. 2017 May 1; 58(6):BIO291-BIO299. doi: 10.1167/iovs.17-21834, “Correlation Between Macular Integrity Assessment and Optical Coherence Tomography Imaging of Ellipsoid Zone in Macular Telangiectasia Type 2”; Mukherjee D. et al., which is herein incorporated by reference in its entirety.

In other embodiments, topographic maps, for example, orthogonal topographic (en face) maps, of the ellipsoid zone were generated from OCT volume scans, for example, Heidelberg Spectralis OCT volume scans (15×10° area, 30-μm B-scan intervals) or Zeiss Cirrus HD-OCT 4000 512×128 cube scans, to demonstrate treating or slowing the progression, maintain stasis of or reversing retinal disease, by comparing the maps to age-matched, sex-matched control, a baseline of the subject or a fellow eye of the subject. There is a correlation between organization of the EZ and retinal sensitivity. After administration of the RPE cells, the EZ zone organizes and retinal sensitivity improves. See for example, Retina, 2018 January; 38 Suppl 1:S27-S32. “Correlation Of Structural And Functional Outcome Measures In A Phase One Trial Of Ciliary Neurotrophic Factor In Type 2 Idiopathic Macular Telangiectasia,” Sallo F B, et al., which is incorporated by reference in its entirety.

In certain embodiments, treating or slowing the progression, maintain stasis of or reversing retinal disease is demonstrated by OCT-A, as compared to compared to age-matched, sex-matched controls, a baseline of the subject or a fellow eye before and after administration.

For example, using spectral-domain (SD)-OCT and OCT-A imaging and analyzing SD-OCT data using, for example, OCT EZ-mapping to obtain linear, area, and volumetric measurements of the EZ-retinal pigment epithelium (RPE) complex across the macular cube. OCT-A retinal capillary density can be measured using, for example, the Optovue Avanti split-spectrum amplitude-decorrelation angiography algorithm. EZ-RPE parameters are compared to age-matched, sex-matched controls, a baseline of the subject or a fellow eye.

In one embodiment, after administration, the EZ-RPE central foveal mean thickness improves, the EZ-RPE central foveal thickness improves, and EZ-RPE central subfield volume improves. EZ-RPE thickness, area, and volume are correlated with improved visual acuity to measure treatment response. Each of these measurements is inversely correlated with visual acuity. See, for example, methods outlined in, Invest Ophthalmol Vis Sci. 2017 Jul. 1; 58(9):3683-3689, “OCT Angiography and Ellipsoid Zone Mapping of Macular Telangiectasia Type 2 From the AVATAR Study,” Runkle A P., et al, which is incorporated by reference in its entirety.

In one embodiment, recovery, for example, is the subjective assessment that one or more of the following are becoming more organized, including the, external limiting membrane, myoid zone (inner segments of photoreceptors), ellipsoid zone (IS/OS Junction), outer segments of the photoreceptors, loss of drusen, and disappearance of reticular pseudo-drusen. Recovery may also comprise the subjective assessment that one or more of the basic foundational layers of the retina are becoming more organized. As used herein, the basic foundational layers of the retina becoming more organized comprise one or more of the external limiting membrane, myoid zone (inner segments of photoreceptors), ellipsoid zone (IS/OS Junction), and outer segments of the photoreceptors.

In one embodiment, the ellipsoid zone analysis demonstrates organization of the EZ by a decrease in the EZ volume as compared to an age-matched, sex-matched control, a baseline or a fellow eye. In another embodiment, the decrease in the EZ volume comprises at least 2% or at least 5% or at least 7% or at least 10%, or between 1 and 5% or between 1 and 10% or between 1 and 50% or between 10 and 50%. In another embodiment, the organization of the EZ is demonstrated, for example, by the decrease in volume of the structures of the EZ, see for example the comparison of the baseline and months 2 and 3. For example, the volume of the EZ is decreased by at least 2%, by at least 5%, by at least 10%. Each of the values or ranges recited herein may include any value or subrange therebetween, including endpoints.

In one embodiment, recovery comprises one or more of EZ-RPE central foveal mean thickness improvement, the EZ-RPE central foveal thickness improvement, and EZ-RPE central subfield volume improvement. EZ-RPE thickness, area, and volume are correlated with improved visual acuity to measure treatment response. Each of these measurements is inversely correlated with visual acuity.

In some embodiments, the improvement or restoration is measured by microperimetry.

In microperimetry, specific areas of the retina are stimulated with points of light, and the subject presses a button to acknowledge perception of the stimulus. In addition to identifying functional and nonfunctional areas, stimulus intensity can be varied to also identify the relative sensitivity of specific areas of the retina. The fundus can be monitored through an infrared camera and the sensitivity of the visual field can be mapped to the fundus photo and compared with images obtained with other techniques.

In certain embodiments, treating or slowing the progression, maintain stasis of or reversing retinal disease is demonstrated by microperimetry assessed recovery of vision, wherein microperimetry-assessed recovery of vision comprises a correlation between retinal sensitivity on microperimetry and retina anatomical changes/defect as compared to a baseline, an age-matched, sex-matched control, or a fellow eye of the subject. In certain embodiments, treating or slowing the progression, maintaining stasis of or reversing retinal disease is demonstrated by microperimetry-assessed recovery of vision, wherein there is a correlation of anatomical retinal changes or atrophic area found on spectral-domain optical coherence tomography (SD-OCT) with retinal sensitivity loss on macular integrity assessment (MAIA) microperimetry. See Invest Ophthalmol Vis Sci. 2017 May 1; 58(6):BIO291-BIO299. doi: 10.1167/iovs.17-21834, “Correlation Between Macular Integrity Assessment and Optical Coherence Tomography Imaging of Ellipsoid Zone in Macular Telangiectasia Type 2”; Mukherjee D. et al., which is herein incorporated by reference in its entirety.

The RPE cells may be transplanted in various forms. For example, the RPE cells may be introduced into the target site in the form of single cell suspension, with matrix or adhered onto a matrix or a membrane, extracellular matrix or substrate such as a biodegradable polymer or a combination. The RPE cells may also be printed onto a matrix or scaffold. The RPE cells may also be transplanted together (co-transplantation) with other retinal cells, such as with photoreceptors. The effectiveness of treatment may be assessed by different measures of visual and ocular function and structure, including, among others, best corrected visual acuity (BCVA), retinal sensitivity to light as measured by perimetry or microperimetry in the dark and light-adapted states, full-field, multi-focal, focal or pattern electroretinography 5 ERG), contrast sensitivity, reading speed, color vision, clinical biomicroscopic examination, fundus photography, optical coherence tomography (OCT), fundus auto-fluorescence (FAF), infrared and multicolor imaging, fluorescein or ICG angiography, adoptive optics and additional means used to evaluate visual function and ocular structure.

In some embodiments, the cell therapeutic agent is implanted into the subretinal space using a delivery device. In some embodiments, the delivery device comprises a needle, a capillary and a tip. In embodiments, the delivery device comprises a needle with an outer diameter of about 0.63 mm and an inner diameter of about 0.53 mm, a capillary with an outer diameter of about 0.5 mm and an inner diameter of about 0.25 mm, and a tip with an outer diameter of about 0.12 mm and an inner diameter of about 0.07 mm.

In another aspect is provided a method of assessing the progression of retinal disease or disorder as set forth, described or illustrated herein.

In an aspect a method of producing a cell therapeutic as set forth, described or illustrated herein is provided.

In an aspect is provided a method of assessing and improving the vision according to an assessment measure set forth, described or illustrated herein. In embodiments, the assessment is one or more of: reduced growth of geographic atrophy, visual acuity, reading speed, retina structure, reductions in drusen, or stable engraftment of cells. In embodiments, the assessment is reduced growth of geographic atrophy. In embodiments, the assessment is visual acuity. In embodiments, the assessment is reading speed. In embodiments, the assessment is retina structure. In embodiments, the assessment is reductions in drusen. In embodiments, the assessment is stable engraftment of cells.

For the methods provided herein, in embodiments the method results in minimal or no delayed inflammation of rejection of implanted cells. In embodiments, the method results in minimal rejection of implanted cells. In embodiments, the method results in delayed inflammation of rejection of implanted cells.

For the methods provided herein, in embodiments, the method includes a patient population, patient characteristic or patient demographic as set forth, described or illustrated herein. In embodiments, the method includes a patient population as set forth, described or illustrated herein. In embodiments, the method includes a patient characteristic as set forth, described or illustrated herein. In embodiments, the method includes a patient demographic as set forth, described or illustrated herein.

In some embodiments, the method may further comprise selecting a patient (subject), patient population, patient characteristic, or patient demographic as set forth, described or illustrated herein. In some embodiments, the patient population suffers from a retinal disease origin or related to RPE damage, malfunction or loss from various pathologies. In some embodiments, the patient population suffers from a retinal disease condition selected from the group consisting of Dry AMD, retinitis pigmentosae, usher syndrome, vitelliform maculopathy, Stargardt disease, retinal detachment, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, pathologic myopia, retinitis pigmentosa, and macular degeneration. In embodiments, a patient is chosen who suffers from AMD. In embodiments, the patient suffers from dry AMD. In embodiments, the patient suffers from wet AMD.

In addition to the above-mentioned disease, a non-limiting list of diseases for which the effects of treatment may be measured in accordance with the methods described also comprises lebers congenital amaurosis, hereditary or acquired macular degeneration, age related macular degeneration (AMD), geographic atrophy (GA), Best disease, retinal detachment, gyrate atrophy, choroideremia, pattern dystrophy as well as other dystrophies of the RPE, RPE and retinal damage due to damage caused by any one of photic, laser, inflammatory, infectious, radiation, neo vascular or traumatic injury. According to a particular embodiment, the disease is dry AMD. According to another embodiment, the disease is GA.

In embodiments, the method includes selecting a patient with dry AMD. In embodiments, the method includes selecting a patient with advanced dry AMD. In embodiments, the method includes selecting a patient with dry AMD with GA. In embodiments, the method includes selecting a patient with advanced dry AMD with GA. In embodiments, the method includes selecting a patient with best corrected visual acuity (BCVA) of 20/200 or worse. In embodiments, the method includes selecting a patient with best corrected visual acuity (BCVA) of 20/63 to 20/250. In embodiments, the method includes selecting a patient with best corrected visual acuity (BCVA) of better than 20/250. In embodiments, the method includes selecting a patient with best corrected visual acuity (BCVA) of better than 20/100. In embodiments, the method includes selecting a patient with best corrected visual acuity (BCVA) of better than 20/63. In embodiments, the method includes selecting a patient with central GA include the macula area. In embodiments, the method includes selecting a patient with central GA without including the macula area. In embodiments, the method includes selecting a patient with peripheral GA. In embodiments, the method includes selecting a patient with central and peripheral GA. In embodiments, the method includes selecting a patient with GA size of about 0.2 mm² or more.

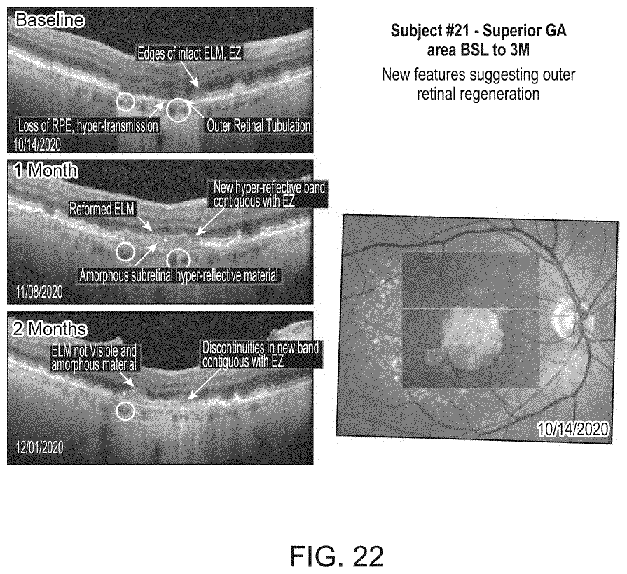

The findings described herein support a unique perspective by which an RPE cell transplant in accordance with the teachings of the present invention can replace or rescue retinal cells in patients who suffer from retinal lesions or degeneration. Importantly, in peripheral areas of incomplete RPE and outer retinal atrophy (iRORA), away from the primary atrophy lesion, examples of extensive resolution following OpRegen transplant are disclosed (see, for example, FIG. 21).

### Devices

For the methods provided herein, in embodiments, the method includes a device or apparatus as described, presented or set forth herein.

In an aspect, devices and/or compositions are provided for use in the methods, the devices and compositions as set forth, described or illustrated herein.

In some embodiments, the present disclosure provides a delivery device for use with any of the methods described herein.

In some embodiments, the device comprises a needle, a capillary and a tip. In some embodiments, the device comprises a needle with an outer diameter of about 0.63 mm and an inner diameter of about 0.53 mm, a capillary with an outer diameter of about 0.5 mm and an inner diameter of about 0.25 mm, and a tip with an outer diameter of about 0.12 mm and an inner diameter of about 0.07 mm.

In embodiments, the compositions of matter, methods and devices can utilize product candidates that are allogeneic (“off-the-shelf”). For example, that can mean that the material is derived from cell lines, not from individual patients, facilitating large-scale production and lower production costs than patient-specific treatments

The methods, device, compositions of matter, etc., can include those set forth in the accompanying Figures, which are incorporated herein by reference.

### EXAMPLES

### Example 1: Interim Results from 24-Patient Phase 1/2a Clinical Study of OpRegen

OpRegen was evaluated in a Phase 1/2a open-label, dose escalation safety and efficacy study of a single injection of human retinal pigment epithelium cells derived from an established pluripotent cell line and transplanted subretinally in patients with advanced dry AMD with GA. The study enrolled 24 patients into 4 cohorts. The first 3 cohorts enrolled subjects with advanced stages of the disease. All 12 subjects of the first 3 cohorts were legally blind with best corrected visual acuity (BCVA) of 20/200 or worse with advanced GA (size of about 17 mm²). The fourth cohort enrolled 12 subjects presented at earlier stages of the disease compared to cohorts 1-3, with better vision (vision from 20/63 to 20/250) and smaller areas of GA (maximum of 1 mm²). Cohort 4 also included subjects treated with a new “thaw-and-inject” (TAI) formulation of OpRegen, which can be shipped directly to sites and used immediately upon thawing, removing the complications and logistics of having to use a dose preparation facility. The first 3 subjects of cohort 4 were treated with the previous formulation and the last 9 subjects of cohort 4 were treated with “TAI” formulation. The primary objective of the study was to evaluate the safety and tolerability of OpRegen as assessed by the incidence and frequency of treatment emergent adverse events. Secondary objectives were to evaluate the preliminary efficacy of OpRegen treatment by assessing the changes in ophthalmological parameters measured by various methods of primary clinical relevance. Additional objectives include the evaluation of the safety of delivery of OpRegen using the Gyroscope SDS.

The 12 subjects treated in Cohort 4 had a better baseline vision and smaller areas of geographic atrophy (GA). In Cohort 1-3, subjects who were legally blind at baseline, visual acuity (VA) reductions occurred as expected due to progressive GA. In Cohort 4, subjects with smaller areas of GA and higher baseline best corrected visual acuity (BCVA), improved or sustained BCVA was observed in 11/12 (92%) subjects as of their last visit (range of −7 to +19 ETDRS letters). OpRegen was well-tolerated in all treated subjects (N=24), including 2 subjects with less immunosuppression (COVID or other health conditions). No acute or delayed inflammation and no sustained increased intraocular pressure (IOP) were observed. All subjects reported at least one adverse event (AE), however, the majority of AEs were mild (87%). AEs in eye-related disorders system (n=165 events) include: n=136 in Pars Plana Vitrectomy (PPV) treated subjects (n=17 subjects; 54.7 years F/U), and n=29 in Orbit SDS treated subjects (n=7 subjects; 6.9 years F/U). Sustained subretinal pigmentation suggested multi-year durability of OpRegen. Improved anatomy and function continue to be observed in some subjects, including: reduction in drusen, photoreceptor and RPE layer restoration, localized slowing of GA progression in treated areas, better visual acuity via ETDRS scores and reading speed, and improved NEI Visual Function Questionnaire (VFQ-25) scores (National Eye Institute Visual Functioning Questionnaire-25 (NEI VFQ-25) Version 2000—Interviewer Administered Format). Post-treatment surgical interventions occurred in four cases (5 events in 4 subjects) including; three epiretinal membranes (ERM) were surgically peeled (ERM were observed in 15 out of 17 subjects, most were clinically insignificant), retinal detachment (RD) was observed in 2 out of 17 subjects receiving cells via PPV retinotomy, and treatment-responsive choroidal neovascularization (CNV) was observed in three Orbit SDS treated subjects, all of whom received a single administration of an approved anti-VEGF. OpRegen TAI formulation was administered in 7 Orbit SDS and 2 PPV-treated subjects. Slow resorption of subretinal fluid, without sequelae, was observed in 4 Orbit SDS/TAI treated subjects. Assessments of clinical benefit are ongoing and are utilizing detailed OCT analyses in addition to standard FAF measurements. Long-term follow-up of subjects is ongoing.

As part of an ongoing effort to administer the minimally effective dose and duration of immunosuppressive therapy, immunosuppression was utilized only during the perioperative period of approximately 3 months in Cohort 4 subjects. Notably, one OpRegen patient who received a modified immunosuppressive regimen at baseline, which included no tacrolimus and only mycophenolate mofetil, did not show any signs of acute or delayed inflammation or rejection of OpRegen cells 4.5 months after transplant. One patient was diagnosed with COVID shortly after treatment and all immunosuppression was halted and reinstated once the patient was asymptomatic. This second patient similarly showed no signs of acute or delayed inflammation or rejection of OpRegen cells 4.5 months post-surgery. Other than the reduced regimens described above, immunosuppressants were discontinued as scheduled, typically within 90 days post-operatively, and no cases of acute or delayed rejection or inflammation due to OpRegen were reported.

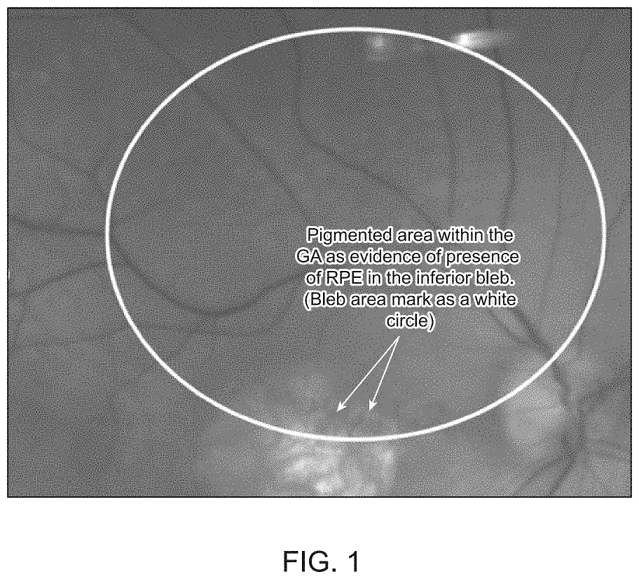

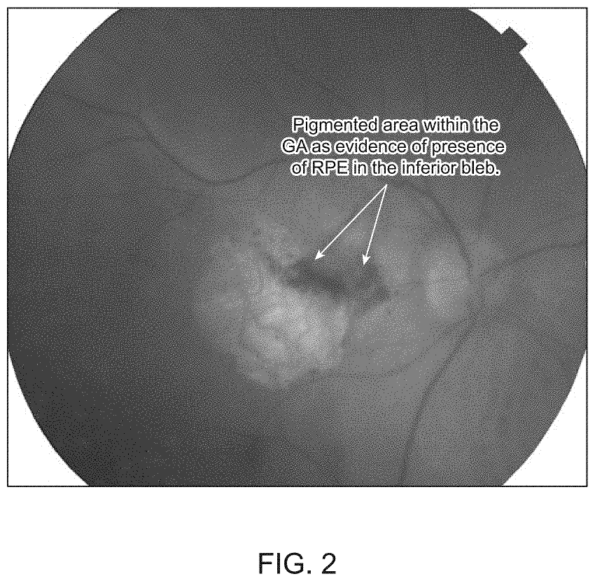

Nine subjects were treated with a new “thaw-and-inject” (TAI) formulation of OpRegen and 7 were treated using the Gyroscope Orbit™ Subretinal Delivery System (Orbit SDS). Representative FP images of pigmented areas within GA of treated eyes are shown at 3 months (FIG. 1) and 9 months (FIG. 2) after treatment. Pigmented area is evidence of the presence of RPE cells within the GA.

Overall, 11/12 (92%) of the Cohort 4 subjects' treated eyes were at or above baseline visual acuity at 4.5 months to >3 years post-transplant. Improvements in best corrected visual acuity (BCVA) reached up to +19 letters on the Early Treatment Diabetic Retinopathy Study (ETDRS) chart. In contrast, 11/12 (92%) of the subjects' untreated eyes were below baseline entry values at the same time points. Among the newly reported data, three (50%) of the more recently treated Cohort 4 subjects exhibited marked improvements in BCVA ranging from +7 to +16 letters at their last scheduled assessments of at least 4.5 months. Two additional Cohort 4 subjects experienced a gain of 2 letters from their baseline values. One patient measured 7 letters below baseline. Previously reported structural improvements in the retina and decreases in drusen density in some subjects have continued. Evidence of durable engraftment of OpRegen RPE cells extended to more than 5 years in earliest treated subjects. A trend towards slower GA progression in treated compared to fellow eyes continued. Overall, OpRegen was well tolerated with no unexpected adverse events or serious adverse events.

The data in Tables 1, 2, and 3 below summarize the changes to the recoded values for five subjects in cohort 4 (14, 15, 13, 16 & 17). For the vision categories, all five subjects saw improvement. The average change of recoded values for all five subjects combined for the vision categories was 18%.

A Cohort 4 subject with evidence of retinal restoration and confirmed history of GA growth, which was first reported at 9 months, continued at month 23 to have an area of GA smaller than at baseline. This subject also experienced additional improvement in BCVA from 9 to 23 months post-treatment, while the untreated eye has experienced further reduction in visual acuity.

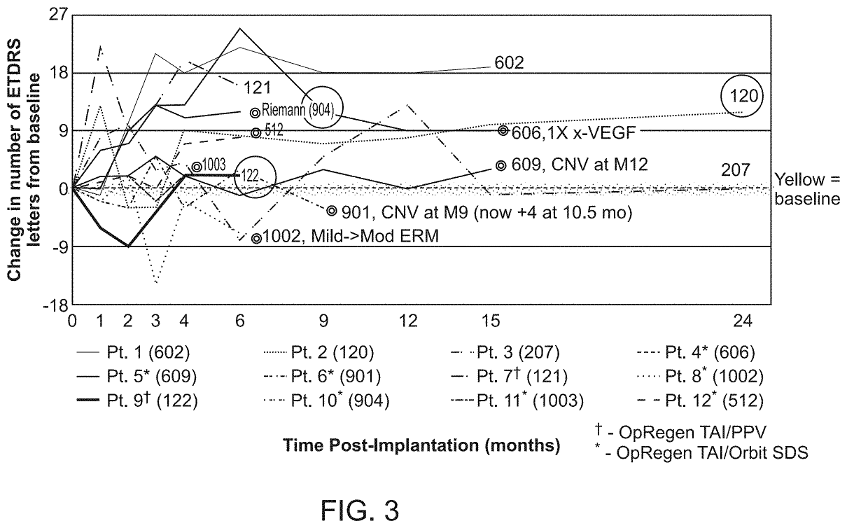

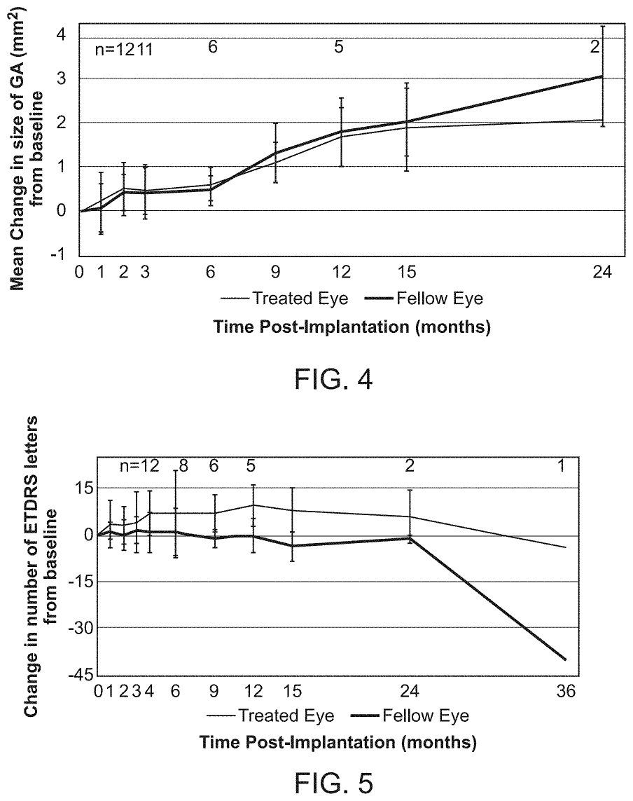

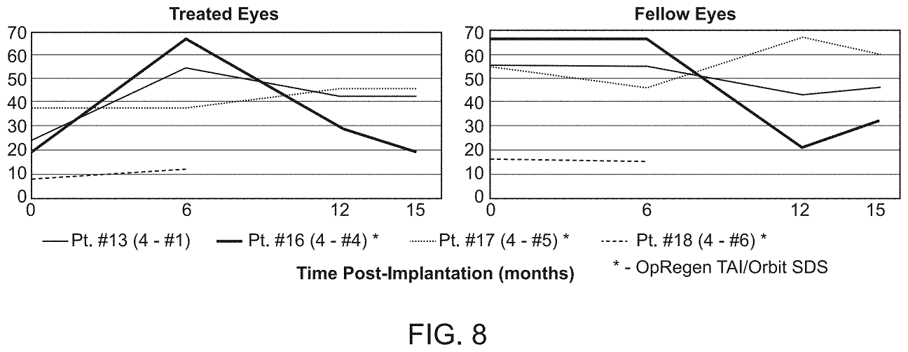

Individual changes in visual acuity over time (1 to 24 months) for Cohort 4 are shown in FIG. 3 (measured by change in number of ETDRS letters from baseline) and FIG. 8 (measured by reading speed). Mean change in visual acuity (measured by change in number of ETDRS letters from baseline) is shown in FIG. 5. Mean change in size of GA in treated eyes is shown in FIG. 4.

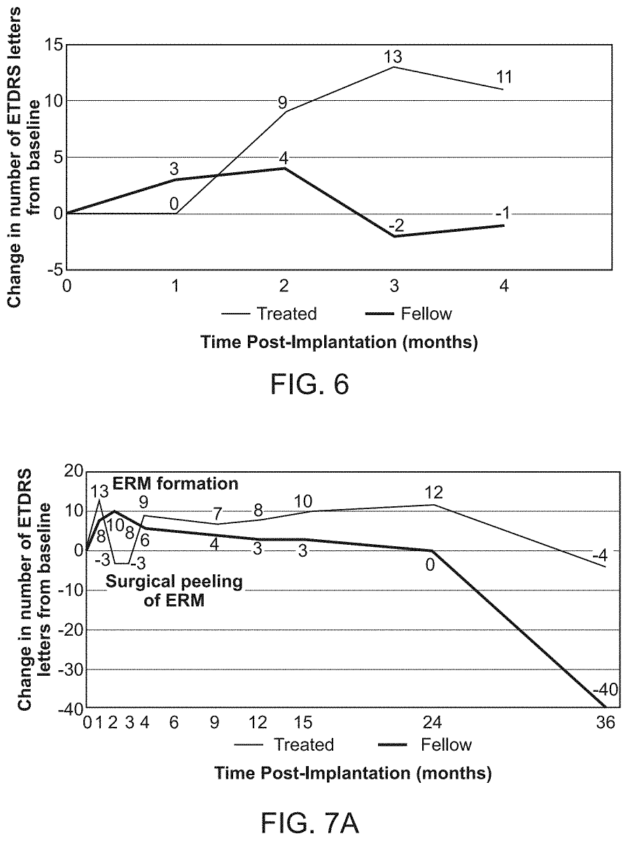

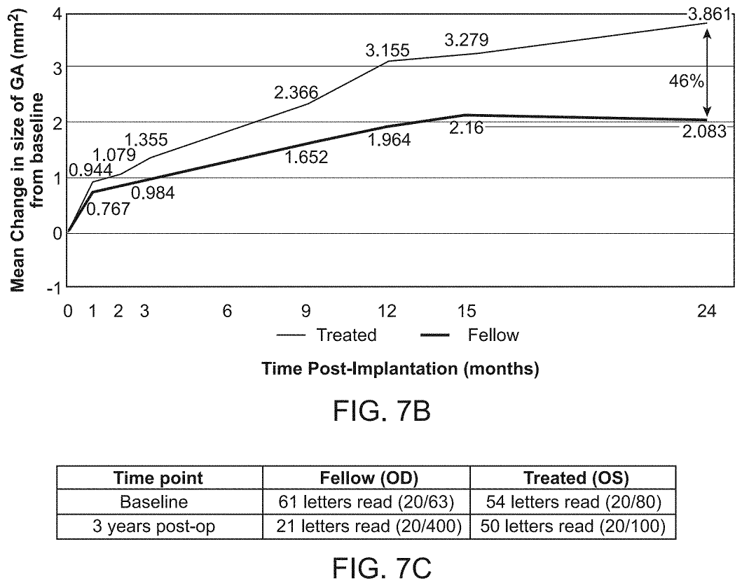

Data for individual subjects are shown in FIGS. 6 and 7A-7C.

TABLE 1
Total percentage change averages across all subjects and categories.
All Vision/Ocular

| | Category/Patient | | | | | | | | | | | | | | | | |
|---|---|---|---|---|---|---|---|---|---|---|---|---|---|---|---|---|---|
| | 14 | | | 15 | | | 13 | | | 16 | | | 17 | | | Aver- | |
| | VISIT/Total Change % -> | | | | | | | | | | | | | | | age | Im- |
| | | | % | | | % | | | % | | | % | | | % | Change | prove- |
| | V-1 | V-17 | Change | V-1 | V-17 | Change | V-1 | V-17 | Change | V-1 | V-17 | Change | V-1 | V-17 | Change | (All) | ment |
| General | 40.0 | 80.0 | 100.0 | 80.0 | 60.0 | −25% | 40.0 | 80.0 | 100% | 40.0 | 60.0 | 50% | 40.0 | 60.0 | 50% | 55% | 4 of 5 |
| Vision | | | | | | | | | | | | | | | | | |
| Ocular | 100.0 | 87.5 | −13% | 12.5 | 75.0 | 500% | 100.0 | 100.0 | 0% | 100.0 | 75.0 | −25% | 100.0 | 100.0 | 0% | 93% | 2 of 5 |
| Pain | | | | | | | | | | | | | | | | | |
| Near | 50.0 | 56.7 | 13% | 40.0 | 56.7 | 42% | 48.3 | 56.7 | 17% | 40.0 | 66.7 | 67% | 48.3 | 50.0 | 3% | 28% | 5 of 5 |
| Activities | | | | | | | | | | | | | | | | | |
| Distance | 33.3 | 62.5 | 88% | 41.7 | 41.7 | 0% | 83.3 | 75.0 | −10% | 41.7 | 83.3 | 100% | 66.7 | 66.7 | 0% | 36% | 3 of 5 |
| Activities | | | | | | | | | | | | | | | | | |
| Vision | 62.5 | 62.5 | 0% | 87.5 | 100.0 | 14% | 100.0 | 100.0 | 0% | 87.5 | 100.0 | 14% | 62.5 | 75.0 | 20% | 10% | 3 of 5 |
| Specific: | | | | | | | | | | | | | | | | | |
| Social | | | | | | | | | | | | | | | | | |
| Function- | | | | | | | | | | | | | | | | | |
| ing | | | | | | | | | | | | | | | | | |
| Vision | 62.5 | 87.5 | 40% | 12.5 | 37.5 | 200% | 62.5 | 68.8 | 10% | 18.8 | 37.5 | 100% | 50.0 | 81.3 | 63% | 83% | 5 of 5 |
| Specific: | | | | | | | | | | | | | | | | | |
| Mental | | | | | | | | | | | | | | | | | |
| Health | | | | | | | | | | | | | | | | | |
| Vision | 75.0 | 62.5 | −17% | 12.5 | 100.0 | 700% | 62.5 | 75.0 | 20% | 50.0 | 75.0 | 50% | 50.0 | 75.0 | 50% | 161% | 4 of 5 |
| Specific: | | | | | | | | | | | | | | | | | |
| Role | | | | | | | | | | | | | | | | | |
| Difficul- | | | | | | | | | | | | | | | | | |
| ties | | | | | | | | | | | | | | | | | |
| Vision | 83.3 | 100.0 | 20% | 91.7 | 66.7 | −27% | 100.0 | 100.0 | 0% | 41.7 | 58.3 | 40% | 83.3 | 91.7 | 10% | 9% | 3 of 5 |
| Specific: | | | | | | | | | | | | | | | | | |
| Depend- | | | | | | | | | | | | | | | | | |
| ency | | | | | | | | | | | | | | | | | |
| Driving | N/A | N/A | N/A | 0.0 | 2.5 | N/A | N/A | N/A | N/A | 58.3 | 41.7 | −29% | 83.3 | 75.0 | −10% | −19% | 0 |
| Color | 100.0 | 100.0 | 0% | 100.0 | 100.0 | 0% | 100.0 | 100.0 | 0% | 100.0 | 100.0 | 0% | 100.0 | 100.0 | 0% | 0% | No |
| Vision | | | | | | | | | | | | | | | | | |
| Peripheral | 100.0 | 50.0 | −50% | 50.0 | 100.0 | 100% | 100.0 | 100.0 | 0% | 50.0 | 75.0 | 50% | 100.0 | 100.0 | 0% | 20% | 2 of 5 |
| Vision | | | | | | | | | | | | | | | | | |
| Per | 70.7 | 74.9 | 6% | 52.8 | 73.8 | 40% | 79.7 | 85.5 | 7% | 57.08 | 70.2 | 23% | 71.3 | 79.5 | 12% | 18% | |
| Patient | | | | | | | | | | | | | | | | | |
| Averages | | | | | | | | | | | | | | | | | |

TABLE 2
Total percentage change averages across all subjects and categories.
General Health

| | Category/Patient | | | | | | | | | | | | | | | |
|---|---|---|---|---|---|---|---|---|---|---|---|---|---|---|---|---|
| | 14 | | | 15 | | | 13 | | | 16 | | | 17 | | | Average |
| | VISIT/Total Change % -> | | | | | | | | | | | | | | | Change |
| | V-1 | V-17 | CHG | V-1 | V-17 | CHG | V-1 | V-17 | CHG | V-1 | V-17 | CHG | V-1 | V-17 | CHG | (All) |
| General Health | 75 | 75 | 0 | 50 | 25 | −50% | 100 | 100 | 0% | 50 | 75 | 50% | 50 | 50 | 0% | 0 |

TABLE 3
This table shows how many of the five subjects
in cohort 4 showed improvement for each category.

| | # of | | |
|---|---|---|---|
| | Subject | | |
| Category | out of 5 | % | |
| General Vision | 4 | 80% | of subjects showed improvement |
| Ocular Pain | 2 | 40% | of subjects showed improvement |
| Near Activities | 5 | 100% | of subjects showed improvement |
| Distance Activities | 3 | 60% | of subjects showed improvement |
| Vision Specific: | 3 | 60% | of subjects showed improvement |
| Social | | | |
| Functioning | | | |
| Vision Specific: | 5 | 100% | of subjects showed improvement |
| Mental Health | | | |
| Vision Specific: | 4 | 80% | of subjects showed improvement |
| Role Difficulties | | | |
| Vision Specific: | 3 | 60% | of subjects showed improvement |
| Dependency | | | |
| Driving | 0 | 0% | of subjects showed improvement |
| | | | (only 2 subjects were driving at |
| | | | screening) |
| Color Vision | 0 | 0% | no change from baseline screening |
| Peripheral Vision | 2 | 40% | of subjects showed improvement |

A blank questionnaire (National Eye Institute Visual Functioning Questionnaire-25 (VFQ-25) Version 2000—Interviewer Administered Format) with all questions is hereby incorporated by reference. The questionnaire was administered at screening Visit 11, Visit 17, Visit 18, Visit 19, Visit 20, Visit 21 & Visit 22. Averaging the items as indicated in Table 4 generated VFQ-25 sub-scales.

TABLE 4

| | Number | Items to be averaged |
|---|---|---|
| Scale | of items | (after recoding per Table 2) |
| General Health | 1 | 1 |
| General Vision | 1 | 2 |
| Ocular Pain | 2 | 4, 19 |
| Near Activities | 3 | 5, 6, 7 |
| Distance Activities | 3 | 8, 9, 14 |
| Vision Specific: | | |
| Social Functioning | 2 | 11, 13 |
| Mental Health | 4 | 3, 21, 22, 25 |
| Role Difficulties | 2 | 17, 18 |
| Dependency | 3 | 20, 23, 24 |
| Driving | 3 | 15c, 16, 16a |
| Color Vision | 1 | 12 |
| Peripheral Vision | 1 | 10 |

Observations from the clinical trial data include quality of life improvements, improved reading speed, and improved microperimetry.

### Example 2: Retinal Restoration in Subjects with Dry AMD with GA

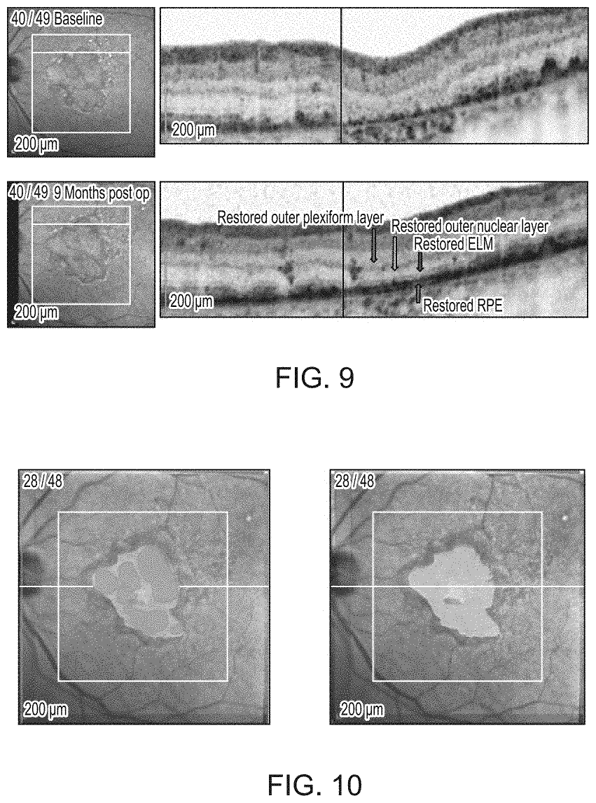

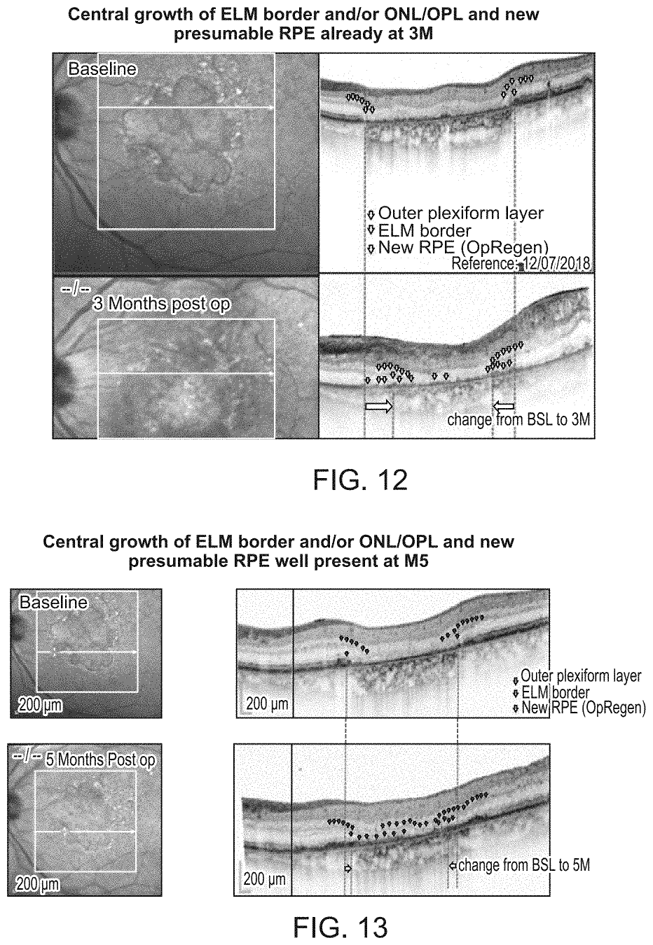

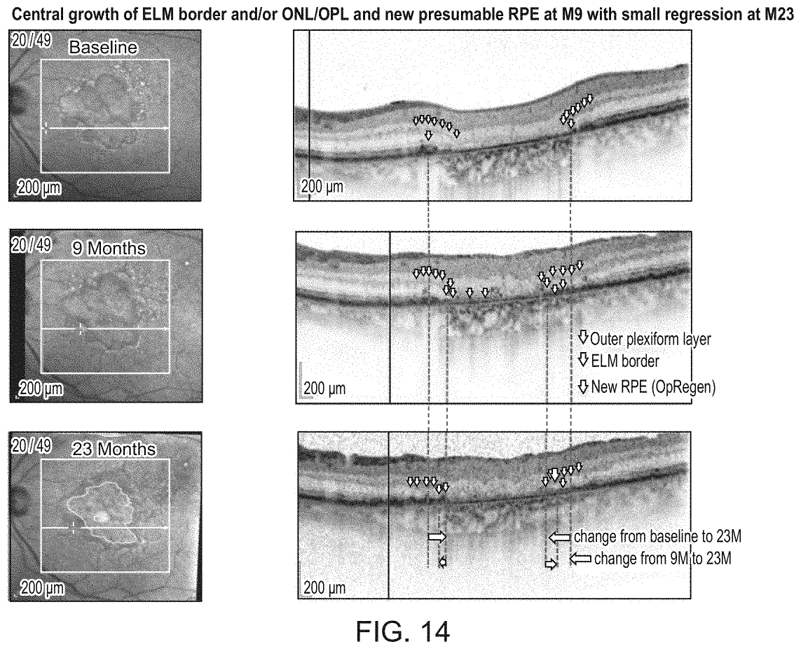

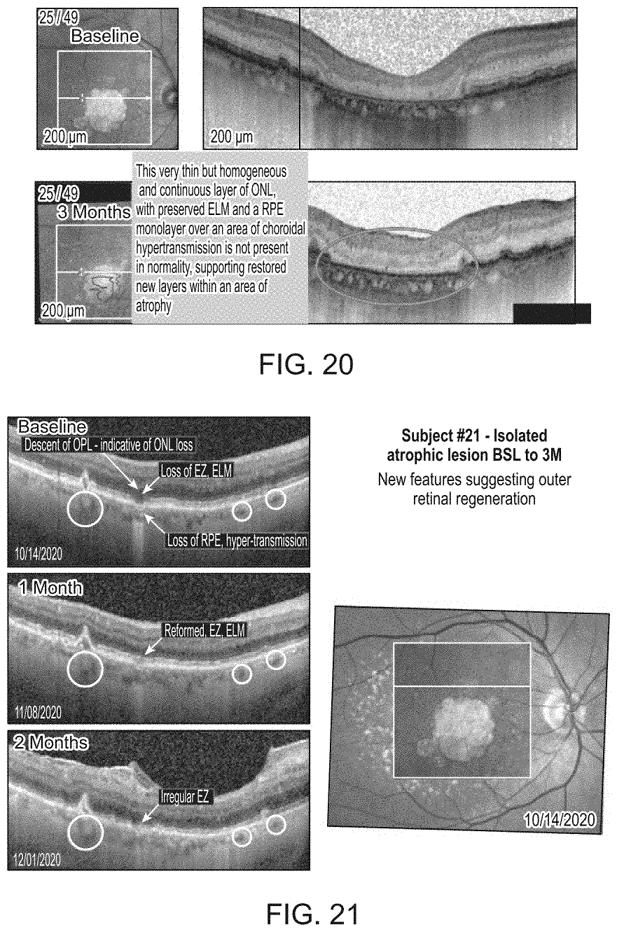

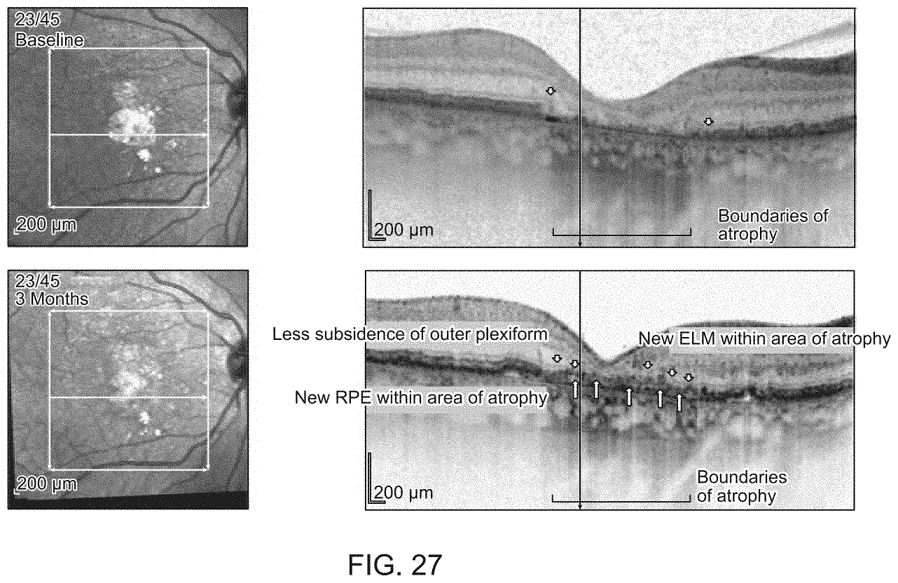

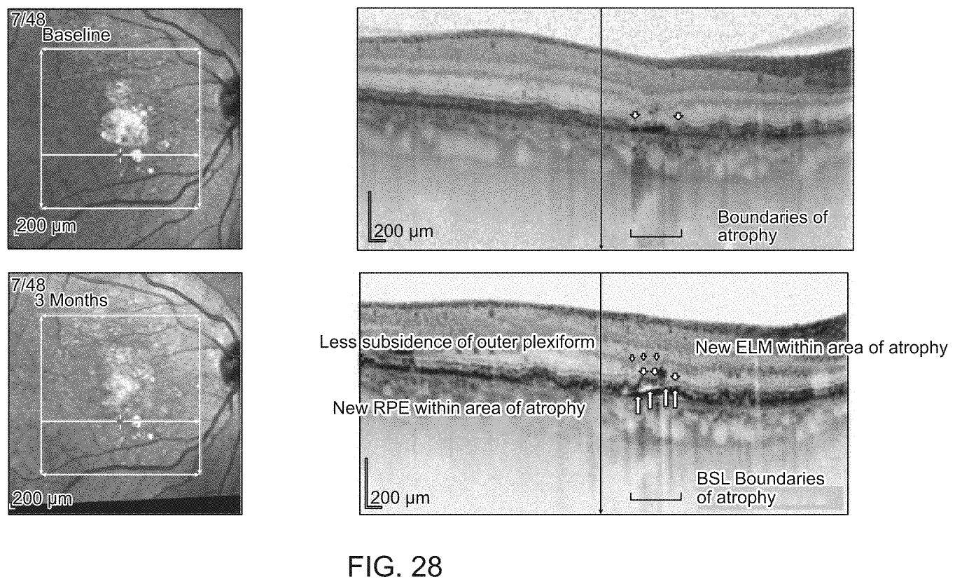

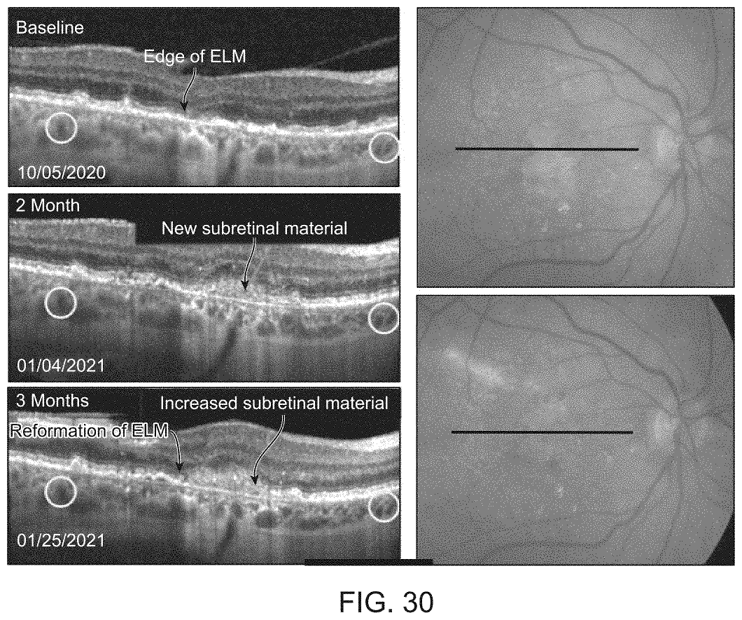

Retinal restoration is difficult to observe because the cells used herein do not autofluoresce under FAF, which is a common imaging technique used to measure GA boundaries. Measurement from IR has never been accepted as a method to assess atrophy boundaries. High-resolution OCT is an alternative to FAF for measuring GA lesion boundaries and the fine layers of the retina. Using OCT in this way is a slower, manual process with its own limitations, but it provides the ability to distinguish individual cell types within the retina, like the layers of a cake (ex: ONL, OPL, RPE). FIGS. 9, 12-14, 16, 18-22, 26-28, and 30 show a number of cross-sections and “aerial” perspectives of an area of atrophy at baseline and after treatment.

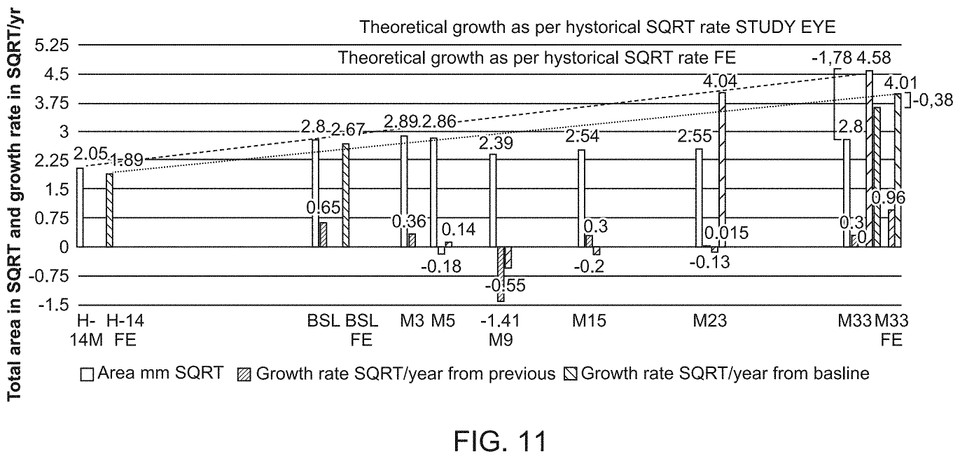

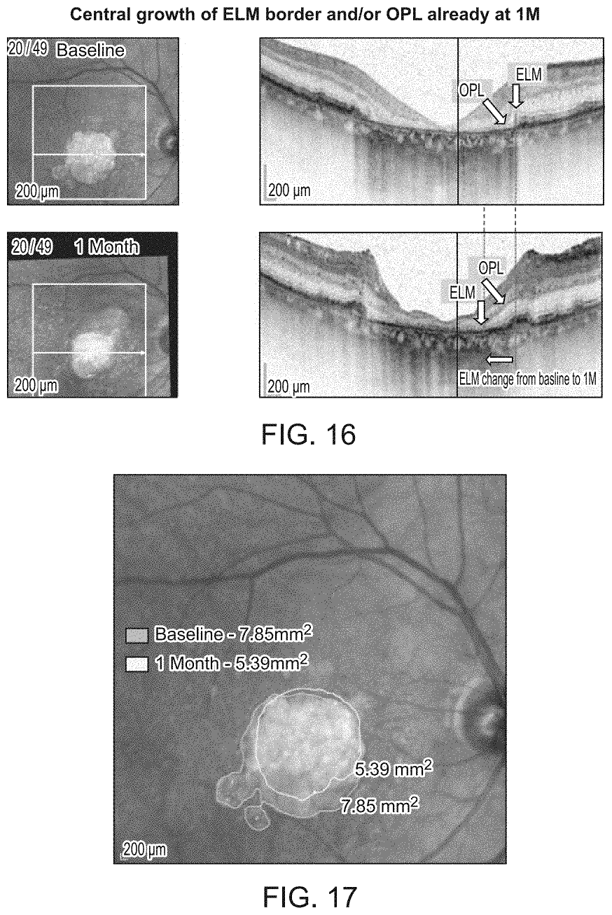

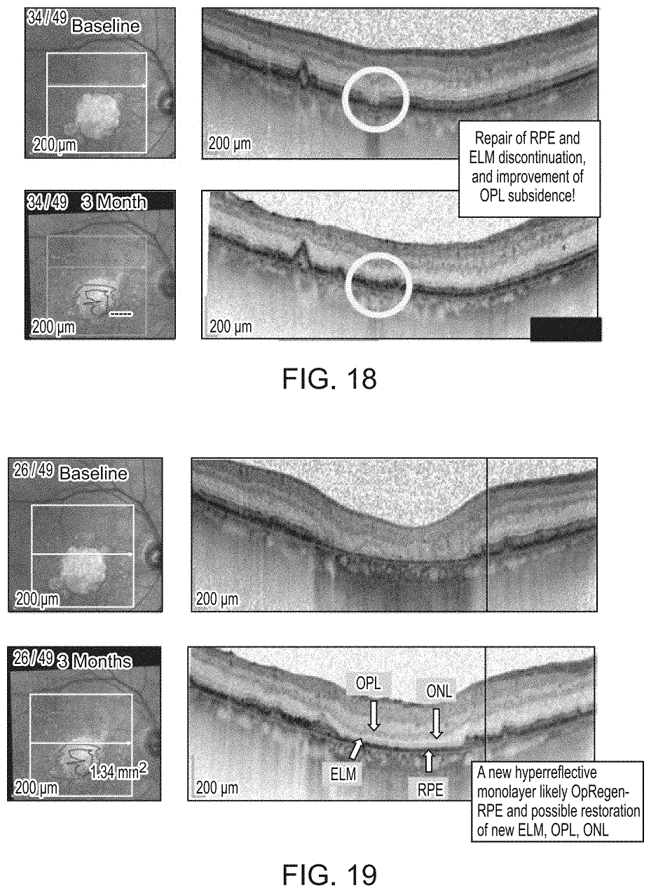

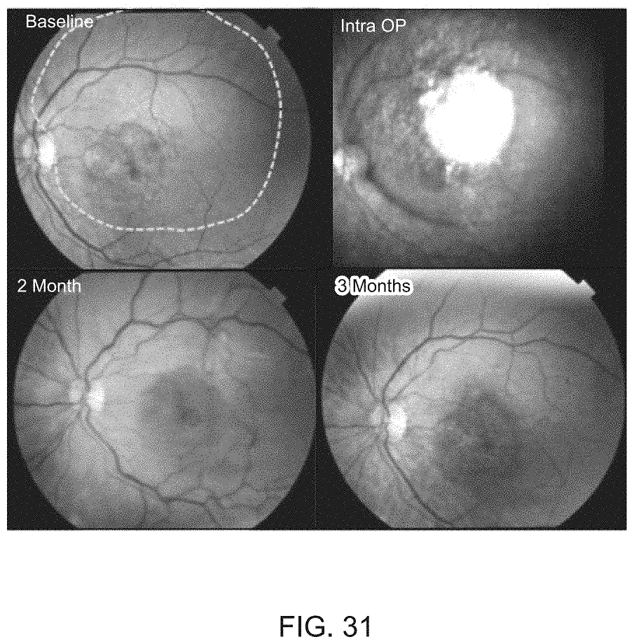

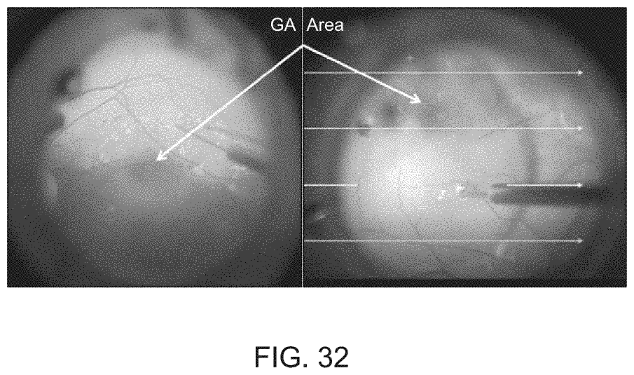

Subject 14 had anatomical improvement of OPL, ONL, ELM, RPE and outer retinal regeneration/restoration at 9 months and 23 months after treatment (FIGS. 9 to 15). Similarly, subject 21 had a reduction in GA boundaries and anatomical improvement and restoration of the ELM at 1 month (FIGS. 16 and 17), as well as almost complete restoration of a previously atrophic area (isolated from the primary GA), with regeneration of the missing layers and “disappearance” of the atrophic lesion (FIG. 18). Improvement was seen at 2 and 3 months post-treatment (FIG. 18-22). RPE delivery to the GA was observed in subject 14 during the treatment procedure, as well as 2 and 3 months post-treatment (FIG. 31).

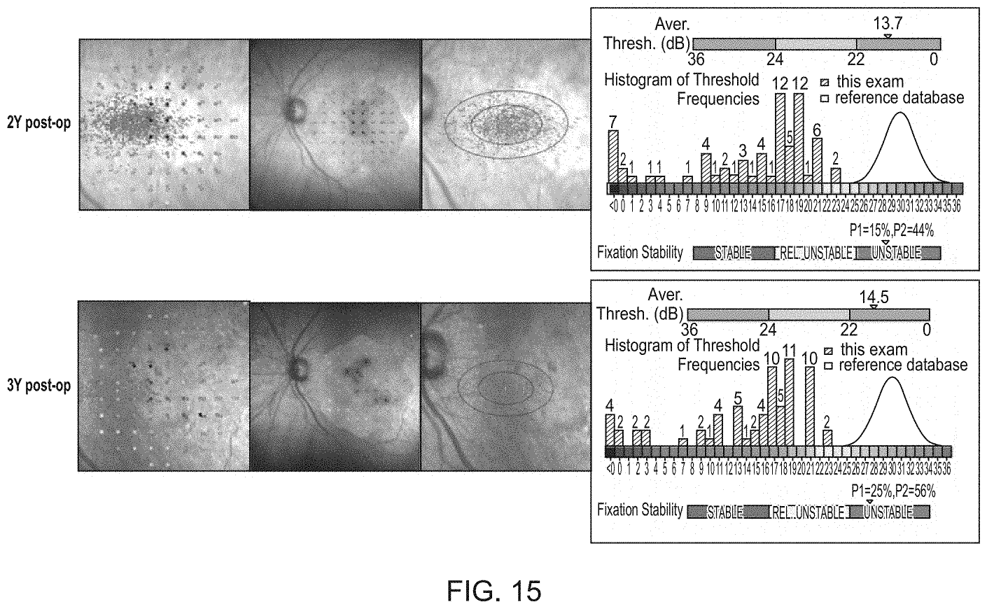

Microperimetry. FIG. 15 shows preliminary evidence that the area of restoration might also be functional (simply seeing tissue does not mean that tissue is active). Microperimetry involves flashing a pinpoint light onto the retina in order to “map” the area used for vision. Microperimetry data are difficult to collect, so they only exist for a few subjects at a small number of time points. However, they provide at least some evidence that Patient 14 has visual capability at the area of restoration.

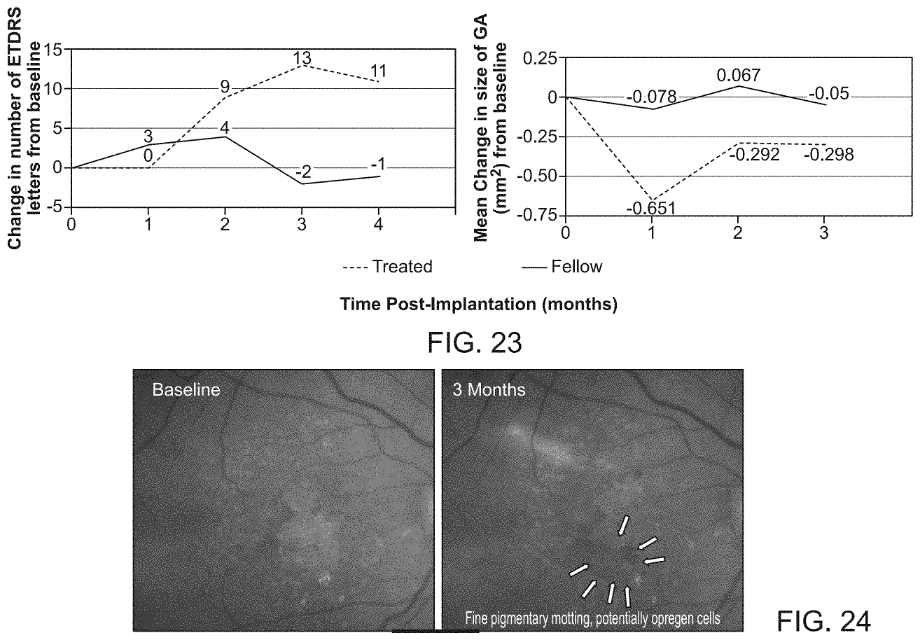

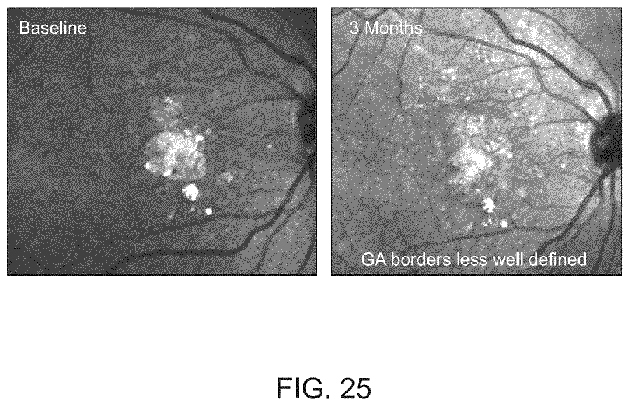

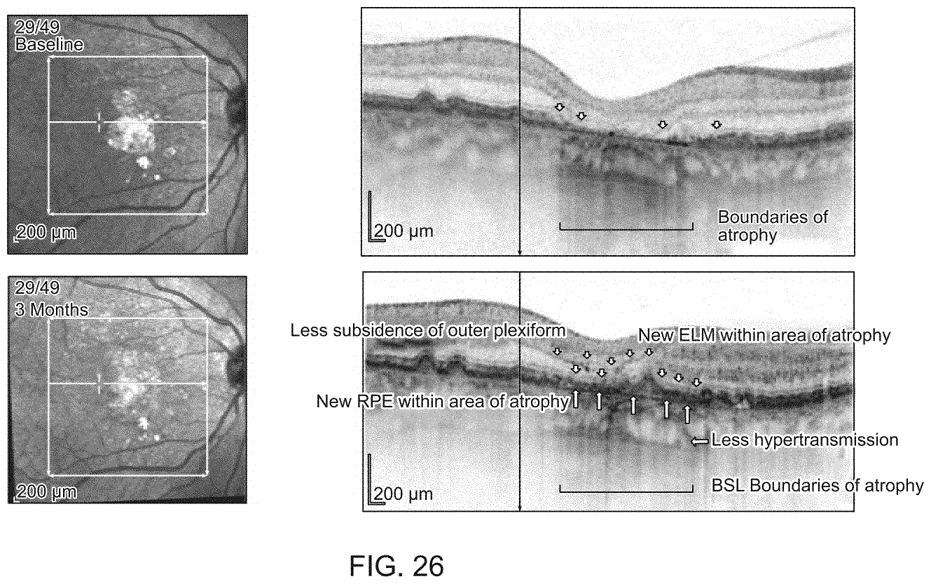

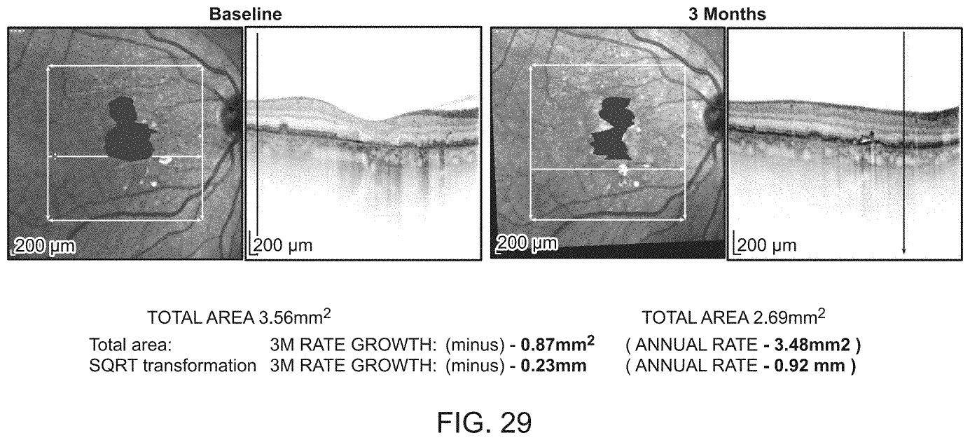

Subject 22 demonstrated improvement in visual acuity and GA size in the treated eye compared to the untreated eye (FIG. 23). Pigmentation at 3 months post-treatment in subject 22 indicated presence of RPE cells (FIG. 24). GA size as measured by IR imaging demonstrate reduction of boundaries of the GA at 3 months (FIG. 25), as do OCT measurements (FIGS. 26-30).

Subject 14 was followed out to 35 months. Discrete tissue layers were detectable at 23 months but were not present at 9 months. There were many examples of this phenomenon throughout the observation period and across the whole (peripheral) area of atrophy. Applicants measured the patient's GA growth rate for the year prior to treatment, allowing for extrapolation of the patient's GA size based on the untreated growth rate. The GA remained unchanged compared to baseline for 3 years, which was not expected to occur given the natural course of the disease (i.e. things get progressively worse). The patient's treated eye only recently fell below baseline, but remains far better than the contralateral eye which the patient no longer uses for vision. Subject 14 was the original case and shows durability of effect.

Subject 21 new finding. Similar observations were detected in a different patient as early as 2.5 months. Analysis was done on the outer retinal area only. Baseline showed expected GA/cRORA with loss of ELM, EZ at expected locations. Three weeks later, significant outer retinal changes were observed, including apparent partial reformation of ELM/EZ. Diffuse thickening of EZ and amorphous hyper-reflective sub-retinal material was present. At six weeks some EZ changes persisted, but EZ loss also occurred. A thickening of RPE/Bruch's was also observed.

Subject 22 new finding. Subject 22 is a woman who referred to her treatment experience as “life changing.” New material and extension of ELM in various locations were identified around the GA, as well as some small areas or “islands” of GA not connected to the main area. By 3 months, those islands had disappeared after treatment, supporting the claim that earlier intervention will lead to better clinical outcomes in dry AMD. Patient 22 was treated using the Orbit SDS.

Baseline showed central GA/cRORA with multifocal satellites. Expected loss of EZ/ELM/hyper-transmission was observed through RPE. At 4 weeks, there was macular hole formation with large sub-retinal fluid collection. Numerous deposits on RPE surface were identified on IR and OCT. At week 6, residual subretinal fluid, and new material was apparent on surface of RPE. PED was apparent with very hyper-reflective internal material, possible Type 1 CNV. By 3 months all subretinal fluid resolved, subretinal material persisted, and large central subretinal deposit appeared. There was new superior intra-retinal fluid. By 4 months, extension of ELM was noted at many locations. There was increased subretinal material. Retinal hemorrhage on fundus photo may correspond to areas of fluid and possible bud of Type 1 CNV through Bruch's. There was an overall expansion of RPE loss on FAF, but increased pigmentation and extension of ELM into boundaries of defined atrophy.

Summary

In subjects 14, 21, and 22, cases of restoration the transplanted cells covered the majority of the GA. Cell placement seems to be critical to achieving these outcomes, which had important implications for the Orbit evaluation. After seeing restoration in subject 14 (a patient who had complete coverage of the GA), surgeons made a greater effort to deliver the cells across the GA in the final 7 subjects. In the final 4 Orbit subjects, only one successfully deposited the cells across the GA, despite being in the hands of highly trained surgeons. In contrast, both of the PPV-accessed procedures were successfully able to accomplish this (PPV is more flexible in this regard). In the third case (Pt. #22), using Orbit, a partial coverage was achieved by the same surgeon who completed a full coverage.

Restoration was not perfectly correlated with clinical outcomes at this time but some interesting connections may be drawn. But given that restoration has not been observed before with any other approach to treating AMD, there are no precedents to help predict the kinetics of functional recovery if it is to occur.

### Example 3: Key Regulatory Endpoints for Dry and Wet Forms of Aee-Related Macular Degeneration (AMD)

Expected efficacy endpoints are as follows: Primary Efficacy Endpoint. Change from baseline to month 12 in total area of GA lesion(s) in the study eye (in mm²) based on FAF.

Key Secondary Efficacy Endpoints. 1) Change from baseline in monocular reading speed (study eye), as assessed by Minnesota Reading (MNRead) or Radner Reading Charts at month 24 (in select countries). 2) Change from baseline in Functional Reading Independence Index (FRII) composite score, at month 24. 3) Change from baseline in normal luminance best-corrected visual acuity score (NL-BCVA) at month 24 as assessed by ETDRS chart. 4) Change from baseline in low luminance best corrected visual acuity score (LL-BCVA) at month 12 and month 24 as assessed by ETDRS chart. 5) Change from baseline in low luminance deficit (LLD) at month 12 and month 24. 6) Change from baseline at each planned assessment in the total area of GA lesion(s) in the study eye (in mm²) as assessed by FAF (in select sites). 7) Change from baseline in monocular critical print size (study eye), as assessed by MNRead or Radner Reading Charts, at month 12 and month 24 (in select countries). 8) Change from baseline in the National Eye Institute Visual Functioning Questionnaire 25 Item Version (NEI VFQ-25) distance activity subscale score at month 12 & 24. 9) Number of scotomatous points assessed by mesopic microperimetry for the evaluation of the macular functional response (Oaks study only). 10) Change in macular sensitivity as assessed by mesopic microperimetry for the evaluation of the macular functional response. 11) Systemic plasma concentration of APL-2 over time.

Safety Endpoints. 1) Incidence and severity of ocular and systemic treatment-emergent adverse events. 2) Incidence of antitherapeutic antibodies directed against APL-2. 3) Incidence of new active CNV in the study eye.

Details on some of the key secondary endpoints in dry AMD studies were as follows. Change from baseline in number of absolute scotomatous points as assessed by mesopic micrometry at week 48 [time frame: baseline, week 48]. Scotomatous points were the testing points on microperimetry examination that were centered on the macula and reported a lack of retinal sensitivity within the range tested, a maximum of 68 points were tested within this range. Higher results indicate expansion of absolute scotoma and higher number of absolute scotomatous points. Mesopic microperimetry assessments were performed post-dilation on the study eye only, and the data were forwarded to the central reading center. The data were collected up to week 48 instead of week 96, due to early termination of the study. A positive change from baseline indicates an increase in the number of absolute scotomatous points (more lack of retinal sensitivity); disease worsening.

Change from baseline in mean macular sensitivity as assessed by mesopic microperimetry at week 48 [time frame: baseline, week 48]. Mesopic microperimetry was used to assess macular sensitivity and assessments were performed post-dilation on the study eye only, and the data were forwarded to the central reading center. A negative change from baseline indicates a decrease in the mean macular sensitivity; disease worsening. The data were collected up to week 48 instead of week 96, due to early termination of the study.

Change from baseline in best corrected visual acuity (BCVA) score as assessed by early treatment diabetic retinopathy study (ETDRS) chart at week 48 [time frame: baseline, week 48]. BCVA score was based on the number of letters read correctly on the ETDRS visual acuity chart assessed at a starting distance of 4 meters (m). BCVA score testing was performed prior to dilating the eyes. BCVA score ranges from 0 to 100 letters in the study eye. The lower the number of letters read correctly on the eye chart, the worse the vision (or visual acuity). A negative change from baseline indicates a decrease in the visual acuity; disease worsening. The data were collected up to week 48 instead of week 96, due to early termination of the study.

Percentage of participants with less than 15 letters loss from baseline in BCVA score at week 48 [time frame: week 48]. Loss of less than 15 letters from baseline was assessed by the ETDRS chart at a starting distance of 4 meters (m). BCVA was measured using an eye chart and was reported as the number of letters read correctly (ranging from 0 to 100 letters). The lower the number of letters read correctly on the eye chart, the worse the vision (or visual acuity). The data were collected up to week 48 instead of week 96, due to early termination of the study.

Change from baseline in low luminance visual acuity (LLVA) as assessed by ETDRS chart under low luminance conditions at week 48 [time frame: baseline, week 48]. The LLVA was measured by placing a 2.0-log-unit neutral density filter over the best correction for that eye and having the participant read the normally illuminated ETDRS chart. The assessment was performed prior to dilating the eyes. LLVA score ranges from 0 to 100 letters in the study eye. The lower the number of letters read correctly on the eye chart, the worse the vision (or visual acuity). The data were collected up to week 48 instead of week 96, due to early termination of the study.

Percentage of participants with less than 15 letters loss from baseline in LLVA score at week 48 [time frame: week 48]. Loss of less than 15 letters from baseline was assessed by the ETDRS chart at a starting distance of 4 m. The data were collected up to week 48 instead of week 96, due to early termination of the study.

Change from baseline in binocular reading speed as assessed by Minnesota low-vision reading test (MNRead) charts or Radner reading charts at week 48 [time frame: baseline, week 48]. MNRead acuity cards were continuous-text reading-acuity cards suitable for measuring the reading acuity and reading speed of normal and low-vision participants. The MNRead acuity cards consisted of single, simple sentences with equal numbers of characters. A stopwatch was used to record time to a tenth of a second. Sentences that could not be read or were not attempted due to vision should be recorded as 0 for time and 10 for errors. The Radner Reading Cards were suitable for measuring reading speed, reading visual acuity, and critical print size. The reading test was stopped when the reading time was longer than 20 seconds or when the participant was making severe errors. A negative change from baseline indicates a decrease in the binocular reading speed; disease worsening. The data were collected up to week 48 instead of week 96, due to early termination of the study.

Change from baseline in monocular maximum reading speed as assessed by MNRead charts or Radner reading charts at week 48 [time frame: baseline, week 48]. MNRead acuity cards were continuous-text reading-acuity cards suitable for measuring the reading acuity and reading speed of normal and low-vision participants. The MNRead acuity cards consisted of single, simple sentences with equal numbers of characters. A stopwatch was used to record time to a tenth of a second. Sentences that could not be read or were not attempted due to vision should be recorded as 0 for time and 10 for errors. The Radner Reading Cards were suitable for measuring reading speed, reading visual acuity, and critical print size. The reading test was stopped when the reading time was longer than 20 seconds or when the participant was making severe errors. A negative change from baseline indicates a decrease in the monocular reading speed; disease worsening. The data were collected up to week 48 instead of week 96, due to early termination of the study.

Change from baseline in national eye institute visual functioning questionnaire 25-item (NEI VFQ-25) version composite score at week 48 [time frame: baseline, week 48]. NEI-VFQ-25 questionnaire included 25 items based on which overall composite VFQ score and 12 subscales were derived: near activities, distance activities, general health, general vision, ocular pain, vision-specific social functioning, vision-specific mental health, vision-specific role difficulties, vision-specific dependency, driving, color vision and peripheral vision. Response to each question converted to 0-100 score. Each subscale, total score=average of items contributing to score. For each subscale and total score, score range: 0 to 100, a higher score represents better functioning. A negative change from baseline indicates a decrease in the visual functioning; disease worsening. The data were collected up to week 48 instead of week 96, due to early termination of the study.

Change from baseline in NEI VFQ-25 near activity subscale score at week 48 [time frame: baseline, week]. NEI-VFQ-25 questionnaire included 25 items based on which near activities were measured. Near activities are defined as reading ordinary print in newspapers, performing work or hobbies requiring near vision, or finding something on a crowded shelf. Response to each question converted to 0-100 score. Subscale=average of items contributing to score. For this subscale the score range is 0 to 100, a higher score represents better functioning. A negative change from baseline indicates a decrease in the near visual activities; disease worsening. The data were collected up to week 48 instead of week 96, due to early termination of the study.

Change from baseline in NEI VFQ-25 distance activity subscale score at week 48 [time frame: baseline, week 48]. NEI-VFQ-25 questionnaire included 25 items based on which distance activities were measured. Distance activities are defined as reading street signs or names on stores, and going down stairs, steps, or curbs. Response to each question converted to 0-100 score. Subscale=average of items contributing to score. For this subscale the score range is 0 to 100, a higher score represents better functioning. A negative change from baseline indicates a decrease in the distance visual activities; disease worsening. The data were collected up to week 48 instead of week 96, due to early termination of the study.

Change from baseline in mean functional reading independence (FRI) index at week 48 [time frame: baseline, week 48]. The FRI was an interviewer-administered questionnaire with 7 items on functional reading activities most relevant to GA AMD participants. It has one total index score. For each FRI Index reading activity performed in the past 7 days, participants were asked about the extent to which they required vision aids, adjustments in the activity, or help from another participant. Mean FRI Index scores range from 1 to 4, with higher scores indicating greater independence. A negative change from baseline indicates a decrease in the FRI; disease worsening. The data were collected up to week 48 instead of week 96, due to early termination of the study.

### Example 4: SD-OCT Imaging for Measurement of Thickness and Area

Thickness, area and volume of different layers of retina were determined in treated eyes. SD-OCT images was captured using Spectralis (Spectralis; Heidelberg Engineering, Inc., Heidelberg, Germany), macular volume consisting of 512×49 equally spaced B-scans within a 20×20 degree field centered on the fovea. Retinal layers in all B-scans were manually segmented for thickness and area measurements using 3D-OCTOR (developed at Doheny Eye Institute). Specifically, the Outer Nuclear Layer, Photoreceptors Inner Segments (Myoid zone), Photoreceptors Outer Segments (Ellipsoid zone status), and RPE+Drusen Complex were manually segmented using all B-scans in the macular volume.

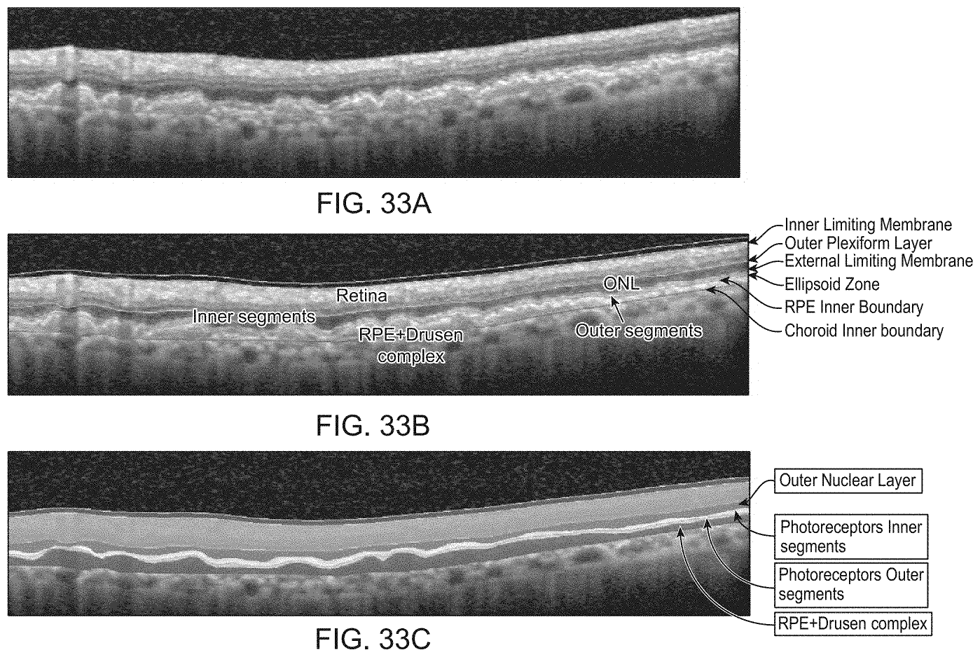

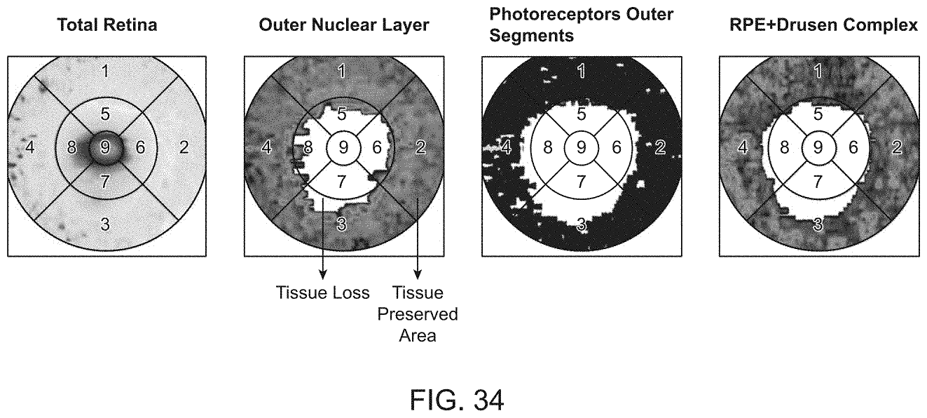

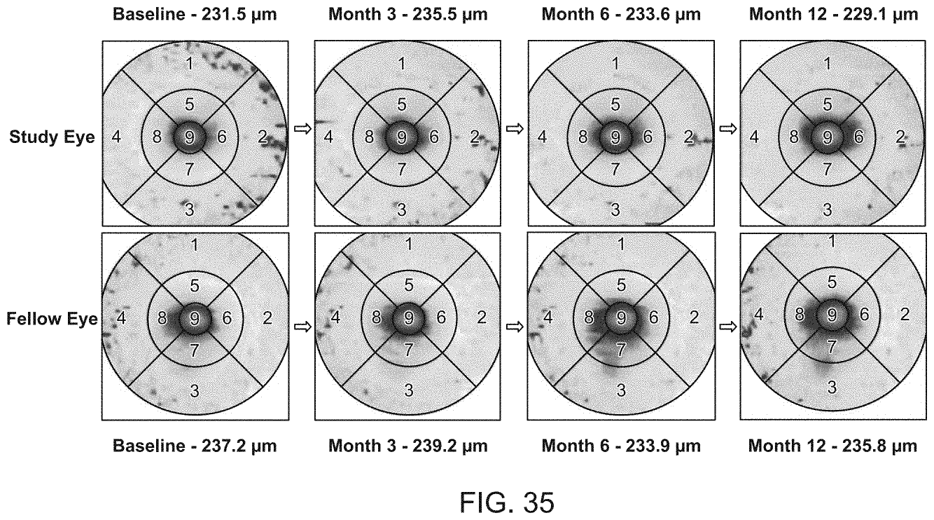

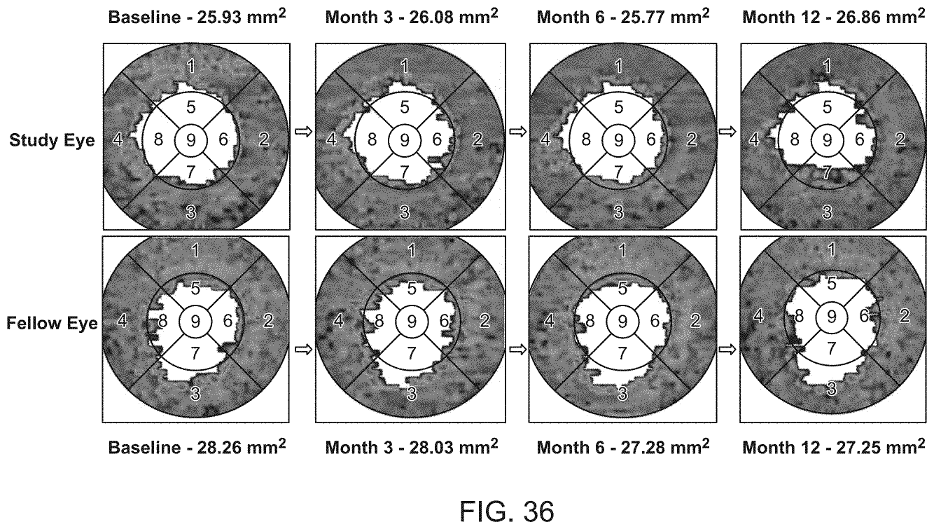

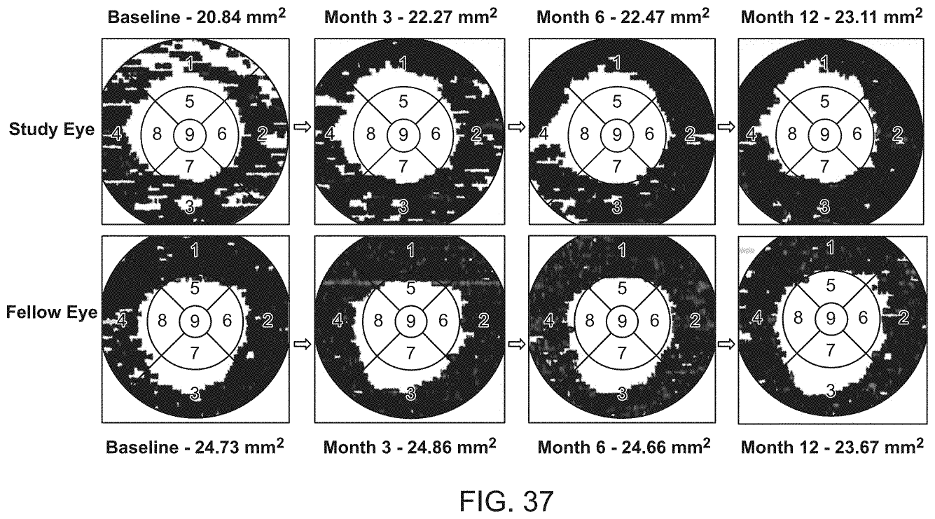

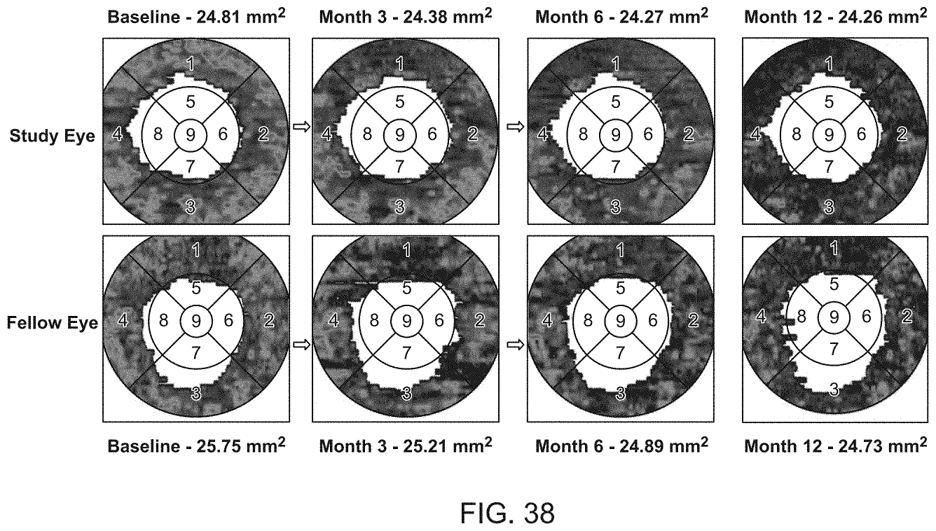

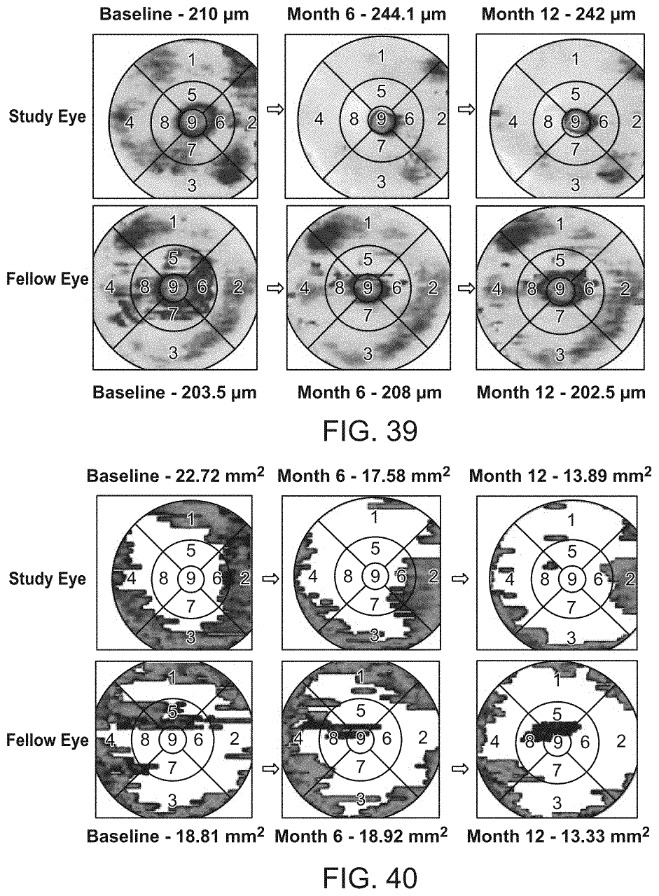

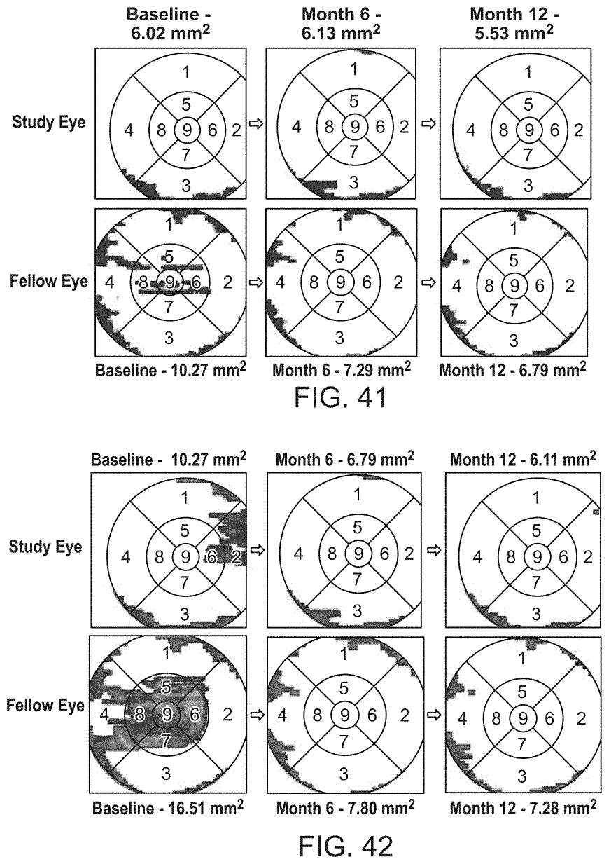

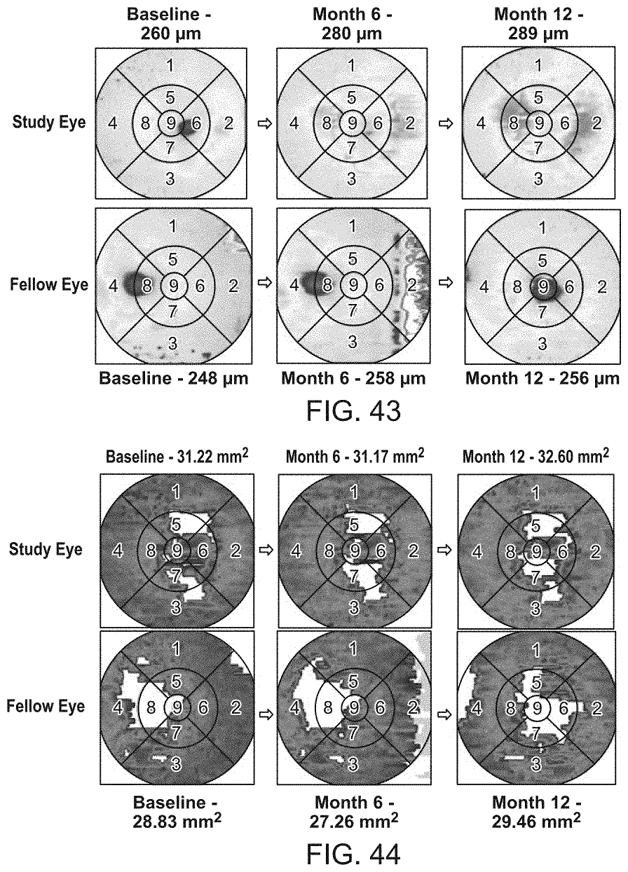

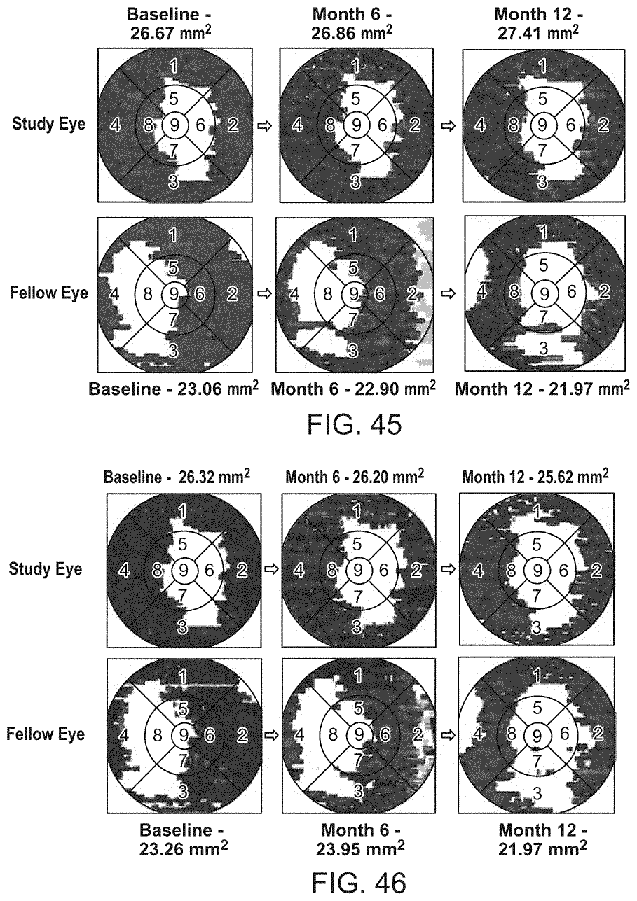

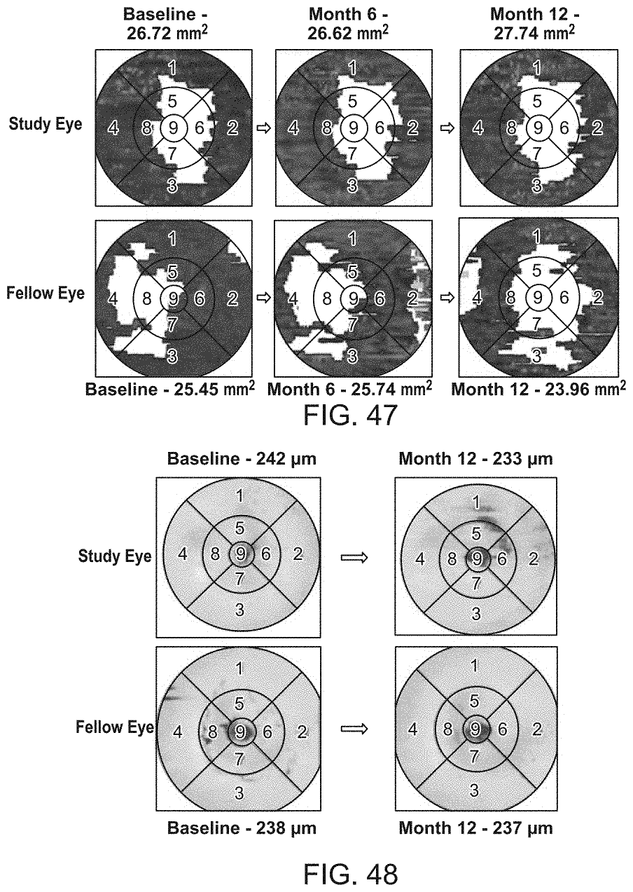

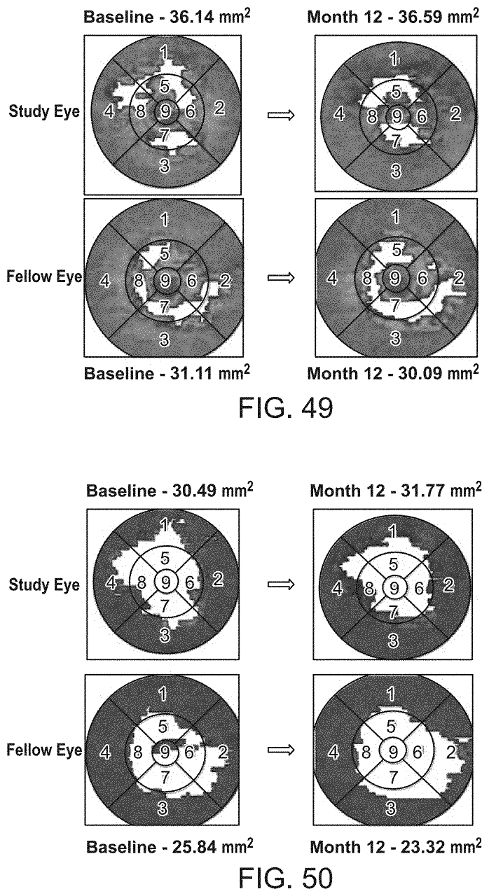

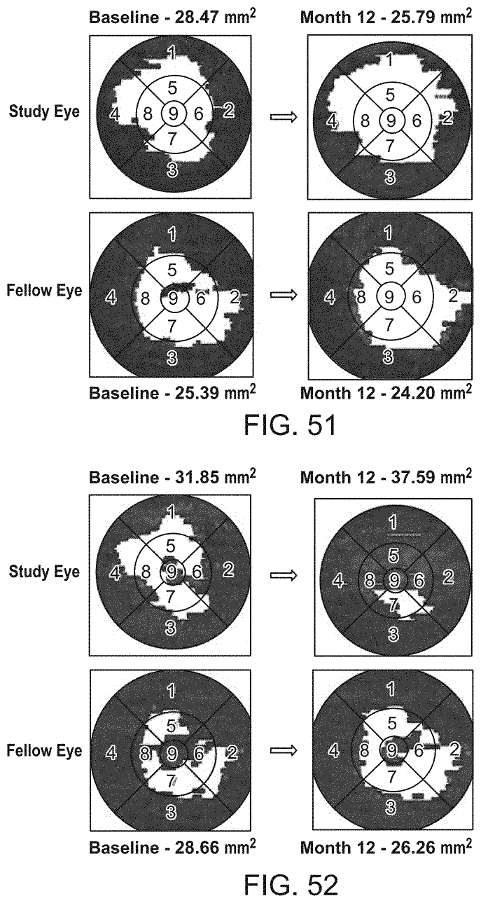

Example B scans are shown in FIGS. 33A-33C. A B-scan (FIG. 33A) was divided into layers based on boundaries (FIG. 33B), and the layer thickness and area determined (FIG. 33C). Thickness maps show thickness of the total retina, ONL, Photoreceptors Outer Segments, RPE+Drusen Complex (FIG. 34, left to right, respectively), and Photoreceptors Inner Segments. Example thickness maps for individual subjects are shown in FIGS. 35-52. Results are shown in Tables 5 to 10.

TABLE 5
SD-OCT parameters in total macular volume at baseline and Month 6 in Study eye

| Study eye at Month 6 | Baseline (N = 13 eye) | Month 6 (N = 13 eye) | p |
|---|---|---|---|
| Foveal_center_retinal_thickness_Study_Eye | 137.77 ± 74.36 | 171.72 ± 101.84 | 0.04 |
| Sub_foveal_choroidal_thickness_Study_Eye | 130.24 ± 34.79 | 131.39 ± 35.26 | 0.79 |
| ONL_Area_Study_Eye | 28.66 ± 3.96 | 28.44 ± 5.89 | 0.39 |
| ONL_Volume_Study_Eye | 1.4 ± 0.5 | 1.52 ± 0.61 | 0.33 |
| ONL_Thickness_Study_Eye | 40.4 ± 13.89 | 43.04 ± 15.72 | 0.61 |
| IS_Area_Study_Eye | 21.9 ± 7.6 | 21.24 ± 8.71 | 0.56 |
| IS_Thickness_Study_Eye | 14.31 ± 5.99 | 14.7 ± 6.54 | 0.76 |
| IS_Volume_Study_Eye | 0.5 ± 0.21 | 0.53 ± 0.25 | 0.49 |
| EZ_Area_Study_Eye | 19.01 ± 9.49 | 18.46 ± 9.97 | 0.28 |
| EZ_Thickness_Study_Eye | 8.51 ± 4.87 | 8.76 ± 5.6 | 0.59 |
| EZ_Volume_Study_Eye | 0.3 ± 0.19 | 0.32 ± 0.21 | 0.6 |
| RPE + Drusen_Complex_Area_Study_Eye | 25.01 ± 5.81 | 23.97 ± 6.89 | 0.08 |
| RPE + Drusen_Complex_Thickness_Study_Eye | 26.32 ± 9.75 | 23.27 ± 8.25 | 0.07 |
| RPE + Drusen_Complex_Volume_Study_Eye | 0.91 ± 0.35 | 0.82 ± 0.31 | 0.12 |

TABLE 6
SD-OCT parameters in total macular volume at baseline and Month 6 in Fellow eye

| Fellow eye at Month 6 | Baseline (N = 13 eyes) | Month 6 (N = 13 eyes) | p |
|---|---|---|---|
| Foveal_center_retinal_thickness_Fellow_Eye | 150.05 ± 79.36 | 140.65 ± 82.26 | 0.61 |
| Sub_foveal_choroidal_thickness_Fellow_Eye | 119.5 ± 51.21 | 117.58 ± 40.2 | 0.81 |
| ONL_Area_Fellow_Eye | 30.59 ± 4.18 | 30.53 ± 4.45 | 0.83 |
| ONL_Volume_Fellow_Eye | 1.52 ± 0.41 | 1.53 ± 0.39 | 1 |
| ONL_Thickness_Fellow_Eye | 44.31 ± 11.69 | 43.83 ± 11.11 | 0.69 |
| IS_Area_Fellow_Eye | 25.06 ± 6.6 | 23.83 ± 7.22 | 0.02 |
| IS_Thickness_Fellow_Eye | 17.66 ± 6.64 | 17.58 ± 7.23 | 0.58 |
| IS_Volume_Fellow_Eye | 0.61 ± 0.23 | 0.61 ± 0.25 | 0.67 |
| EZ_Area_Fellow_Eye | 21.24 ± 8.38 | 20.13 ± 9.18 | 0.04 |
| EZ_Thickness_Fellow_Eye | 8.84 ± 4.53 | 8.93 ± 5.2 | 0.92 |
| EZ_Volume_Fellow_Eye | 0.31 ± 0.17 | 0.31 ± 0.19 | 0.93 |
| RPE + Drusen_Complex_Area_Fellow_Eye | 27.6 ± 4.67 | 26.56 ± 6.57 | 0.16 |
| RPE + Drusen_Complex_Thickness_Fellow_Eye | 28.78 ± 6.83 | 25.9 ± 7.56 | 0.05 |
| RPE + Drusen_Complex_Volume_Fellow_Eye | 1 ± 0.24 | 0.98 ± 0.45 | 0.34 |

TABLE 7
SD-OCT parameters in total macular volume at baseline and Month 12 in Study eye

| Study eye at Month 12 | Baseline (N = 13 eye) | Month 12 (N = 13 eye) | p |
|---|---|---|---|
| Foveal_center_retinal_thickness_Study_Eye | 136.99 ± 74.36 | 189.31 ± 101.84 | 0.32 |
| Sub_foveal_choroidal_thickness_Study_Eye | 132.08 ± 34.79 | 140.62 ± 35.26 | 0.14 |
| ONL_Area_Study_Eye | 29.14 ± 3.96 | 29.25 ± 5.89 | 0.76 |
| ONL_Volume_Study_Eye | 1.48 ± 0.5 | 1.83 ± 0.61 | 0.39 |
| ONL_Thickness_Study_Eye | 42.27 ± 13.89 | 52.14 ± 15.72 | 0.56 |
| IS_Area_Study_Eye | 23.14 ± 7.6 | 21.81 ± 8.71 | 0.14 |
| IS_Thickness_Study_Eye | 15.42 ± 5.99 | 15.09 ± 6.54 | 0.71 |
| IS_Volume_Study_Eye | 0.54 ± 0.21 | 0.54 ± 0.25 | 0.81 |
| EZ_Area_Study_Eye | 21.15 ± 9.49 | 19.94 ± 9.97 | 0.04 |
| EZ_Thickness_Study_Eye | 9.38 ± 4.87 | 10.11 ± 5.6 | 0.21 |
| EZ_Volume_Study_Eye | 0.33 ± 0.19 | 0.37 ± 0.21 | 0.15 |
| RPE + Drusen_Complex_Area_Study_Eye | 25.24 ± 5.81 | 24.68 ± 6.89 | 0.24 |
| RPE + Drusen_Complex_Thickness_Study_Eye | 25 ± 9.75 | 22.51 ± 8.25 | 0.25 |
| RPE + Drusen_Complex_Volume_Study_Eye | 0.87 ± 0.35 | 0.81 ± 0.31 | 0.39 |

TABLE 8
SD-OCT parameters in total macular volume at baseline and Month 12 in Fellow eye

| Fellow eye at Month 12 | Baseline (N = 13 eye) | Month 12 (N = 13 eye} | P |
|---|---|---|---|
| Foveal_center_retinal_thickness_Fellow_Eye | 152.1 ± 84.57 | 134.24 ± 57.84 | 0.88 |
| Sub_foveal_choroidal_thickness_Fellow_Eye | 125.92 ± 47.55 | 124.84 ± 39.68 | 0.64 |
| ONL_Area_Fellow_Eye | 30.08 ± 4.28 | 29.4 ± 5.76 | 0.54 |
| ONL_Volume_Fellow_Eye | 1.5 ± 0.34 | 1.51 ± 0.45 | 0.9 |
| ONL_Thickness_Fellow_Eye | 43.58 ± 10 | 43.56 ± 12.96 | 0.54 |
| IS_Area_Fellow_Eye | 25.23 ± 6.49 | 23.49 ± 7.01 | 0.01 |
| IS_Thickness_Fellow_Eye | 17.86 ± 5.5 | 16.24 ± 5.46 | 0.09 |
| IS_Volume_Fellow_Eye | 0.62 ± 0.2 | 0.57 ± 0.2 | 0.1 |
| EZ_Area_Fellow_Eye | 22.8 ± 6.49 | 21.25 ± 7.24 | 0.01 |
| EZ_Thickness_Fellow_Eye | 10.01 ± 4.25 | 10.9 ± 5.7 | 0.51 |
| EZ_Volume_Fellow_Eye | 0.35 ± 0.16 | 0.38 ± 0.2 | 0.37 |
| RPE + Drusen_Complex_Area_Fellow_Eye | 26.89 ± 4.64 | 25.12 ± 6.79 | 0.04 |
| RPE + Drusen_Complex_Thickness_Fellow_Eye | 26.38 ± 6 | 22.19 ± 6.31 | 0.02 |
| RPE + Drusen_Complex_Volume_Fellow_Eye | 0.92 ± 0.24 | 0.8 ± 0.26 | 0.05 |

TABLE 9
SD-OCT parameters in total macular volume at baseline and Month 6 in Study eye—Cohort 4

| Study eye at Month 6 | Baseline (N = 3 eye) | Month 6 (N = 3 eye) | p |
|---|---|---|---|
| Foveal_center_retinal_thickness_Study_Eye | 201.1 ± 51.15 | 255.3 ± 109.92 | 0.29 |
| Sub_foveal_choroidal_thickness_Study_Eye | 128 ± 22.52 | 126.34 ± 10.07 | 0.6 |
| ONL_Area_Study_Eye | 32.29 ± 1.22 | 33.76 ± 2.29 | 0.29 |
| ONL_Volume_Study_Eye | 2.03 ± 0.11 | 2.04 ± 0.17 | 1 |
| ONL_Thickness_Study_Eye | 58.87 ± 1.26 | 55.94 ± 6.45 | 0.6 |
| IS_Area_Study_Eye | 27.51 ± 0.74 | 28.87 ± 2.1 | 0.11 |
| IS_Thickness_Study_Eye | 19.04 ± 4.58 | 21.1 ± 2.27 | 0.6 |
| IS_Volume_Study_Eye | 0.65 ± 0.13 | 0.77 ± 0.14 | 0.29 |
| EZ_Area_Study_Eye | 26.54 ± 2.08 | 27.11 ± 2.4 | 0.29 |
| EZ_Thickness_Study_Eye | 12.84 ± 2.42 | 13.84 ± 5.81 | 0.6 |
| EZ_Volume_Study_Eye | 0.44 ± 0.09 | 0.5 ± 0.22 | 0.6 |
| RPE + Drusen_Complex_Area_Study_Eye | 28.3 ± 1.48 | 28.63 ± 2.14 | 1 |
| RPE + Drusen_Complex_Thickness_Study_Eye | 28.17 ± 5.23 | 25.94 ± 2.44 | 0.6 |
| RPE + Drusen_Complex_Volume_Study_Eye | 0.98 ± 0.21 | 0.93 ± 0.07 | 0.6 |

TABLE 10
SD-OCT parameters in total macular volume at baseline and Month 6 in Fellow eye—Cohort 4

| Fellow eye at Month 6 | Baseline (N = 4 eyes) | Month 6 (N = 4 eyes) | p |
|---|---|---|---|
| Foveal_center_retinal_thickness_Fellow_Eye | 179.9 ± 82.47 | 166.08 ± 104.85 | 0.49 |
| Sub_foveal_choroidal_thickness_Fellow_Eye | 114.25 ± 33.88 | 110.5 ± 24.29 | 0.69 |
| ONL_Area_Fellow_Eye | 32.04 ± 2.6 | 31.95 ± 4.09 | 0.93 |
| ONL_Volume_Fellow_Eye | 1.92 ± 0.18 | 1.86 ± 0.2 | 0.45 |
| ONL_Thickness_Fellow_Eye | 56.88 ± 3.42 | 54.4 ± 6.44 | 0.44 |
| IS_Area_Fellow_Eye | 27.87 ± 3.4 | 27.66 ± 3.78 | 0.82 |
| IS_Thickness_Fellow_Eye | 23.35 ± 6.28 | 24.23 ± 4.09 | 0.65 |
| IS_Volume_Fellow_Eye | 0.79 ± 0.2 | 0.83 ± 0.11 | 0.59 |
| EZ_Area_Fellow_Eye | 25.34 ± 3.03 | 24.27 ± 3.76 | 0.24 |
| EZ_Thickness_Fellow_Eye | 10.83 ± 3.77 | 10.83 ± 5.29 | 1 |
| EZ_Volume_Fellow_Eye | 0.37 ± 0.14 | 0.37 ± 0.17 | 0.96 |
| RPE + Drusen_Complex_Area_Fellow_Eye | 29.67 ± 3.38 | 27.95 ± 4.16 | 0.05 |
| RPE + Drusen_Complex_Thickness_Fellow_Eye | 31.13 ± 7.39 | 28.55 ± 8.04 | 0.1 |
| RPE + Drusen_Complex_Volume_Fellow_Eye | 1.05 ± 0.26 | 1.26 ± 0.72 | 0.49 |

TABLE 11
SD-OCT parameters in total macular volume at baseline and Month 12 in Study eye—Cohort 4

| Study eye at Month 12 | Baseline (N = 4 eye) | Month 12 (N = 4 eye) | p |
|---|---|---|---|
| Foveal_center_retinal_thickness_Study_Eye | 188.15 ± 49.14 | 224.73 ± 107.71 | 0.47 |
| Sub_foveal_choroidal_thickness_Study_Eye | 134.5 ± 22.52 | 142 ± 41.28 | 0.72 |
| ONL_Area_Study_Eye | 33.25 ± 2.17 | 34.44 ± 1.39 | 0.15 |
| ONL_Volume_Study_Eye | 2 ± 0.11 | 2.08 ± 0.3 | 1 |
| ONL_Thickness_Study_Eye | 56.4 ± 5.04 | 56.13 ± 10.57 | 0.72 |
| IS_Area_Study_Eye | 27.75 ± 0.78 | 27.78 ± 1.98 | 0.72 |
| IS_Thickness_Study_Eye | 19.25 ± 3.77 | 17.6 ± 2.42 | 0.47 |
| IS_Volume_Study_Eye | 0.69 ± 0.13 | 0.66 ± 0.1 | 0.47 |
| EZ_Area_Study_Eye | 26.88 ± 1.84 | 26.33 ± 2.36 | 0.47 |
| EZ_Thickness_Study_Eye | 12.45 ± 2.12 | 14.88 ± 2.83 | 0.07 |
| EZ_Volume_Study_Eye | 0.44 ± 0.08 | 0.55 ± 0.1 | 0.07 |
| RPE + Drusen_Complex_Area_Study_Eye | 29.19 ± 2.15 | 29.45 ± 1.8 | 0.47 |
| RPE + Drusen_Complex_Thickness_Study_Eye | 26.45 ± 5.48 | 24.15 ± 2.97 | 0.28 |
| RPE + Drusen_Complex_Volume_Study_Eye | 0.94 ± 0.19 | 0.9 ± 0.09 | 0.47 |

TABLE 12
SD-OCT parameters in total macular volume at baseline and Month 12 in Fellow eye—Cohort 4

| Fellow eye at Month 12 | Baseline (N = 4 eye) | Month 12 (N = 4 eye) | p |
|---|---|---|---|
| Foveal_center_retinal_thickness_Fellow_Eye | 197.5 ± 83.87 | 146.5 ± 28.24 | 0.5 |
| Sub_foveal_choroidal_thickness_Fellow_Eye | 123.67 ± 44.53 | 112.34 ± 48.51 | 0.28 |
| ONL_Area_Fellow_Eye | 30.77 ± 1.8 | 30.5 ± 1.29 | 0.63 |
| ONL_Volume_Fellow_Eye | 1.77 ± 0.12 | 1.81 ± 0.25 | 0.69 |
| ONL_Thickness_Fellow_Eye | 52.97 ± 2.46 | 54.3 ± 7.16 | 0.69 |
| IS_Area_Fellow_Eye | 26.6 ± 3.98 | 25.16 ± 3.29 | 0.22 |
| IS_Thickness_Fellow_Eye | 22.94 ± 2.41 | 18.24 ± 2.98 | 0.11 |
| IS_Volume_Fellow_Eye | 0.77 ± 0.1 | 0.61 ± 0.1 | 0.11 |
| EZ_Area_Fellow_Eye | 26.13 ± 3.3 | 24.85 ± 3.26 | 0.01 |
| EZ_Thickness_Fellow_Eye | 13.3 ± 3.35 | 17.47 ± 3.99 | 0.15 |
| EZ_Volume_Fellow_Eye | 0.45 ± 0.13 | 0.58 ± 0.14 | 0.15 |
| RPE + Drusen_Complex_Area_Fellow_Eye | 28.33 ± 2.73 | 25.35 ± 2.43 | 0.02 |
| RPE + Drusen_Complex_Thickness_Fellow_Eye | 23.77 ± 3.43 | 22.84 ± 5.35 | 0.55 |
| RPE + Drusen_Complex_Volume_Fellow_Eye | 0.8 ± 0.14 | 0.76 ± 0.18 | 0.3 |

### Example 5

RPE Treatment Leads to Blood-Retinal Barrier Restoration.

RPE secretes very high levels of PEDF (For OpRegen levels of 2000-4000 ng/ml/day were measured), which contribute to its therapeutic potency. PEDF is a 50 kDa protein secreted by RPE and Muller glia in vivo with beneficial functions such as anti-angiogenic activities, neuroprotective functions for photoreceptors, possibly through restoring mitochondrial dynamics perturbed by aging and oxidative stress, anti-inflammatory activity (through its interaction with master factor NF-KappaB), and anti-fibrotic activity via binding to extracellular matrix (collagen and proteoglycans). In OpRegen-treated subjects, this is evidenced by the fluorescein angiography (FA) improvement in those with/without drusen, and OCT imaging with possible signs of ECM remodeling or scar attenuation within the GA lesion seen as early as 2-4 weeks post-transplant.

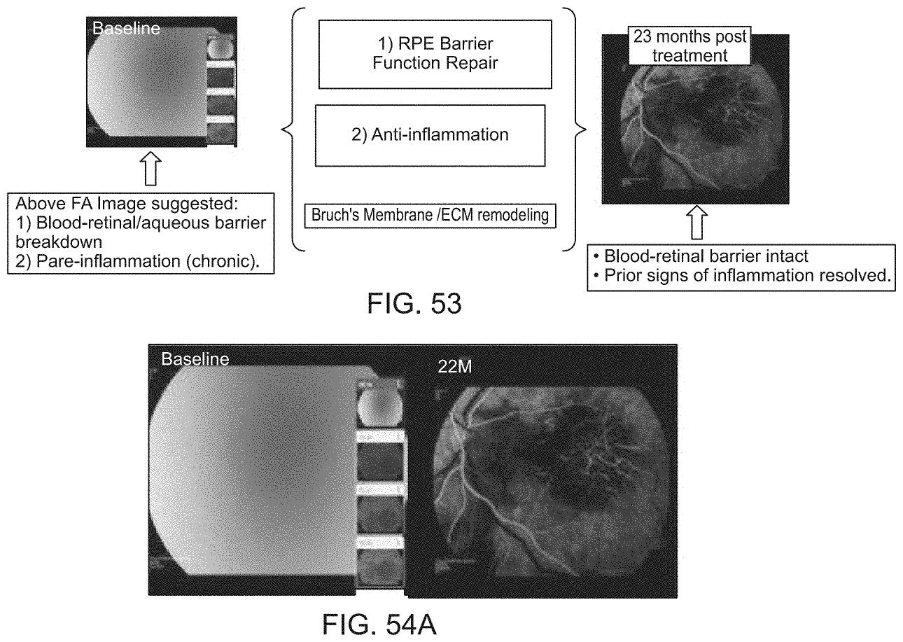

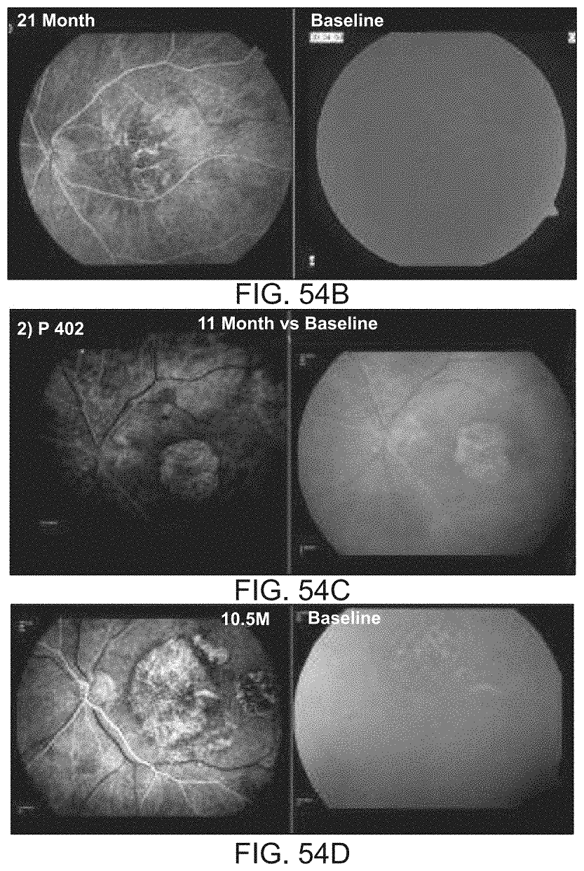

A baseline FA exam in subject 8 showed massive fluorescein dye leaking into the vitreous cavity, which blocks the visibility of vascular perfusion during the choroidal flush and arterial phase, suggesting blood-retinal barrier breakdown and para-inflammation pre-existing within the eye (FIG. 53). At 22 months post-transplant, an FA exam showed clear choroidal and retinal vascular perfusion, and there was no dye leaking into the vitreous cavity, indicating that OpRegen restored the integrity of the broken BRB, possibly through multiple mechanisms of action such as via PEDF various subjects. FIGS. 54A-54D provide three additional case examples of BRB restoration or repair by OpRegen cell therapy.

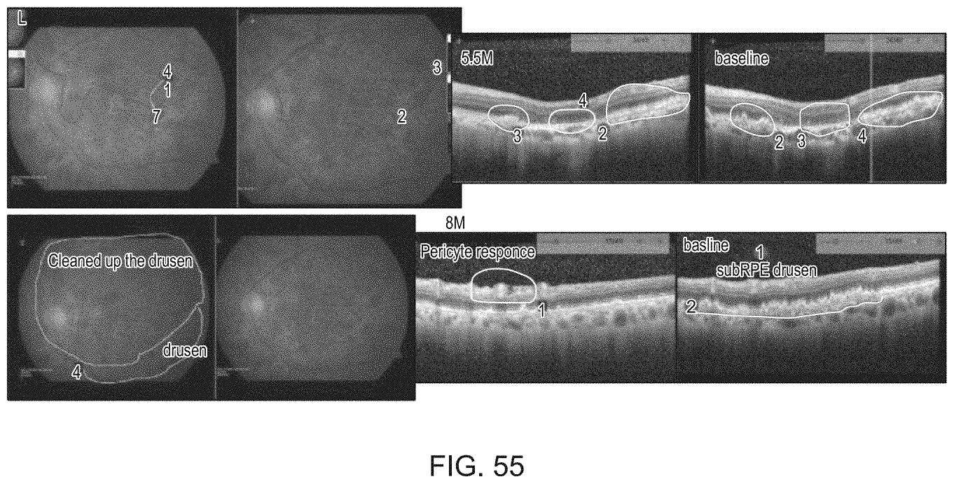

Subject 8 is a typical example of a patient with extensive drusen spreading over the entire posterior retina. FIG. 55 shows that drusen resolution started from the graft area at the superior (top left), then moved down cleaning up almost the entire posterior, except a small elongated band that remained at 8 months post-op (top, second from left, large circle). OCT imaging features are in concert with color fundus photography, at 5.5 months (top, second from right) and 8 months (bottom, second from right), compared to baseline (top right and bottom right); subRPE drusen was significant reduced or resolved. The host retinal texture seems attenuating, which suggests possible ECM remodeling, partially and possibly due to the biological effects rendered by high level PEDF presence.

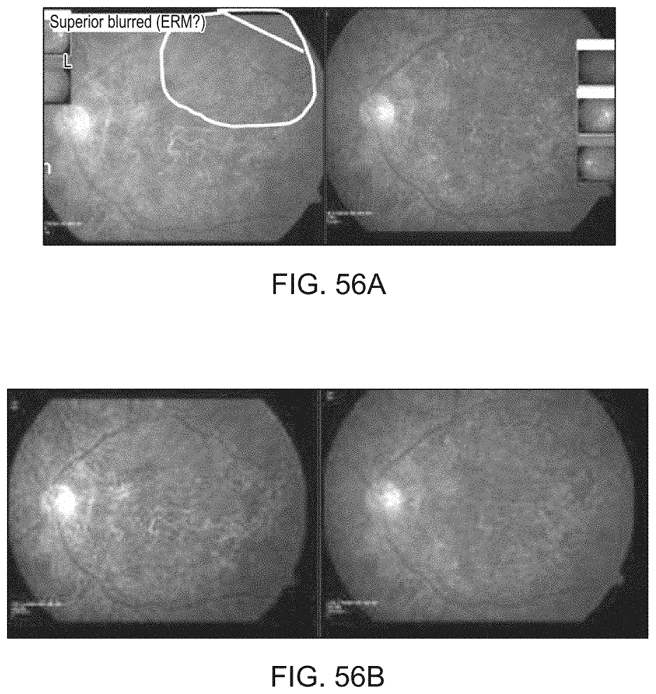

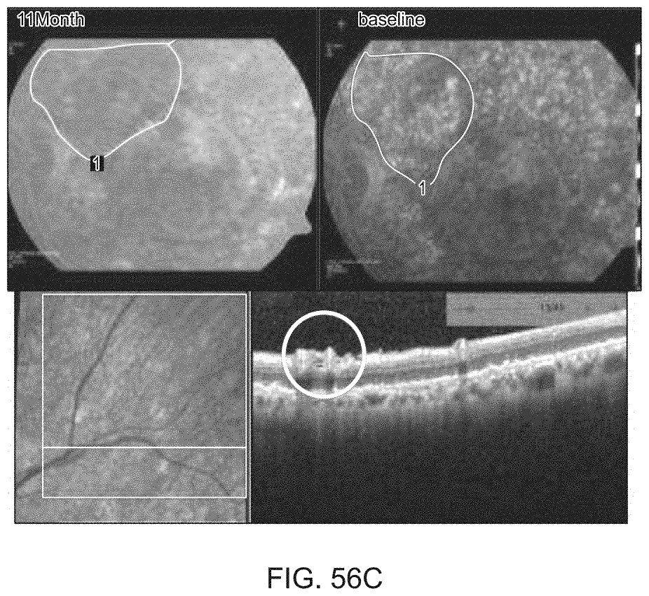

At 11 months, in subject 8, grafts continued to remodel the host retina after the large drusen resolved (FIGS. 56A-56C). FA showed significantly reduced staining (drusen), yet, appeared to have a membrane-like veil that blurs the retina vascular architecture. At 22 months on the color fundus exam, the retinal tissue appears sharper compared to that on baseline, possibly because of its anti-inflammatory effects, or ECM cleanings for which PEDF has a role in regulating the extra-matrix turnover.

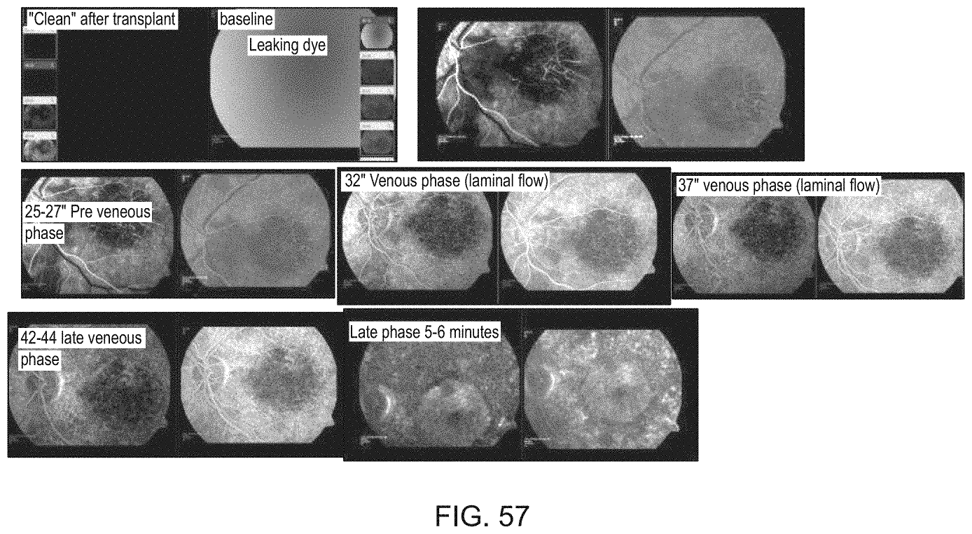

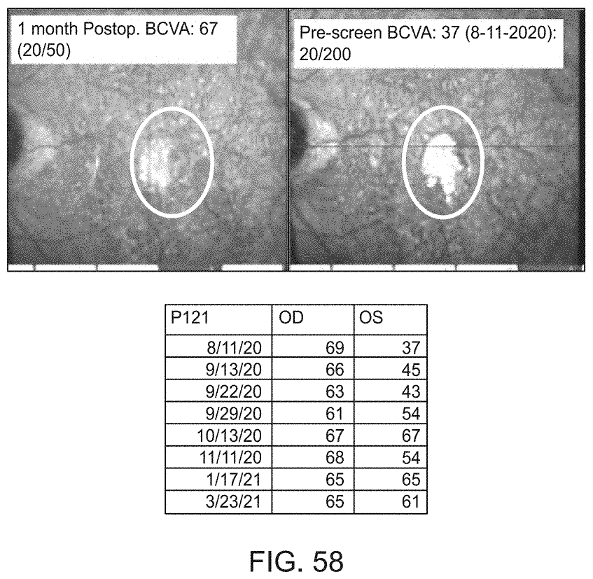

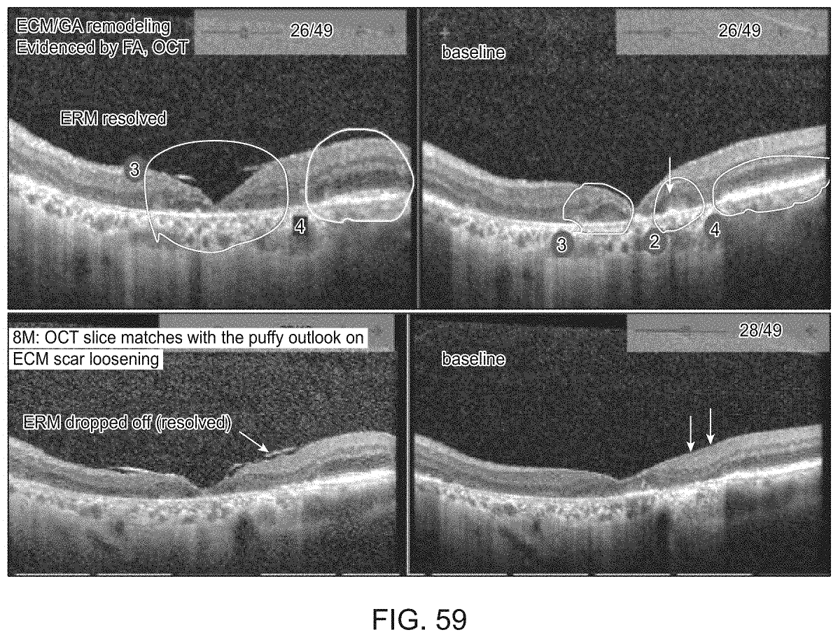

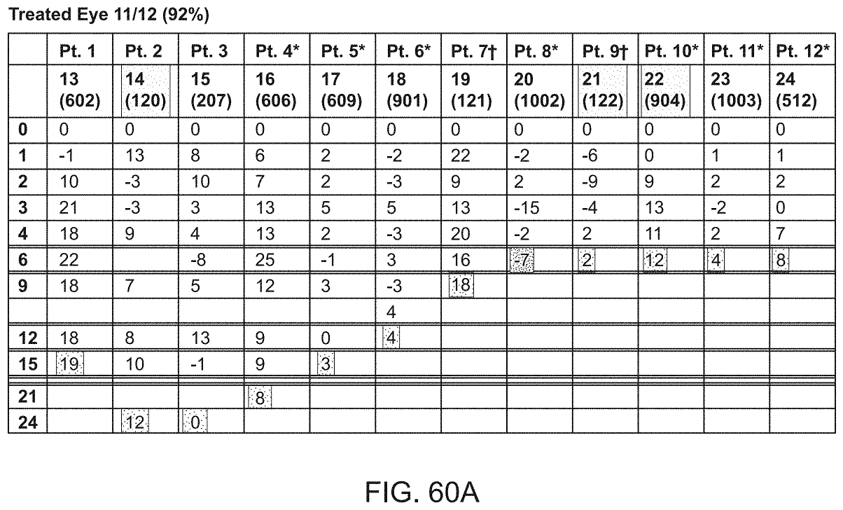

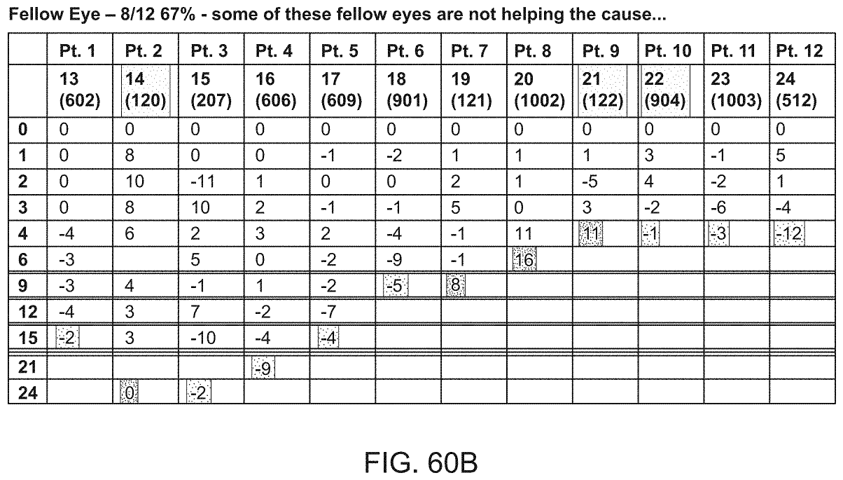

FIG. 57 provides the time course FA exams from early phase, mid-phase and late phase, demonstrating a significant improvement of retinal health with better visibility of vascular perfusion throughout, and reduced inflammation. Retinal tissue appears very clean; this FA pattern has not been reported before in other therapeutic modalities. This is very unique to the therapeutic effects of OpRegen.

All references (including all non-patent literature, patents, and patent publications) provided herein are incorporated herein by reference in their entireties.

1. A method of treating or slowing the progression of a retinal disease or disorder comprising administering a cell therapeutic agent to a subject in need thereof, wherein the cell therapeutic agent comprises retinal pigment epithelium (RPE) cells, and wherein the RPE cells restore the anatomy or functionality of a retina of the subject.

2. The method of claim 1, wherein the RPE cells are derived from pluripotent cells.

3. The method of claim 1 where the RPE cells are human RPE cells.

4. The method of claim 3, wherein the RPE cells were derived from a human embryonic (hESC) cell line.

5. The method of claim 4, wherein the RPE cells were derived under low oxygen (5%) culture supplemented with high concentration of Activin A, a transforming growth factor beta (TGF-b) family member, and nicotinamide before switching to normal oxygen (20%) culture to enrich RPE population.

6. The method of claim 1, wherein the RPE cells secrete PEDF at a concentration of about 2000 ng/ml/day to about 4000 ng/ml/day.

7. The method of claim 1, wherein the cell therapeutic agent is administered to a region of the atrophic retina or adjacent to a region of the atrophic retina of the patient.

8. The method of claim 1, wherein the cell therapeutic agent is administered at a dose of about 50,000 cells to about 1,000,000 cells.

9. The method of claim 1, wherein the cell therapeutic agent is administered at a dose of about 100,000 cells to about 750,000 cells.

10. The method of claim 9, wherein the cell therapeutic agent is administered at a dose of about 200,000 cells to about 500,000 cells.

11. The method of claim 1, wherein the administration of the cell therapeutic agent decreases the atrophy area in an atrophic retina of the subject.

12. The method of claim 1, wherein the administration of the cell therapeutic agent restores one or more retinal layers of the retina.

13. The method of claim 1, wherein the administration of the cell therapeutic agent restores the functionality of photoreceptors in the retina.

14. The method of claim 1, wherein the administration of the cell therapeutic agent restores the outer nuclear layer (ONL) of the retina.

15. The method of claim 1, wherein the administration of the cell therapeutic agent restores the ellipsoid zone (EZ) of the retina.

16. The method of claim 1, wherein the administration of the cell therapeutic agent restores the fovea of the retina.

17. The method of claim 1, wherein the administration of the cell therapeutic agent restores the blood-retinal barrier (BRB) of the retina.

18. The method of claim 1, wherein the administration of the cell therapeutic agent remodels the extracellular matrix (ECM) of the retina.

19. The method of claim 1, wherein the restoring of the anatomy or functionality of the retina is determined by assessing one or more of reduced growth of geographic atrophy, improvement of visual acuity, improvement of reading speed, improvement of retinal structure, reductions in drusen, or stable engraftment of cells.

20. The method of claim 19, wherein the improvement is measured by microperimetry.

21. The method of claim 1, wherein the vision of the subject is improved by treatment, and wherein the improved vision is assessed by one or more of: change in total area of GA lesion(s); change in monocular reading speed; change in Functional Reading Independence Index (FRII) composite score; change in normal luminance best-corrected visual acuity score (NL-BCVA); change in low luminance best corrected visual acuity score (LL-BCVA); change in low luminance deficit (LLD); change in monocular critical print size; change in the National Eye Institute Visual Functioning Questionnaire 25 Item Version (NEI VFQ-25) distance activity subscale score; change in number of scotomatous points; change in macular sensitivity; and change in systemic plasma concentration of APL-2.

22. The method of claim 1, wherein the method results in minimal or no delayed inflammation of rejection of implanted cells.

23. The method of claim 1, wherein administering comprises delivering the RPE cells to a region of the retina or adjacent to the retina.

24. The method of claim 23, wherein delivering comprises implanting the RPE cells in a region of the retina or adjacent to the retina.

25. The method of claim 1, wherein the treating comprises the pluripotent secretory effects of the RPE cells.

26. The method of claim 25, wherein the subject suffers from a retinal disease condition selected from Dry AMD, retinitis pigmentosae, usher syndrome, vitelliform maculopathy, Stargardt disease, retinal detachment, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, pathologic myopia, retinitis pigmentosa, and macular degeneration.

27. The method of claim 1, wherein the cell therapeutic agent is administered with a delivery device.

28. The method of claim 27, wherein the cell therapeutic agent is administered to or adjacent to a geographic atrophy of the retina with the delivery device.

29. The method of claim 27, wherein the delivery device comprises a needle, a capillary and a tip.

30. The method of claim 29, wherein the delivery device comprises a needle with an outer diameter of about 0.63 mm and an inner diameter of about 0.53 mm, a capillary with an outer diameter of about 0.5 mm and an inner diameter of about 0.25 mm, and a tip with an outer diameter of about 0.12 mm and an inner diameter of about 0.07 mm.

31-34. (canceled)
